(19) United States
(12) Patent Application Publication
(10) Pub. No.: US 2025/0136548 A1
(43) Pub. Date: May 1, 2025

(54) FUNCTIONALIZED ALKYLAMINES

(71) Applicant: Terran Biosciences Inc., Miami Beach, FL (US)

(72) Inventors: Matthew DUNCTON, Las Vegas, NV (US); Samuel CLARK, Miami, FL (US); Scott MILLER, Karlskoga (SE)

(21) Appl. No.: 18/730,513

(22) PCT Filed: Jan. 20, 2023
(86) PCT No.: PCT/US2023/061030

§ 371 Date: Jul. 19, 2024

Related U.S. Application Data
(60) Provisional application No. 63/301,341, filed on Jan. 20, 2022.

(51) Int. Cl. C07D 209/08; A61K 31/4045; A61K 31/4439; C07D 401/06; C07D 403/06

(52) U.S. Cl. CPC C07D 209/08; A61K 31/4045; A61K 31/4439; C07D 401/06; C07D 403/06

(57) ABSTRACT

Certain aspects of the present disclosure include compounds having Formula (X). Disclosed embodiments also include compositions comprising at least one compound according to the present disclosure, such as from 5% to 70% of at least one compound according to the present disclosure, at a dosage of from 0.1 mg to about 10,000 mg, and further comprising an excipient, an adjuvant, a carrier, and/or at least one additional biologically active compound other than a compound according to the present disclosure. Disclosed compounds and/or can be administered to a subject multiple times per day, such as 1 to 3 times per day, and/or periodically, such as every 2 to 7 days, for a variety of reasons, such as to increase neuronal plasticity, to treat a neurological disease, to treat a neuropsychiatric disease, to elicit a biological response, such as by activating a 5-HT_{2A} receptor, or to increase at least one of translation, transcription, or secretion of neurotrophic factors. A method for making disclosed compounds also is provided.

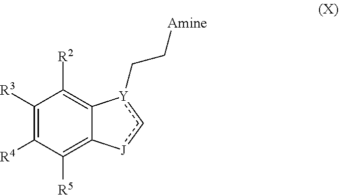

### CROSS REFERENCE TO RELATED APPLICATIONS

This application claims priority to and the benefit of U.S. Provisional Application 63/301,341, filed Jan. 20, 2022, which is incorporated by reference herein in entirety for all purposes.

### FIELD

The present application concerns functionalized alkylamines, with certain disclosed embodiments concerning amine derivatives of tryptamine, as well as embodiments of a method for using and making such derivatives.

### BACKGROUND

Bacteria in the human gut convert dietary tryptophan to tryptamine. Tryptanine is an indolamine that includes a 2-aminoethyl group at the third position of the indole ring system, as shown below.

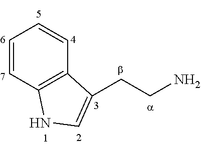 [SMILES: NCCc1c[nH]c2ccccc12]

The tryptarnine ring structure is included in a number of aminergic neuromodulators, such as melatonin and serotonin. The tryptamine ring structure is also present in a number of psychedelic compounds, including dimethyltryptamine, (DMT) and psilocybin. Tryptamine activates trace amine-associated receptors and regulates dopaminergic and serotonergic systems in mammals. Tryptamine-derived drugs have been developed to treat a number of maladies, such as migraines, and trace amine-associated receptors are being considered for treating neuropsychiatric disorders. The need exists for new and effective functionalized alkylamines, such as tryptamine derivatives.

### SUMMARY

Several aspects of the present disclosure relate to compounds having Formula (X)

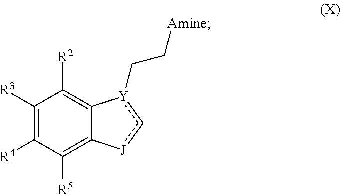

or a pharmaceutically acceptable salt or isomer thereof, the compound comprising an indole ring system, wherein
J and Y are each independently C, CH, N, or NH;
one “ [symbol] ” is a single bond and one “ [symbol] ” is a double bond;
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl;
R^{6b}, R^{6c} and R^{6d} are each independently H or C₁₋₆ alkyl; and
“Amine” represents an amine selected from:

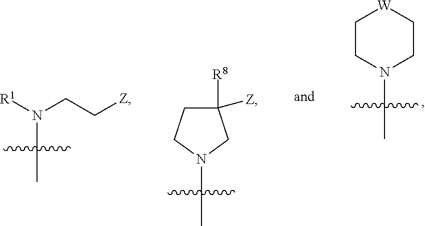

wherein
R¹ is H, C₁₋₆ alkyl, or C₄₋₁₄ alkyl-cycloalkyl;
Z is —OH, —SR^{7a}, —S(O)R^{7a}, —S(O)₂R^{7a}, —N(R^{7b}R^{7c}), or —N(R^{7b})C(O)R^{7c};
R^{7a}, R^{7b} and R^{7c} are each independently H or C₁₋₆ alkyl;
R⁸ is H or methyl;
W is —C(R^{7a})(Z); and
“ [symbol] ” indicates a point of attachment to the indole ring system, with the proviso that the compound is not

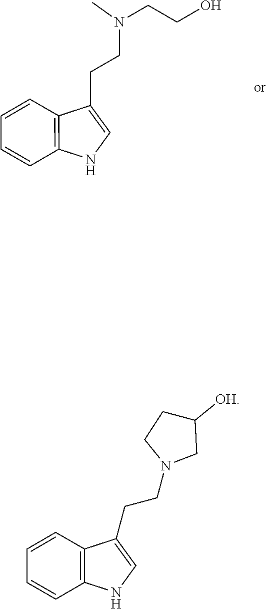 [SMILES: CN(CCO)CCc1c[nH]c2ccccc12 ; OC1CCN(CCc2c[nH]c3ccccc23)C1]

Several embodiments of the compounds of Formula (X) are compounds having a Formula (I)

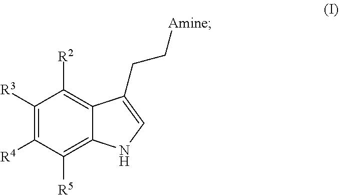

or a pharmaceutically acceptable salt or isomer thereof, the compound comprising an indole ring system, wherein
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl;
R^{6b}, R^{6c} and R^{6d} are each independently H or C₁₋₆ alkyl; and
“Amine” represents an amine selected from:

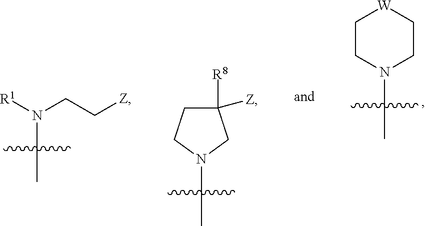

wherein
R¹ is H, C₁₋₆ alkyl, or C₄₋₁₄ alkyl-cycloalkyl;
Z is —OH, —SR^{7a}, —S(O)R^{7a}, —S(O)₂R^{7a}, —N(R^{7b}R^{7c}), or —N(R^{7b})C(O)R^{7c};
R^{7a}, R^{7b} and R^{7c} are each independently H or C₁₋₆ alkyl;
R⁸ is H or methyl;
W is —C(R^{7a})(Z); and
“ [symbol] ” indicates a point of attachment to the indole ring system.

Some embodiments of the compounds of Formula (X) are compounds having a Formula (II)

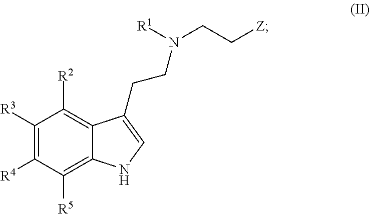

or a pharmaceutically acceptable salt or isomer thereof, wherein
R¹ is H, C₁₋₆ alkyl, or C₄₋₁₄ alkyl-cycloalkyl;
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl;
R^{6b}, R^{6c} and R^{6d} are each independently H or C₁₋₆ alkyl; and
Z is —OH, —SR^{7a}, —S(O)R^{7a}, —S(O)₂R^{7a}, —N(R^{7b}R^{7c}), or —N(R^{7b})C(O)R^{7c}; and
R^{7a}, R^{7b} and R^{7c} are each independently H or C₁₋₆ alkyl.

Some embodiments of the compounds of Formula (X) are compounds having a Formula (III)

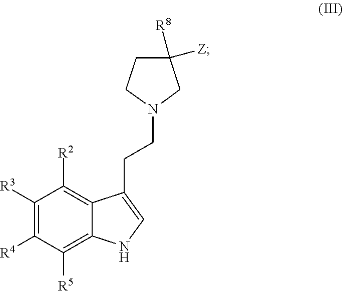

or a pharmaceutically acceptable salt or isomer thereof, wherein
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl;
R^{6b}, R^{6c} and R^{6d} are each independently H or C₁₋₆ alkyl;
R⁸ is H or methyl; and
Z is —OH, —SR^{7a}, —S(O)R^{7a}, —S(O)₂R^{7a}, —N(R^{7b}R^{7c}), or —N(R^{7b})C(O)R^{7c}; and
R^{7a}, R^{7b} and R^{7c} are each independently H or C₁₋₆ alkyl.

Some embodiments of the compounds of Formula (X) are compounds having a Formula (IV)

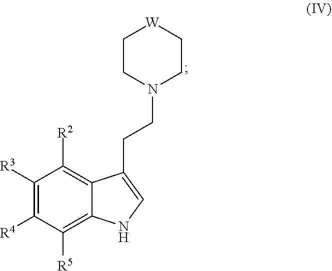

or a pharmaceutically acceptable salt or isomer thereof, wherein
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl;
R^{6b}, R^{6c} and R^{6d} are each independently H or C₁₋₆ alkyl;
W is —C(R^{7a})(Z);
Z is OH, S, —S(O), —S(O)₂, —N(R^{7a}), or —NC(O)R^{7a}; and
R^{7a} is H or C₁₋₆ alkyl.

Some embodiments of the compounds of Formula (X) are compounds having a Formula (V)

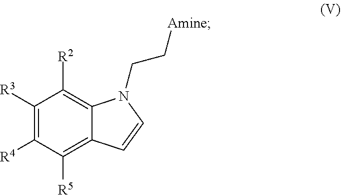

or a pharmaceutically acceptable salt or isomer thereof, the compound comprising an indole ring system, wherein
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl;
R^{6b}, R^{6c} and R^{6d} are each independently H or C₁₋₆ alkyl; and
“Amine” represents an amine selected from:

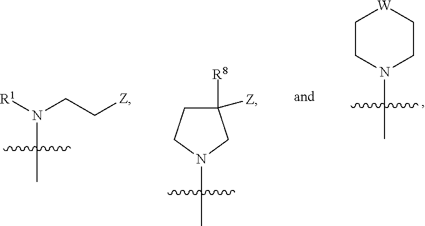

wherein
R¹ is H, C₁₋₆ alkyl, or C₄₋₁₄ alkyl-cycloalkyl;
Z is —OH, —SR^{7a}, —S(O)R^{7a}, —S(O)₂R^{7a}, —N(R^{7b}R^{7c}), or —N(R^{7b})C(O)R^{7c};
R^{7a}, R^{7b} and R^{7c} are each independently H or C₁₋₆ alkyl;
R⁸ is H or methyl;
W is —C(R^{7a})(Z); and
“ [symbol] ” indicates a point of attachment to the indole ring system.

Some embodiments of the compounds of Formula (X) are compounds having a Formula (I.a)

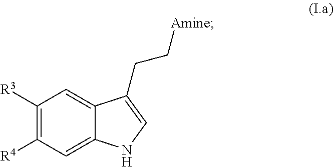

or a pharmaceutically acceptable salt or isomer thereof, wherein
R³ and R⁴ are each independently H, C₁₋₆ alkyl, or halogen;
“Amine” represents an amine that is

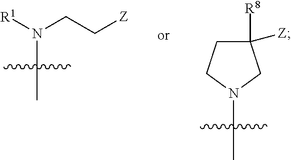

R¹ is H, C₁₋₆ alkyl, or C₄₋₁₄ alkyl-cycloalkyl;
R⁸ is H or methyl;
Z is —OH, —SR^{7a}, —S(O)R^{7a}, —S(O)₂R^{7a}, —N(R^{7b}R^{7c}), or —N(R^{7b})C(O)R^{7c}; and
R^{7a}, R^{7b} and R^{7c} are each independently H or C₁₋₆ alkyl.

Some embodiments of the compounds of Formula (X) are compounds having a Formula (V.a)

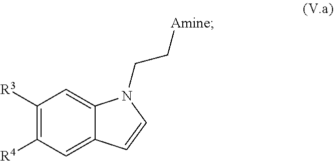

or a pharmaceutically acceptable salt or isomer thereof, wherein
R³ and R⁴ are each independently H, C₁₋₆ alkyl, or halogen;
“Amine” represents an amine that is

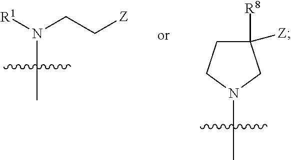

R¹ is H, C₁₋₆ alkyl, or C₄₋₁₄ alkyl-cycloalkyl;
R⁸ is H or methyl;
Z is —OH, —SR^{7a}, —S(O)R^{7a}, —S(O)₂R^{7a}, —N(R^{7b}R^{7c}), or —N(R^{7b})C(O)R^{7c}; and
R^{7a}, R^{7b} and R^{7c} are each independently H or C₁₋₆ alkyl.

Disclosed embodiments also include compositions comprising at least one compound according to the present invention, such as from 5% to 70% of at least one compound according to the present invention, at a dosage of from 0.1 mg to about 10,000 mg, more typically from about 25 mg to about 500 mg, and an excipient, an adjuvant, a carrier, or combinations thereof. Such compositions can further comprise at least one additional biologically active compound other than a compound or compounds according to the present invention. Exemplary excipients include magnesium carbonate, magnesium stearate, talc, pectin, dextrin, starch, tragacanth, cocoa butter, lactose, sucrose, mannitol, sorbitol, starch, methyl cellulose, hydroxypropyl-methylcellulose, sodium carboxymethylcellulose, gelatin, collagen, and any and all combinations thereof.

Compounds and/or compositions according to the present invention can be administered to a subject, including human subjects. One embodiment of the method may comprise administering a first dose of a compound or a composition according to the present invention, followed by administering at least a second dose. The composition may be formulated for administration to the subject multiple times per day, such as 1 to 3 times per day, and/or to the subject periodically, such as every 2 to 7 days. The compound and/or composition comprising at least one disclosed compound can be co-administered with a second active agent in a suitable weight ratio relative to the weight of the compound of from about 1:100 to about 100:1 (w/w).

The compound, or composition comprising the compound, may be administered for a variety of reasons, such as to increase neuronal plasticity, to treat a neurological disease, such as to treat a migraine, a headache, a cluster headache, post-traumatic stress disorder (PTSD), anxiety, depression, neurodegenerative disorder, Alzheimer's disease, Parkinson's disease, a psychological disorder, treatment resistant depression, suicidal ideation, a major depressive disorder, bipolar disorder, schizophrenia, stroke, traumatic brain injury, or addiction, or to treat a neuropsychiatric disease, such as a psychological disorder, treatment resistant depression, suicidal ideation, major depressive disorder, bipolar disorder, schizophrenia, post-traumatic stress disorder (PTSD), addiction, depression, or anxiety. The compound, or composition comprising at least one compound according to the invention, can also be administered to treat fibromyalgia, muscle pain, joint stiffness, osteoarthritis, rheumatoid arthritis, or muscle cramps, or to treat premenstrual dysphoric disorder (PMDD), premenstrual syndrome (PMS), post-partum depression, or menopause.

Certain disclosed embodiments concern administering a compound, or a composition comprising at least one compound according to the invention, to elicit a biological response by activating a 5-HT_{2A} receptor. For these embodiments, 5-HT_{2A} antagonist also can be administered in combination with at least one compound of the present invention to mitigate undesirable effects of 5-HT_{2A} agonism. The compound, or composition comprising at least one compound according to the invention, can be administered together with a serotonin receptor modulator, such as an agent selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), olanzapine, risperidone, pimavanserin, nelotanserin and lorcaserin. Other coadministration examples include administering at least one compound of the present invention, or composition comprising at least one compound according to the invention, together with one or more additional therapeutic agents selected from lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Pamate), diazepam (Valium), alprazolam (Xanax), or clonazepam (Klonopin). Certain disclosed embodiments also comprise administering at least one compound of the present invention, or composition comprising at least one compound according to the invention, together with an empathogenic agent, such as an agent selected from N-Allyl-3,4-methylenedioxy-amphetamine (MDAL), N-Butyl-3,4-methylenedioxyamphetamine (MDBU), N-Benzyl-3,4-methylenedioxyamphetamine (MDBZ), N-Cyclopropylmethyl-3,4-methylenedioxyamphetamine (MDCPM), N,N-Dimethyl-3,4-methylenedioxyamphetamine (MDDM), N-Ethyl-3,4-methylenedioxyamphetamine (MDE; MDEA), N-(2-Hydroxyethyl)-3,4-methylenedioxy amphetamine (MDHOET), N-Isopropyl-3,4-methylenedioxyamphetamine (MDIP), N-Methyl-3,4-ethylenedioxyamphetamine (MDMC), N-Methoxy-3,4-methylenedioxy amphetamine (MDMEO), N-(2-Methoxyethyl)-3,4-methylenedioxyamphetamine (MDMEOET) alpha,alpha,N-Trimethyl-3,4-methylenedioxyphenethylamine (MDMP; 3,4-Methylenedioxy-N-methylphentermine), N-Hydroxy-3,4-methylenedioxyamphetamine (MDOH), 3,4-Methylenedioxyphenethylamine (MDPEA), alpha,alpha-Dimethyl-3,4-methylenedioxyphenethylamine (MDPH; 3,4-methylenedioxyphentermine)N-Propargyl-3,4-methylenedioxyamphetamine (MDPL), Methylenedioxy-2-aminoindane (MDAI), N-methyl-1,3-benzodioxolylbutanamine (MBDB) 3,4-methylenedioxy-N-methyl-α-ethylphenylethylamine, 3,4-Methylenedioxyamphetamine MDA, Methylone (also known as “3,4-methylenedioxy-N-methylcathinone), Ethylone, also known as 3,4-methylenedioxy-N-ethylcathinone, GHB (Gamma Hydroxybutyrate), sodium oxybate, N-Propyl-3,4-methylenedioxyamphetamine (MDPR), or a combination thereof. Disclosed embodiments also concern administering at least one compound of the present invention, or composition comprising such compound, to increase at least one of translation, transcription, or secretion of neurotrophic factors.

A method for making disclosed compounds also is provided. Disclosed method for making embodiments include, for example, reacting an indole compound having a formula

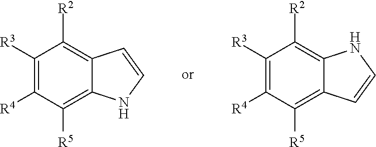

with an acylating agent to form a first intermediate comprising an acylated indole. The acylated indole is reacted with phthalimide, or a derivative thereof, followed by reaction with an amine reagent, or a protected amine, to form a second intermediate. The second intermediate is then used as is or may be converted into other desired compounds, where converting may comprise: forming an amino alcohol or an amino sulfide by treating an appropriate second intermediate with a hydride reducing agent; oxidizing an amino sulfide with a peracid to form an oxidized intermediate; reducing an oxidized intermediate with a hydride reducing agent to form a sulfone; forming an amino sulfoxide; forming a protected diamine and removing an amine protecting group to form a diamine; and acylating the diamine.

### BRIEF DESCRIPTION OF THE FIGURES

FIG. 1 shows control dose response curves for the selected GPCR Biosensor Assays.

### DETAILED DESCRIPTION

### I. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“Alkyl” refers to a straight or branched, saturated, aliphatic radical having the number of carbon atoms indicated. Alkyl can include any number of carbons, such as C₁₋₂, C₁₋₃, C₁₋₄, C₁₋₅, C₁₋₆, C₁₋₇, C₁₋₈, C₁₋₉, C₁₋₁₀, C₂₋₃, C₂₋₄, C₂₋₅, C₂₋₆, C₃₋₄, C₃₋₅, C₃₋₆, C₄₋₅, C₄₋₆ and C₅₋₆. For example, C₁₋₆ alkyl includes, but is not limited to, methyl, ethyl, propyl, isopropyl, butyl, isobutyl, sec-butyl, tert-butyl, pentyl, isopentyl, hexyl, etc. Alkyl groups can be substituted or unsubstituted.

“Alkylene” refers to a straight or branched, saturated, aliphatic radical having the number of carbon atoms indicated, and linking at least two other groups, i.e., a divalent hydrocarbon radical. The two moieties linked to the alkylene can be linked to the same atom or different atoms of the alkylene group. For instance, a straight chain alkylene can be the bivalent radical of —(CH₂)ₙ— where n is 1, 2, 3, 4, 5 or 6. Representative alkylene groups include, but are not limited to, methylene, ethylene, propylene, isopropylene, butylene, isobutylene, sec-butylene, pentylene and hexylene. Alkylene groups can be substituted or unsubstituted.

“Alkenyl” refers to a straight chain or branched hydrocarbon having at least 2 carbon atoms and at least one double bond. Alkenyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and C₆. Alkenyl groups can have any suitable number of double bonds, including, but not limited to, 1, 2, 3, 4, 5 or more. Examples of alkenyl groups include, but are not limited to, vinyl (ethenyl), propenyl, isopropenyl, 1-butenyl, 2-butenyl, isobutenyl, butadienyl, 1-pentenyl, 2-pentenyl, isopentenyl, 1,3-pentadienyl, 1,4-pentadienyl, 1-hexenyl, 2-hexenyl, 3-hexenyl, 1,3-hexadienyl, 1,4-hexadienyl, 1,5-hexadienyl, 2,4-hexadienyl, or 1,3,5-hexatrienyl. Alkenyl groups can be substituted or unsubstituted.

“Alkoxy” refers to an alkyl group having an oxygen atom that connects the alkyl group to the point of attachment: alkyl-O—. As for alkyl group, alkoxy groups can have any suitable number of carbon atoms, such as C₁₋₆. Alkoxy groups include, for example, methoxy, ethoxy, propoxy, iso-propoxy, butoxy, 2-butoxy, iso-butoxy, sec-butoxy, tert-butoxy, pentoxy, hexoxy, etc. The alkoxy groups can be further substituted with a variety of substituents described within. Alkoxy groups can be substituted or unsubstituted.

“Alkynyl” refers to either a straight chain or branched hydrocarbon having at least 2 carbon atoms and at least one triple bond. Alkynyl can include any number of carbons, such as C₂, C₂₋₃, C₂₋₄, C₂₋₅, C₂₋₆, C₂₋₇, C₂₋₈, C₂₋₉, C₂₋₁₀, C₃, C₃₋₄, C₃₋₅, C₃₋₆, C₄, C₄₋₅, C₄₋₆, C₅, C₅₋₆, and Ce. Examples of alkynyl groups include, but are not limited to, acetylenyl, propynyl, 1-butynyl, 2-butynyl, butadiynyl, 1-pentynyl, 2-pentynyl, isopentynyl, 1,3-pentadiynyl, 1,4-pentadiynyl, 1-hexynyl, 2-hexynyl, 3-hexynyl, 1,3-hexadiynyl, 1,4-hexadiynyl, 1,5-hexadiynyl, 2,4-hexadiynyl, or 1,3,5-hexatriynyl. Alkynyl groups can be substituted or unsubstituted.

“Amine” refers to an —N(R)2 group where the R groups can be hydrogen, alkyl, alkenyl, alkynyl, cycloalkyl, heterocycloalkyl, aryl, or heteroaryl, among others. The R groups can be the same or different. The amino groups can be primary (each R is hydrogen), secondary (one R is hydrogen) or tertiary (each R is other than hydrogen).

“Alkyl amine” refers to an alkyl group as defined within, having one or more amino groups. The amino groups can be primary, secondary or tertiary. The alkyl amine can be further substituted with a hydroxy group to form an amino-hydroxy group. Alkyl amines useful in the present invention include, but are not limited to, ethyl amine, propyl amine, isopropyl amine, ethylene diamine and ethanolamine. The amino group can link the alkyl amine to the point of attachment with the rest of the compound, be at the omega position of the alkyl group, or link together at least two carbon atoms of the alkyl group. One of skill in the art will appreciate that other alkyl amines are useful in the present invention.

“Aryl” refers to an aromatic ring system having any suitable number of ring atoms and any suitable number of rings. Aryl groups can include any suitable number of ring atoms, such as, 6, 7, 8, 9, 10, 11, 12, 13, 14, 15 or 16 ring atoms, as well as from 6 to 10, 6 to 12, or 6 to 14 ring members. Aryl groups can be monocyclic, fused to form bicyclic or tricyclic groups, or linked by a bond to form a biaryl group. Representative aryl groups include phenyl, naphthyl and biphenyl. Other aryl groups include benzyl, having a methylene linking group. Some aryl groups have from 6 to 12 ring members, such as phenyl, naphthyl or biphenyl. Other aryl groups have from 6 to 10 ring members, such as phenyl or naphthyl. Some other aryl groups have 6 ring members, such as phenyl. Aryl groups can be substituted or unsubstituted.

“Alkyl-aryl” refers to a radical having an alkyl component and an aryl component, where the alkyl component links the aryl component to the point of attachment. The alkyl component is as defined above, except that the alkyl component is at least divalent, an alkylene, to link to the aryl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₁₋₄, C₃₋₅, C₃₋₆, C₄₋₅, C₄₋₆ and C₅₋₆. In some instances, the alkyl component can be absent. The aryl component is as defined above. Examples of alkyl-aryl groups include, but are not limited to, benzyl and ethyl-benzene. Alkyl-aryl groups can be substituted or unsubstituted.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Cycloalkyl” refers to a saturated or partially unsaturated, monocyclic, bicyclic, fused bicyclic or bridged polycyclic ring assembly containing from 3 to 12 ring atoms, or the number of atoms indicated. Cycloalkyl can include any number of carbons, such as C₃₋₆, C₄₋₆, C₅₋₆, C₃₋₈, C₄₋₈, C₅₋₈, C₆₋₈, C₃₋₉, C₃₋₁₀, C₃₋₁₁, and C₃₋₁₂. Saturated monocyclic cycloalkyl rings include, for example, cyclopropyl, cyclobutyl, cyclopentyl, cyclohexyl, and cyclooctyl.

Bicyclic compounds include spirocyclic compounds, fused bicyclic compounds and bridged bicyclic compounds. Saturated bicyclic and polycyclic cycloalkyl rings include, for example, norbomane, [2.2.2] bicyclooctane, decahydronaphthalene and adamantane. Cycloalkyl groups can also be partially unsaturated, having one or more double or triple bonds in the ring. Representative cycloalkyl groups that are partially unsaturated include, but are not limited to, cyclobutene, cyclopentene, cyclohexene, cyclohexadiene (1,3- and 1,4-isomers), cycloheptene, cycloheptadiene, cyclooctene, cyclooctadiene (1,3-, 1,4- and 1,5-isomers), norbomene, and norbomadiene. When cycloalkyl is a saturated monocyclic C₃₋₈ cycloalkyl, exemplary groups include, but are not limited to cyclopropyl, cyclobutyl, cyclopentyl, cyclohexyl, cycloheptyl and cyclooctyl. When cycloalkyl is a saturated monocyclic C₃₋₆ cycloalkyl, exemplary groups include, but are not limited to cyclopropyl, cyclobutyl, cyclopentyl, and cyclohexyl. Cycloalkyl groups can be substituted or unsubstituted.

“Alkyl-cycloalkyl” refers to a radical having an alkyl component and a cycloalkyl component, where the alkyl component links the cycloalkyl component to the point of attachment. The alkyl component is as defined above, except that the alkyl component is at least divalent, an alkylene, to link to the cycloalkyl component and to the point of attachment. In some instances, the alkyl component can be absent. The alkyl component can include any number of carbons, such as C₁₋₆, C₁₋₂, C₁₋₃, C₁₋₄, C₁₋₅, C₃₋₆, C₂₋₄, C₂₋₅, C₂₋₆, C₃₋₄, C₃₋₅, C₃₋₆, C₄₋₅, C₄₋₆ and C₅₋₆. The cycloalkyl component is as defined within. Exemplary alkyl-cycloalkyl groups include, but are not limited to, methyl-cyclopropyl, methyl-cyclobutyl, methyl-cyclopentyl and methyl-cyclohexyl.

“Heterocycloalkyl” refers to a cycloalkyl as defined above, having from 3 to 12 ring members and from 1 to 4 heteroatoms of N, O and S. Heterocycloalkyl includes bicyclic compounds which include a heteroatom. Bicyclic compounds includes spirocyclic compounds, fused bicyclic compounds, and bridged bicyclic compounds The heteroatoms can also be oxidized, such as, but not limited to, —S(O)— and —S(O)₂—. Heterocycloalkyl groups can include any number of ring atoms, such as, 3 to 6, 4 to 6, 5 to 6, 3 to 8, 4 to 8, 5 to 8, 6 to 8, 3 to 9, 3 to 10, 3 to 11, or 3 to 12 ring members. Any suitable number of heteroatoms can be included in the heterocycloalkyl groups, such as 1, 2, 3, or 4, or 1 to 2, 1 to 3, 1 to 4, 2 to 3, 2 to 4, or 3 to 4. The heterocycloalkyl group can include groups such as aziridine, azetidine, pyrrolidine, piperidine, azepane, azocane, quinuclidine, pyrazolidine, imidazolidine, piperazine (1,2-, 1,3- and 1,4-isomers), oxirane, oxetane, tetrahydrofuran, oxane (tetrahydropyran), oxepane, thiirane, thietane, thiolane (tetrahydrothiophene), thiane (tetrahydrothiopyran), oxazolidine, isoxazolidine, thiazolidine, isothiazolidine, dioxolane, dithiolane, morpholine, thiomorpholine, dioxane, or dithiane. The heterocycloalkyl groups can also be fused to aromatic or non-aromatic ring systems to form members including, but not limited to, indoline. Heterocycloalkyl groups can be unsubstituted or substituted. For example, heterocycloalkyl groups can be substituted with C₁₋₆ alkyl or oxo (═O), among many others.

“Alkyl-heterocycloalkyl” refers to a radical having an alkyl component and a heterocycloalkyl component, where the alkyl component links the heterocycloalkyl component to the point of attachment. The alkyl component is as defined above, except that the alkyl component is at least divalent, an alkylene, to link to the heterocycloalkyl component and to the point of attachment. The alkyl component can include any number of carbons, such as C₀₋₆, C₁₋₂, C₁₋₃, C₁₋₄, C₁₋₅, C₁₋₆, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heterocycloalkyl component is as defined above. Alkyl-heterocycloalkyl groups can be substituted or unsubstituted.

“Halogen” refers to fluorine, chlorine, bromine and iodine.

“Haloalkyl” refers to alkyl, as defined above, where some or all of the hydrogen atoms are replaced with halogen atoms. As for alkyl group, haloalkyl groups can have any suitable number of carbon atoms, such as C₁₋₆. For example, haloalkyl includes trifluoromethyl, fluoromethyl, etc. In some instances, the term “perfluoro” can be used to define a compound or radical where all the hydrogens are replaced with fluorine. For example, perfluoromethyl refers to 1,1,1-trifluoromethyl.

“Haloalkoxy” refers to an alkoxy group where some or all of the hydrogen atoms are substituted with halogen atoms. As for an alkyl group, haloalkoxy groups can have any suitable number of carbon atoms, such as C₁₋₆. The alkoxy groups can be substituted with 1, 2, 3, or more halogens. When all the hydrogens are replaced with a halogen, for example by fluorine, the compounds are per-substituted, for example, perfluorinated. Haloalkoxy includes, but is not limited to, trifluoromethoxy, 2,2,2,-trifluoroethoxy, perfluoroethoxy, etc.

“Heteroaryl” refers to a monocyclic or fused bicyclic or tricyclic aromatic ring assembly containing 5 to 16 ring atoms, where from 1 to 5 of the ring atoms are a heteroatom such as N, O or S. Heteroaryl groups can include any number of ring atoms, such as, 5 to 6, 3 to 8, 4 to 8, 5 to 8, 6 to 8, 3 to 9, 3 to 10, 3 to 11, or 3 to 12 ring members. Any suitable number of heteroatoms can be included in the heteroaryl groups, such as 1, 2, 3, 4, or 5, or 1 to 2, 1 to 3, 1 to 4, 1 to 5, 2 to 3, 2 to 4, 2 to 5, 3 to 4, or 3 to 5. Heteroaryl groups can have from 5 to 8 ring members and from 1 to 4 heteroatoms, or from 5 to 8 ring members and from 1 to 3 heteroatoms, or from 5 to 6 ring members and from 1 to 4 heteroatoms, or from 5 to 6 ring members and from 1 to 3 heteroatoms. The heteroaryl group can include groups such as pyrrole, pyridine, imidazole, pyrazole, triazole, tetrazole, pyrazine, pyrimidine, pyridazine, triazine (1,2,3-, 1,2,4- and 1,3,5-isomers), thiophene, furan, thiazole, isothiazole, oxazole, and isoxazole. The heteroaryl groups can also be fused to aromatic ring systems, such as a phenyl ring, to form members including, but not limited to, benzopyrroles such as indole and isoindole, benzopyridines such as quinoline and isoquinoline, benzopyrazine (quinoxaline), benzopyrimidine (quinazoline), benzopyridazines such as phthalazine and cinnoline, benzothiophene, and benzofuran. Other heteroaryl groups include heteroaryl rings linked by a bond, such as bipyridine. Heteroaryl groups can be substituted or unsubstituted.

“Alkyl-heteroaryl” refers to a radical having an alkyl component and a heteroaryl component, where the alkyl component links the heteroaryl component to the point of attachment. The alkyl component is as defined above, except that the alkyl component is at least divalent, an alkylene, to link to the heteroaryl component and to the point of attachment. The alkyl component can include any number of carbons, such as C₀₋₆, C₁₋₂, C₁₋₃, C₁₋₄, C₁₋₅, C₁₋₄, C₂₋₃, C₂₋₄, C₂₋₅, C₂₋₆, C₃₋₄, C₃₋₅, C₃₋₆, C₄₋₅, C₄₋₆ and C₅₋₆. In some instances, the alkyl component can be absent. The heteroaryl component is as defined within. Alkyl-heteroaryl groups can be substituted or unsubstituted.

“Isomers” refers to compounds with same chemical formula but different connectivity between the atoms in the molecule, leading to distinct chemical structures. Isomers include structural isomers and stereoisomers. Examples of structural isomers include, but are not limited to tautomers and regioisomers. Examples of stereoisomers include but are not limited to diastereomers and enantiomers.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of ordinary skill in the art will recognize that other pharmaceutical excipients are useful in the present invention.

“Salt” refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (fumaric acid, acetic acid, propionic acid, glutamic acid, citric acid and the like) salts, quaternary ammonium (methyl iodide, ethyl iodide, and the like) salts. A person of ordinary skill in the art will appreciate that pharmaceutically acceptable salts are generally non-toxic. Additional information on suitable pharmaceutically acceptable salts can be found in Remington's Pharmaceutical Sciences, 17th ed., Mack Publishing Company, Easton, Pa., 1985, which is incorporated herein by reference. Neutral forms of disclosed compound embodiments may be regenerated by contacting the salt with a base or acid and isolating the parent compound in a conventional manner. The parent form of the compound differs from the various salt forms in certain physical properties, such as solubility in polar solvents, but otherwise the salts are equivalent to the parent form of the compound for the purposes of the present invention.

“Pharmaceutically acceptable salt” refers to a compound in salt form, wherein the compound are suitable for administration to a subject. Representative pharmaceutically acceptable salts include salts of acetic, ascorbic, benzenesulfonic, benzoic, camphorsulfonic, citric, ethanesulfonic, edisylic, fumaric, gentisic, gluconic, glucoronic, glutamic, hippuric, hydrobromic, hydrochloric, isethionic, lactic, lactobionic, maleic, malic, mandelic, methanesulfonic, mucic, naphthalenesulfonic, naphthalene-1,5-disulfonic, naphthalene-2,6-disulfonic, nicotinic, nitric, orotic, pamoic, pantothenic, phosphoric, succinic, sulfuric, tartaric, p-toluenesulfonic and xinafoic acid, and the like.

### II. Compounds

The present invention includes all tautomers and stereoisomers of compounds of the present invention, either in admixture or in pure or substantially pure form. The compounds of the present invention can have asymmetric centers, particularly at carbon atoms, and therefore the compounds of the present invention can exist in diastereomeric or enantiomeric forms or mixtures thereof. All conformational isomers (e.g., cis and trans isomers) and all optical isomers (e.g., enantiomers and diastereomers), racemic, diastereomeric and other mixtures of such isomers, as well as solvates, hydrates, isomorphs, polymorphs and tautomers, are within the scope of the present invention.

Compounds according to the present invention can be prepared using diastereomers, enantiomers or racemic mixtures as starting materials. Furthermore, diastereomer and enantiomer products can be separated by chromatography, fractional crystallization or other methods known to those of or ordinary skill in the art.

Several aspects of the present disclosure include compounds having Formula (X) shown below:

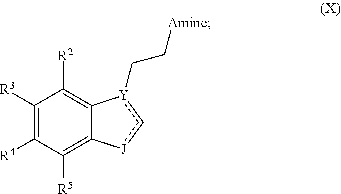

or a pharmaceutically acceptable salt or isomer thereof. Formula (X) includes an indole ring system and does not include tryptamine.

With reference to Formula (X), J and Y are each independently C, CH, N, or NH, one “ [symbol] ” is a single bond, and one “ [symbol] ” is a double bond. In several embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6d}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl. In further embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, or C₁₋₆ haloalkoxy. In further embodiments, R², R³, R⁴ and R⁵ are each independently H, C₁₋₆ alkyl, C₂₋₆ alkenyl, C₂₋₆ alkynyl, or halogen. In further embodiments, R², R³, R⁴ and R⁵ are each independently H, C₁₋₆ alkyl, or halogen. In further embodiments, at least one of R², R³, R⁴ and R⁵ is halogen (e.g., F). In further embodiments, one of R², R³, R⁴ and R⁵ is halogen (e.g., F). In further embodiments, one of R³ and R⁴ is halogen (e.g., F). In further embodiments, two of R², R³, R⁴ and R⁵ are halogen (e.g., F). In further embodiments, R¹ and R² are halogen (e.g., F), R¹ and R³ are halogen (e.g., F), R¹ and R⁴ are halogen (e.g., F), R² and R³ are halogen (e.g., F), R² and R⁴ are halogen (e.g., F), or R³ and R⁴ are halogen (e.g., F). In further embodiments, three of R², R³, R⁴ and R⁵ are halogen (e.g., F). In further embodiments, R¹, R² and R³ are halogen (e.g., F), R², R³ and R⁴ are halogen (e.g., F), R¹, R² and R⁴ are halogen (e.g., F), or R¹, R³ and R⁴ are halogen (e.g., F). In further embodiments, R², R³, R⁴ and R⁵ are halogen (e.g., F). Alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl. In several embodiments, R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In several embodiments, R^{6b}, R^{6c} and R^{6d} are each independently H or C₁₋₆ alkyl. “Amine” can be an aliphatic amine, such as an alkyl amine or substituted alkyl amine, or a heterocyclic amine with the amine nitrogen forming part of the ring system. In several embodiments, “Amine” represents an amine selected from

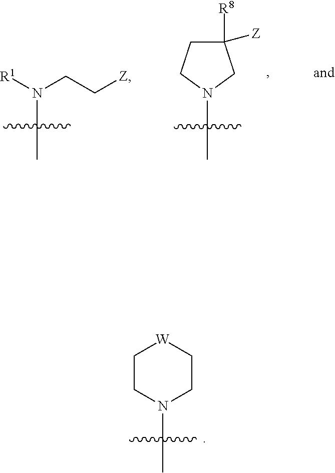

In several embodiments, R¹ is H, C₁₋₆ alkyl, or C₄₋₁₄ alkyl-cycloalkyl. In several embodiments, Z is —OH, —SR^{7a}, —S(O)R^{7a}, —S(O)₂R^{7a}, —N(R^{7b}R^{7c}), or —N(R^{7b})C(O)R^{7c}. In several embodiments, R^{7a}, R^{7b} and R^{7c} are each independently H or C₁₋₆ alkyl. In several embodiments, R⁸ is H or methyl. In several embodiments, W is —C(R^{7a})(Z). In several embodiments, “ [symbol] ” indicates a point of attachment to the indole ring system, such as an ethylene (—CH₂CH₂—) moiety at the 3 position of the indole ring system.

The compounds of the present disclosure exclude, or do otherwise not include, the compounds

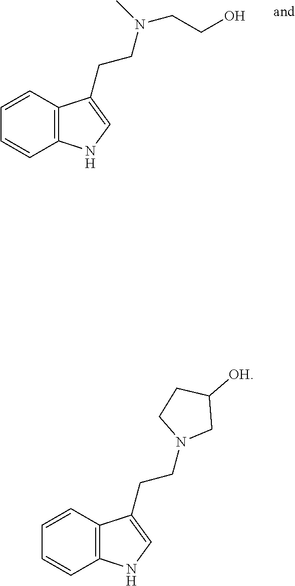 [SMILES: CN(CCO)CCc1c[nH]c2ccccc12 ; OC1CCN(CCc2c[nH]c3ccccc23)C1]

Some embodiments of the compounds of Formula (X) include compounds having Formula (I):

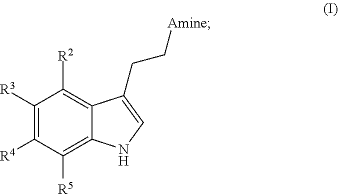

or a pharmaceutically acceptable salt or isomer thereof. Formula (I) includes an indole ring system and does not include tryptamine.

With reference to Formula (I), R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl. In further embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, or C₁₋₆ haloalkoxy. In further embodiments, R², R³, R⁴ and R⁵ are each independently H, C₁₋₆ alkyl, C₂₋₆ alkenyl, C₂₋₆ alkynyl, or halogen. In further embodiments, R², R³, R⁴ and R⁵ are each independently H, C₁₋₆ alkyl, or halogen. In further embodiments, at least one of R², R³, R⁴ and R⁵ is halogen (e.g., F). In further embodiments, one of R², R³, R⁴ and R⁵ is halogen (e.g., F). In further embodiments, one of R³ and R⁴ is halogen (e.g., F). In further embodiments, two of R², R³, R⁴ and R⁵ are halogen (e.g., F). In further embodiments, R¹ and R² are halogen (e.g., F), R¹ and R³ are halogen (e.g., F), R¹ and R⁴ are halogen (e.g., F), R² and R³ are halogen (e.g., F), R² and R⁴ are halogen (e.g., F), or R³ and R⁴ are halogen (e.g., F). In further embodiments, three of R², R³, R⁴ and R⁵ are halogen (e.g., F). In further embodiments, R¹, R² and R³ are halogen (e.g., F), R², R³ and R⁴ are halogen (e.g., F), R¹, R² and R⁴ are halogen (e.g., F), or R¹, R³ and R⁴ are halogen (e.g., F). In further embodiments, R², R³, R⁴ and R⁵ are halogen (e.g., F). Alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl. In several embodiments, R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In several embodiments, R^{6b}, R^{6c} and R^{6d} are each independently H or C₁₋₆ alkyl. “Amine” represents an amine selected from

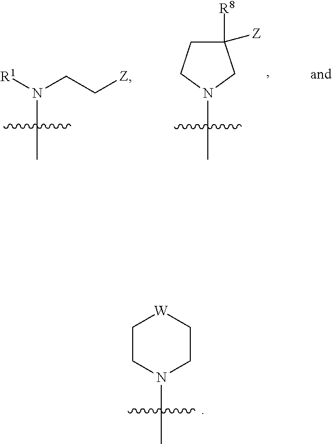

In several embodiments, R¹ is H, C₁₋₆ alkyl, or C₄₋₁₄ alkyl-cycloalkyl. In several embodiments, Z is —OH, —SR^{7a}, —S(O)R^{7a}, —S(O)₂R^{7a}, —N(R^{7b}R^{7c}), or —N(R^{7b})C(O)R^{7c}. In several embodiments, R^{7a}, R^{7b} and R^{7c} are each independently H or C₁₋₆ alkyl. In several embodiments, R⁸ is H or methyl. In several embodiments, W is —C(R^{7a})(Z). In several embodiments, “ [symbol] ” indicates a point of attachment to the indole ring system.

In several embodiments related to compounds having Formula (I), the amine is

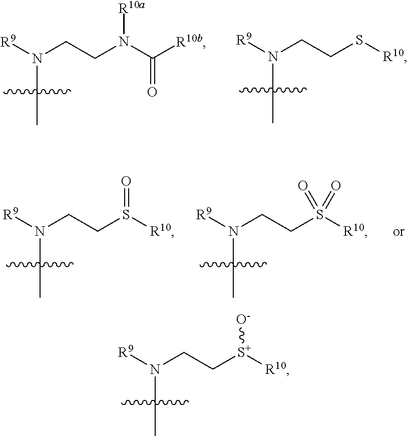

and R⁹, R¹⁰, R^{10a}, and R^{10b} are each independently selected from H, C₁₋₆ alkyl, and C₄₋₁₄ alkyl-cycloalkyl.

In several embodiments related to compounds having Formula (I), the amine is

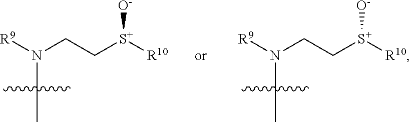

and R⁹ and R¹⁰ are each independently selected from H, C₁₋₆ alkyl, and C₄₋₁₄ alkyl-cycloalkyl.

In several embodiments related to compounds having Formula (I), R¹⁰, R^{10a}, and R^{10b} are methyl.

In several embodiments related to compounds having Formula (I), the amine has a formula

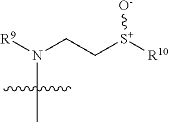

and R⁹ and R¹⁰ are each independently selected from C₁₋₆ alkyl.

In several embodiments related to compounds having Formula (I), the amine has a formula

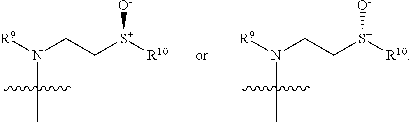

In several embodiments related to compounds having Formula (I), the amine is

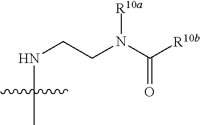

and R^{10a} and R^{10b} are each independently selected from H, C₁₋₆ alkyl, and C₄₋₁₄ alkyl-cycloalkyl.

In several embodiments related to compounds having Formula (I), R^{10a} and R^{10b} are methyl.

In several embodiments, the compound having Formula (I) is

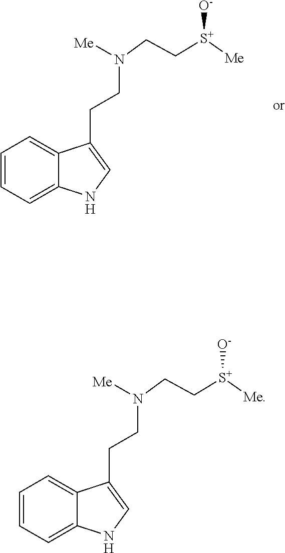 [SMILES: CN(CCc1c[nH]c2ccccc12)CC[S@+](C)[O-] ; CN(CCc1c[nH]c2ccccc12)CC[S@@+](C)[O-]]

In several embodiments related to compounds having Formula (I), the amine is

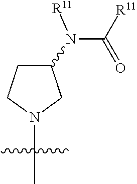

and each R¹¹ independently is H or C₁₋₆ alkyl.

In several embodiments related to compounds having Formula (I), the amine is

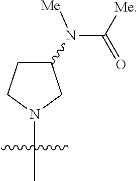 [SMILES: *N1CCC(N(C)C(C)=O)C1]

In several embodiments related to compounds having Formula (I), the amine is

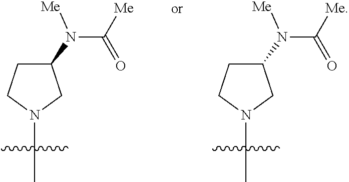 [SMILES: *N1CC[C@@H](N(C)C(C)=O)C1 ; *N1CC[C@H](N(C)C(C)=O)C1]

In several embodiments, the compound having Formula (I) is

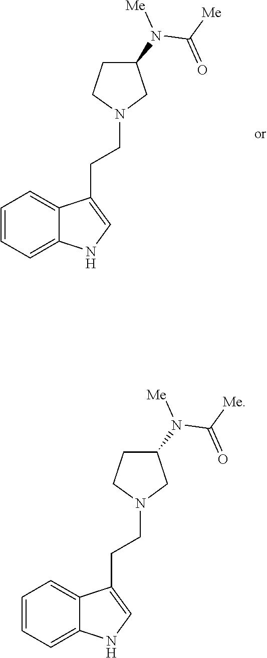 [SMILES: CC(=O)N(C)[C@@H]1CCN(CCc2c[nH]c3ccccc23)C1 ; CC(=O)N(C)[C@H]1CCN(CCc2c[nH]c3ccccc23)C1]

In several embodiments related to compounds having Formula (I), the amine is

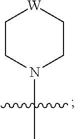

W is —C(R^{7a})(Z); R^{7a} is H or C₁₋₆ alkyl; and Z is —OH.

In several embodiments related to compounds having Formula (I), the amine is

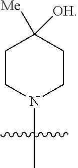 [SMILES: *N1CCC(C)(O)CC1]

In several embodiments, the compound having Formula (I) is

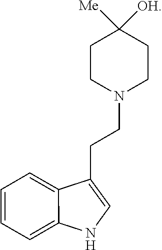 [SMILES: CC1(O)CCN(CCc2c[nH]c3ccccc23)CC1]

Some embodiments of the compounds of Formula (X) include compounds of Formula (II):

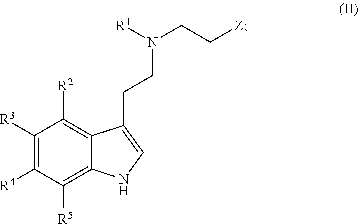

or a pharmaceutically acceptable salt or isomer thereof.

With reference to Formula (II), R¹ is H, C₁₋₆ alkyl, or C₄₋₁₄ alkyl-cycloalkyl. In several embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In further embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, or C₁₋₆ haloalkoxy. In further embodiments, R², R³, R⁴ and R⁵ are each independently H, C₁₋₆ alkyl, C₂₋₆ alkenyl, C₂₋₆ alkynyl, or halogen. In further embodiments, R², R³, R⁴ and R⁵ are each independently H, C₁₋₆ alkyl, or halogen. In further embodiments, at least one of R², R³, R⁴ and R⁵ is halogen (e.g., F). In further embodiments, one of R², R³, R⁴ and R⁵ is halogen (e.g., F). In further embodiments, one of R³ and R⁴ is halogen (e.g., F). In further embodiments, two of R², R³, R⁴ and R⁵ are halogen (e.g., F). In further embodiments, R¹ and R² are halogen (e.g., F), R¹ and R³ are halogen (e.g., F), R¹ and R⁴ are halogen (e.g., F), R² and R³ are halogen (e.g., F), R² and R⁴ are halogen (e.g., F), or R³ and R⁴ are halogen (e.g., F). In further embodiments, three of R², R³, R⁴ and R are halogen (e.g., F). In further embodiments, R¹, R² and R³ are halogen (e.g., F), R², R³ and R⁴ are halogen (e.g., F), R¹, R² and R⁴ are halogen (e.g., F), or R¹, R³ and R⁴ are halogen (e.g., F). In further embodiments, R², R³, R⁴ and R⁵ are halogen (e.g., F). Alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl. In several embodiments, R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl. In several embodiments, R^{6b}, R^{6c} and R^{6d} are each independently H or C₁₋₆ alkyl. In several embodiments, Z is —OH, —SR^{7a}, —S(O)R^{7a}, —S(O)₂R^{7a}, —N(R^{7b}R^{7c}), or —N(R^{7b})C(O)R^{7c}. In several embodiments, R^{7a}, R^{7b} and R^{7c} are each independently H or C₁₋₆ alkyl.

In several embodiments related to compounds having Formula (II), R¹ is methyl, Z is —S(O)R^{7a}, R^{7a} is methyl, and R², R³, R⁴ and R⁵ are H.

Some embodiments of the compounds of Formula (X) include compounds of Formula (III):

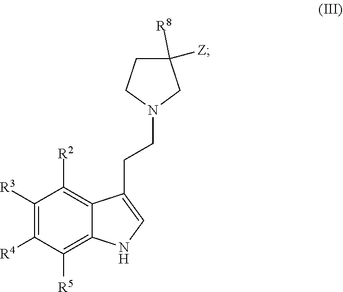

or a pharmaceutically acceptable salt or isomer thereof.

With reference to Formula (III), R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In further embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, or C₁₋₆ haloalkoxy. In further embodiments, R², R³, R⁴ and R⁵ are each independently H, C₁₋₆ alkyl, C₂₋₆ alkenyl, C₂₋₆ alkynyl, or halogen. In further embodiments, R², R³, R⁴ and R⁵ are each independently H, C₁₋₆ alkyl, or halogen. In further embodiments, at least one of R², R³, R⁴ and R⁵ is halogen (e.g., F). In further embodiments, one of R², R³, R⁴ and R⁵ is halogen (e.g., F). In further embodiments, one of R³ and R⁴ is halogen (e.g., F). In further embodiments, two of R², R³, R⁴ and R⁵ are halogen (e.g., F). In further embodiments, R¹ and R² are halogen (e.g., F), R¹ and R³ are halogen (e.g., F), R¹ and R⁴ are halogen (e.g., F), R² and R³ are halogen (e.g., F), R² and R⁴ are halogen (e.g., F), or R³ and R⁴ are halogen (e.g., F). In further embodiments, three of R², R³, R⁴ and R⁵ are halogen (e.g., F). In further embodiments, R¹, R² and R³ are halogen (e.g., F), R², R³ and R⁴ are halogen (e.g., F), R¹, R² and R⁴ are halogen (e.g., F), or R¹, R³ and R⁴ are halogen (e.g., F). In further embodiments, R², R³, R⁴ and R⁵ are halogen (e.g., F). Alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl. In several embodiments, R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In several embodiments, R^{6b}, R^{6c} and R^{6d} are each independently H or C₁₋₆ alkyl. In several embodiments, R⁸ is H or methyl. In several embodiments, Z is —OH, —SR^{7a}, —S(O)R^{7a}, —S(O)₂R^{7a}, —N(R^{7b}R^{7c}), or —N(R^{7b})C(O)R^{7c}. In several embodiments, R^{7a}, R^{7b} and R^{7c} are each independently H or C₁₋₆ alkyl.

In several embodiments related to compounds having Formula (III), Z is —N(R^{7b})C(O)R^{7c}, R^{7b} and R^{7c} are Me, and R², R³, R⁴ and R⁵ are H.

In several embodiments, the compound having Formula (III), is

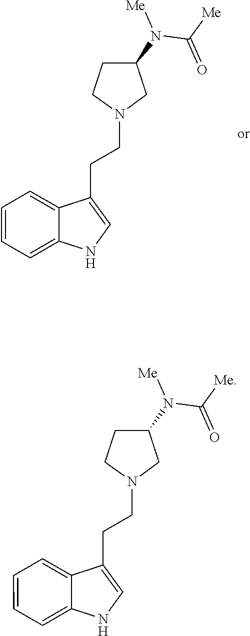 [SMILES: CC(=O)N(C)[C@@H]1CCN(CCc2c[nH]c3ccccc23)C1 ; CC(=O)N(C)[C@H]1CCN(CCc2c[nH]c3ccccc23)C1]

Some embodiments of the compounds of Formula (X) include compounds of Formula (IV):

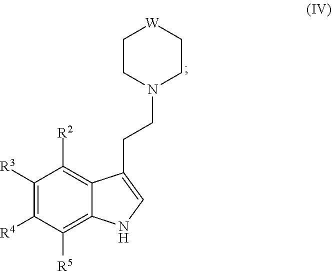

or a pharmaceutically acceptable salt or isomer thereof.

With reference to Formula (IV), R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In further embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, or C₁₋₆ haloalkoxy. In further embodiments, R², R³, R⁴ and R⁵ are each independently H, C₁₋₆ alkyl, C₂₋₆ alkenyl, C₂₋₆ alkynyl, or halogen. In further embodiments, R², R³, R⁴ and R⁵ are each independently H, C₁₋₆ alkyl, or halogen. In further embodiments, at least one of R², R³, R⁴ and R⁵ is halogen (e.g., F). In further embodiments, one of R², R³, R⁴ and R⁵ is halogen (e.g., F). In further embodiments, one of R³ and R⁴ is halogen (e.g., F). In further embodiments, two of R², R³, R⁴ and R⁵ are halogen (e.g., F). In further embodiments, R¹ and R² are halogen (e.g., F), R¹ and R³ are halogen (e.g., F), R¹ and R⁴ are halogen (e.g., F), R² and R³ are halogen (e.g., F), R² and R⁴ are halogen (e.g., F), or R³ and R⁴ are halogen (e.g., F). In further embodiments, three of R², R³, R⁴ and R⁵ are halogen (e.g., F). In further embodiments, R¹, R² and R³ are halogen (e.g., F), R², R³ and R⁴ are halogen (e.g., F), R¹, R² and R⁴ are halogen (e.g., F), or R¹, R³ and R⁴ are halogen (e.g., F). In further embodiments, R², R³, R⁴ and R⁵ are halogen (e.g., F). Alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl. In several embodiments, R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In several embodiments, R^{6b}, R^{6c} and R^{6d} are each independently H or C₁₋₆ alkyl. In several embodiments, W is —C(R^{7a})(Z). In several embodiments, Z is OH, S, —S(O), —S(O)₂, —N(R^{7a}), or —NC(O)R^{7a}. In several embodiments, R^{7a} is H or C₁₋₆ alkyl.

In several embodiments related to compounds having Formula (IV), W is —C(R^{7a})(OH), R^{7a} is methyl and R², R³, R⁴ and R⁵ are H.

In several embodiments, the compound having Formula (IV), is

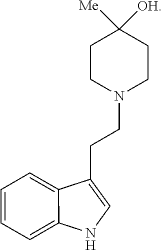 [SMILES: CC1(O)CCN(CCc2c[nH]c3ccccc23)CC1]

Some embodiments of the compounds of Formula (X) include compounds of Formula (V):

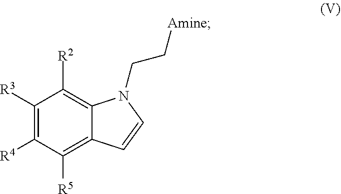

or a pharmaceutically acceptable salt or isomer thereof. Formula (V) includes an indole ring system and does not include tryptamine.

With reference to Formula (V), R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In further embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, or C₁₋₆ haloalkoxy. In further embodiments, R², R³, R⁴ and R⁵ are each independently H, C₁₋₆ alkyl, C₂₋₆ alkenyl, C₂₋₆ alkynyl, or halogen. In further embodiments, R², R³, R⁴ and R⁵ are each independently H, C₁₋₆ alkyl, or halogen. In further embodiments, at least one of R², R³, R⁴ and R⁵ is halogen (e.g., F). In further embodiments, one of R², R³, R⁴ and R⁵ is halogen (e.g., F). In further embodiments, one of R³ and R⁴ is halogen (e.g., F). In further embodiments, two of R², R³, R⁴ and R⁵ are halogen (e.g., F). In further embodiments, R¹ and R² are halogen (e.g., F), R¹ and R³ are halogen (e.g., F), R¹ and R⁴ are halogen (e.g., F), R² and R³ are halogen (e.g., F), R² and R⁴ are halogen (e.g., F), or R³ and R⁴ are halogen (e.g., F). In further embodiments, three of R², R³, R⁴ and R⁵ are halogen (e.g., F). In further embodiments, R¹, R² and R³ are halogen (e.g., F), R², R³ and R⁴ are halogen (e.g., F), R¹, R² and R⁴ are halogen (e.g., F), or R¹, R³ and R⁴ are halogen (e.g., F). In further embodiments, R², R³, R⁴ and R⁵ are halogen (e.g., F). Alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl. In several embodiments, R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In several embodiments, R^{6b}, R^{6c} and R^{6d} are each independently H or C₁₋₆ alkyl. “Amine” represents an amine selected from

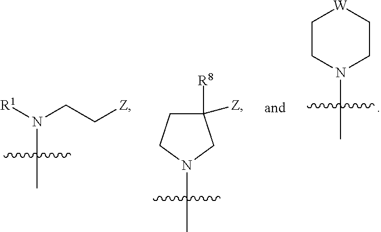

In several embodiments, R¹ is H, C₁₋₆ alkyl, or C₄₋₁₄ alkyl-cycloalkyl. In several embodiments, Z is —OH, —SR^{7a}, —S(O)R^{7a}, —S(O)₂R^{7a}, —N(R^{7b}R^{7c}), or —N(R^{7b})C(O)R^{7c}. In several embodiments, R^{7a}, R^{7b} and R^{7c} are each independently H or C₁₋₆ alkyl. In several embodiments, R⁸ is H or methyl. In several embodiments, W is —C(R^{7a})(Z). In several embodiments, “ [symbol] ” indicates a point of attachment to the indole ring system.

Certain embodiments of the compounds of Formula (X) include compounds of Formula (I.a)

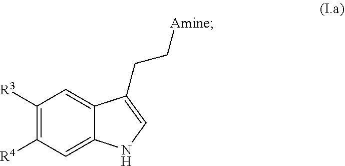

or a pharmaceutically acceptable salt or isomer thereof.

With reference to Formula (I.a), R³ and R⁴ are each independently H, C₁₋₆ alkyl, or halogen. In several embodiments, “Amine” represents an amine that is

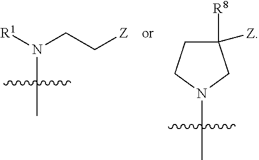

In several embodiments, R¹ is H, C₁₋₆ alkyl, or C₄₋₁₄ alkyl-cycloalkyl. In several embodiments, R⁸ is H or methyl. In several embodiments, Z is —OH, —SR^{7a}, —S(O)R^{7a}, —S(O)₂R^{7a}, —N(R^{7b}R^{7c}), or —N(R^{7b})C(O)R^{7c}. In several embodiments, R^{7a}, R^{7b} and R^{7c} are each independently H or C₁₋₆ alkyl.

In several embodiments related to compounds having Formula (I.a), R³ and R⁴ are each independently H or halogen, the amine is

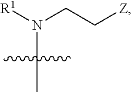

R¹ is H or methyl, Z is —OH or S(O)₂R^{7a}, and R^{7a} is H or C₁₋₆ alkyl.

In several embodiments related to compounds having Formula (I.a), R³ and R⁴ are each independently H or halogen, the amine is

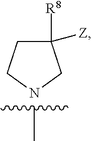

R⁸ is H or methyl, and Z is —OH.

In several embodiments related to compounds having Formula (I.a), R³ and R⁴ are each independently H or F, R¹ is methyl, and R⁸ is H or methyl.

In several embodiments related to compounds having Formula (I.a), R³ is H and R⁴ is F, R³ is F and R⁴ is H, or R³ and R⁴ are F. In some embodiments, R³ is H and R⁴ is F. In some embodiments, R³ is F and R⁴ is H. In some embodiments, R³ and R⁴ are F.

Certain embodiments of the compounds of Formula (X) include compounds of Formula (V.a)

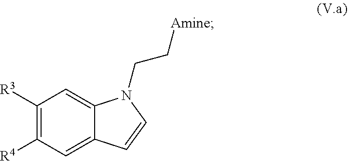

or a pharmaceutically acceptable salt or isomer thereof.

With reference to Formula (V.a), R³ and R⁴ are each independently H, C₁₋₆ alkyl, or halogen. In several embodiments, “Amine” represents an amine that is

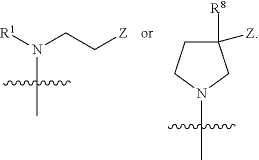

In several embodiments, R¹ is H, C₁₋₆ alkyl, or C₄₋₁₄ alkyl-cycloalkyl. In several embodiments, R⁸ is H or methyl. In several embodiments, Z is —OH, —SR^{7a}, —S(O)R^{7a}, —S(O)₂R^{7a}, —N(R^{7b}R^{7c}), or —N(R^{7b})C(O)R^{7c}. In several embodiments, R^{7a}, R^{7b} and R^{7c} are each independently H or C₁₋₆ alkyl.

In several embodiments related to compounds having Formula (V.a), R³ and R⁴ are each independently H or halogen, the amine is

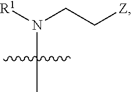

R¹ is H or methyl, Z is —OH or S(O)₂R^{7a}, and R^{7a} is H or C₁₋₆ alkyl.

In several embodiments related to compounds having Formula (V.a), R³ and R⁴ are each independently H or halogen, the amine is

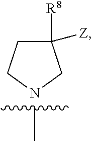

R⁸ is H or methyl, and Z is —OH.

In several embodiments related to compounds having Formula (V.a), R³ and R⁴ are each independently H or F, R is methyl, and R⁸ is H or methyl.

In several embodiments related to compounds having Formula (V.a), R³ is H and R⁴ is F, R³ is F and R⁴ is H, or R³ and R⁴ are F. In some embodiments, R³ is H and R⁴ is F. In some embodiments, R³ is F and R⁴ is H. In some embodiments, R³ and R⁴ are F.

In some embodiments of any of the Formulae described herein (i.e., Formula I, II, III, IV, V, I.a, or V.a) as applicable, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl. In further embodiments, R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, or C₁₋₆ haloalkoxy. In further embodiments, R², R³, R⁴ and R⁵ are each independently H, C₁₋₆ alkyl, C₂₋₆ alkenyl, C₂₋₆ alkynyl, or halogen. In further embodiments, R², R³, R⁴ and R⁵ are each independently H, C₁₋₆ alkyl, or halogen. In further embodiments, at least one of R², R³, R⁴ and R⁵ is halogen (e.g., F). In further embodiments, one of R², R³, R⁴ and R⁵ is halogen (e.g., F). In further embodiments, one of R³ and R⁴ is halogen (e.g., F). In further embodiments, two of R², R³, R⁴ and R⁵ are halogen (e.g., F). In further embodiments, R¹ and R² are halogen (e.g., F), R¹ and R³ are halogen (e.g., F), R¹ and R⁴ are halogen (e.g., F), R² and R³ are halogen (e.g., F), R² and R⁴ are halogen (e.g., F), or R³ and R⁴ are halogen (e.g., F). In further embodiments, three of R², R³, R⁴ and R⁵ are halogen (e.g., F). In further embodiments, R¹, R² and R³ are halogen (e.g., F), R², R³ and R⁴ are halogen (e.g., F), R¹, R² and R⁴ are halogen (e.g., F), or R¹, R³ and R⁴ are halogen (e.g., F). In further embodiments, R², R³, R⁴ and R⁵ are halogen (e.g., F).

In several embodiments, the compound of the present disclosure is (S)—N-(2-(1H-indol-3-yl)ethyl)-N-methyl-2-(methylsulfinyl)ethan-1-amine, (R)—N-(2-(1H-indol-3-yl)ethyl)-N-methyl-2-(methylsulfinyl)ethan-1-amine, (R)—N-(1-(2-(1H-indol-3-yl)ethyl)pyrrolidin-3-yl)-N-methylacetamide, (S)—N-(1-(2-(1H-indol-3-yl)ethyl)pyrrolidin-3-yl)-N-methylacetamide, 1-(2-(1H-indol-3-yl)ethyl)-4-methylpiperidin-4-ol, or a pharmaceutically acceptable salt thereof.

In several embodiments, the compound of the present disclosure is:

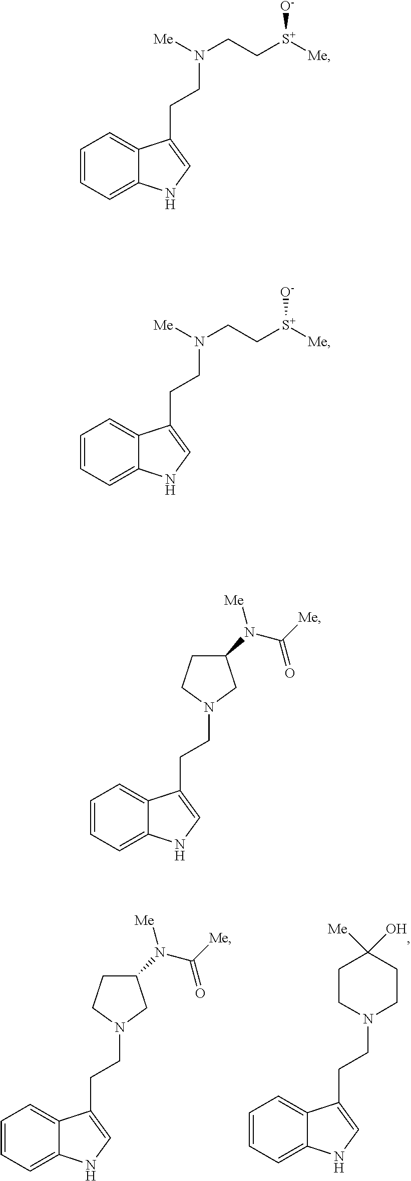 [SMILES: CN(CCc1c[nH]c2ccccc12)CC[S@+](C)[O-] ; CN(CCc1c[nH]c2ccccc12)CC[S@@+](C)[O-] ; CC(=O)N(C)[C@@H]1CCN(CCc2c[nH]c3ccccc23)C1 ; CC(=O)N(C)[C@H]1CCN(CCc2c[nH]c3ccccc23)C1 ; CC1(O)CCN(CCc2c[nH]c3ccccc23)CC1]

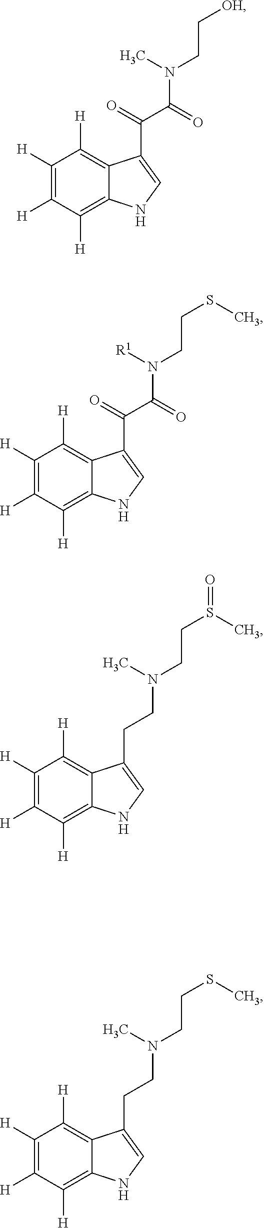

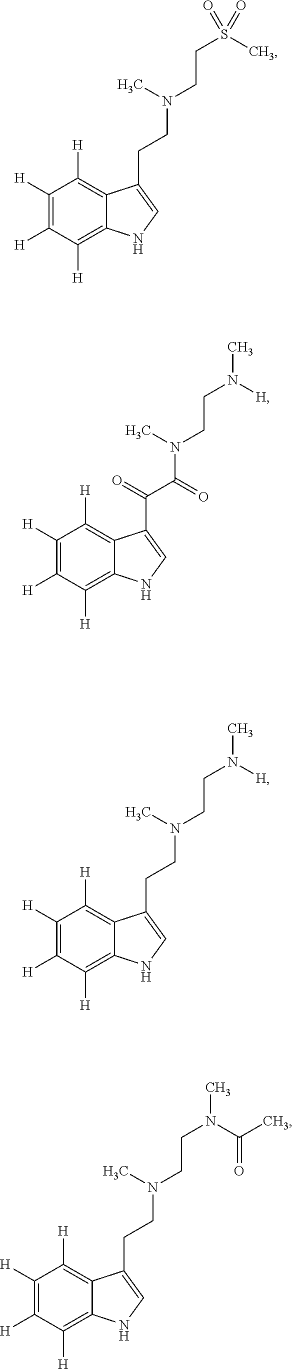

or a pharmaceutically acceptable salt thereof.

In several embodiments, the compound of the present disclosure is provided in TABLE 1.

TABLE 1

| Cpd | Structure |
|---|---|
| 1 | 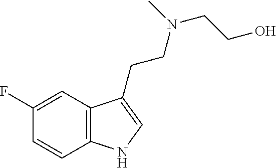 [SMILES: CN(CCO)CCc1c[nH]c2ccc(F)cc12] |
| 2 | 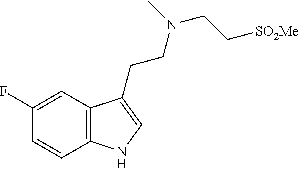 [SMILES: CN(CCc1c[nH]c2ccc(F)cc12)CCS(C)(=O)=O] |
| 3 | 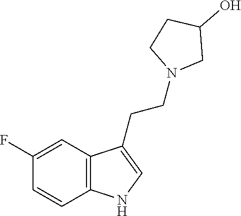 [SMILES: OC1CCN(CCc2c[nH]c3ccc(F)cc23)C1] |
| 4 | 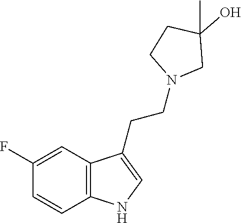 [SMILES: CC1(O)CCN(CCc2c[nH]c3ccc(F)cc23)C1] |
| 5 | 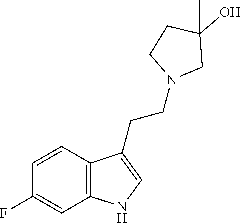 [SMILES: CC1(O)CCN(CCc2c[nH]c3cc(F)ccc23)C1] |
| 6 | 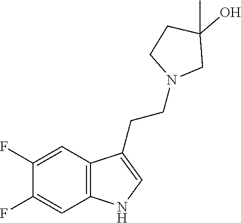 [SMILES: CC1(O)CCN(CCc2c[nH]c3cc(F)c(F)cc23)C1] |
| 7 | 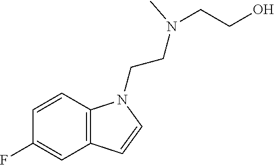 [SMILES: CN(CCO)CCn1ccc2cc(F)ccc21] |
| 8 | 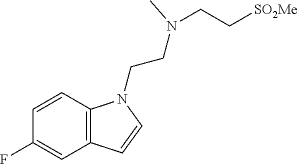 [SMILES: CN(CCn1ccc2cc(F)ccc21)CCS(C)(=O)=O] |
| 9 | 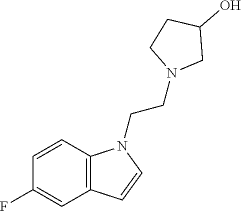 [SMILES: OC1CCN(CCn2ccc3cc(F)ccc32)C1] |
| 10 | 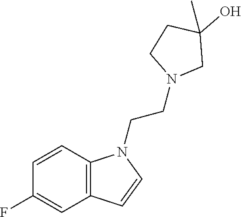 [SMILES: CC1(O)CCN(CCn2ccc3cc(F)ccc32)C1] |
| 11 | 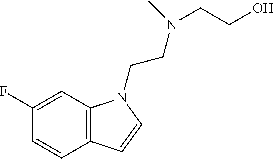 [SMILES: CN(CCO)CCn1ccc2ccc(F)cc21] |
| 12 | 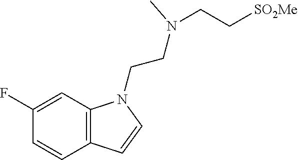 [SMILES: CN(CCn1ccc2ccc(F)cc21)CCS(C)(=O)=O] |
| 13 | 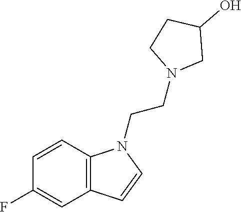 [SMILES: OC1CCN(CCn2ccc3cc(F)ccc32)C1] |
| 14 | 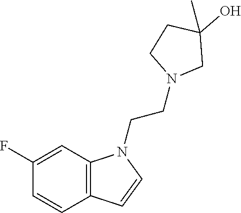 [SMILES: CC1(O)CCN(CCn2ccc3ccc(F)cc32)C1] |
| 15 | 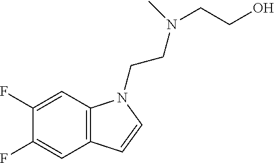 [SMILES: CN(CCO)CCn1ccc2cc(F)c(F)cc21] |
| 16 | 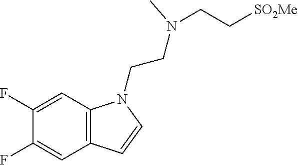 [SMILES: CN(CCn1ccc2cc(F)c(F)cc21)CCS(C)(=O)=O] |
| 17 | 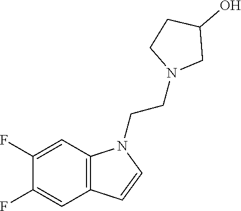 [SMILES: OC1CCN(CCn2ccc3cc(F)c(F)cc32)C1] |
| 18 | 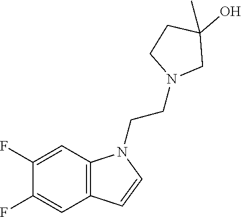 [SMILES: CC1(O)CCN(CCn2ccc3cc(F)c(F)cc32)C1] |
| 19 | 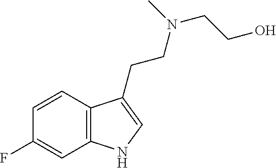 [SMILES: CN(CCO)CCc1c[nH]c2cc(F)ccc12] |
| 20 | 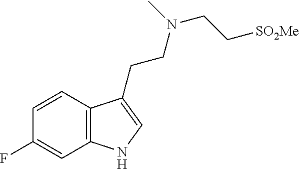 [SMILES: CN(CCc1c[nH]c2cc(F)ccc12)CCS(C)(=O)=O] |
| 21 | 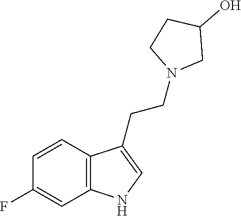 [SMILES: OC1CCN(CCc2c[nH]c3cc(F)ccc23)C1] |
| 22 | 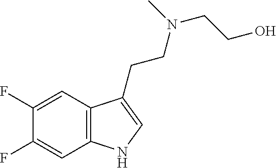 [SMILES: CN(CCO)CCc1c[nH]c2cc(F)c(F)cc12] |
| 23 | 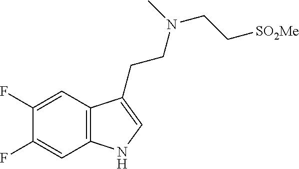 [SMILES: CN(CCc1c[nH]c2cc(F)c(F)cc12)CCS(C)(=O)=O] |
| 24 | 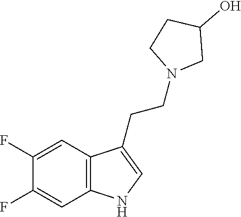 [SMILES: OC1CCN(CCc2c[nH]c3cc(F)c(F)cc23)C1] |
| 25 | 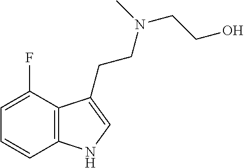 [SMILES: CN(CCO)CCc1c[nH]c2cccc(F)c12] |
| 26 | 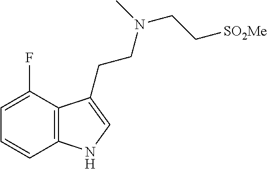 [SMILES: CN(CCc1c[nH]c2cccc(F)c12)CCS(C)(=O)=O] |
| 27 | 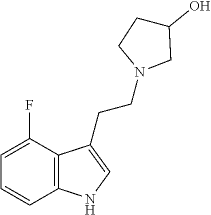 [SMILES: OC1CCN(CCc2c[nH]c3cccc(F)c23)C1] |
| 28 | 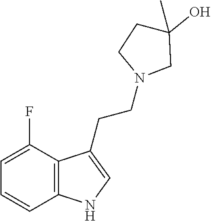 [SMILES: CC1(O)CCN(CCc2c[nH]c3cccc(F)c23)C1] |
| 29 | 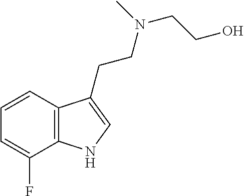 [SMILES: CN(CCO)CCc1c[nH]c2c(F)cccc12] |
| 30 | 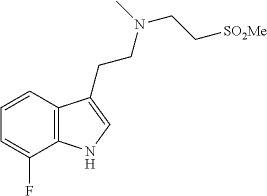 [SMILES: CN(CCc1c[nH]c2c(F)cccc12)CCS(C)(=O)=O] |
| 31 | 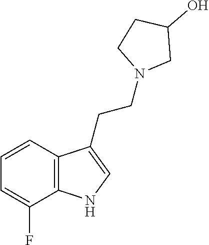 [SMILES: OC1CCN(CCc2c[nH]c3c(F)cccc23)C1] |
| 32 | 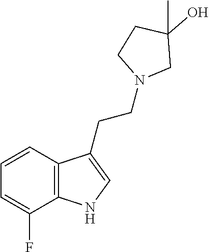 [SMILES: CC1(O)CCN(CCc2c[nH]c3c(F)cccc23)C1] |
| 33 | 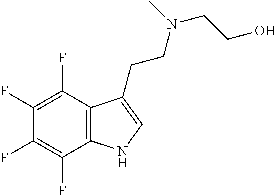 [SMILES: CN(CCO)CCc1c[nH]c2c(F)c(F)c(F)c(F)c12] |
| 34 | 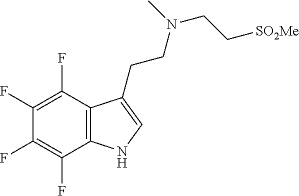 [SMILES: CN(CCc1c[nH]c2c(F)c(F)c(F)c(F)c12)CCS(C)(=O)=O] |
| 35 | 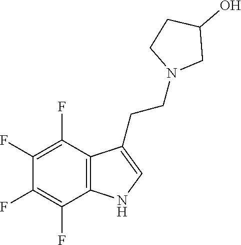 [SMILES: OC1CCN(CCc2c[nH]c3c(F)c(F)c(F)c(F)c23)C1] |
| 36 | 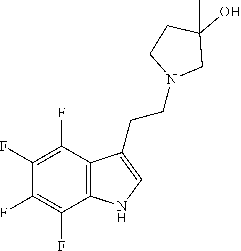 [SMILES: CC1(O)CCN(CCc2c[nH]c3c(F)c(F)c(F)c(F)c23)C1] |
| 37 | 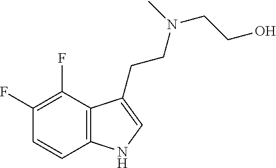 [SMILES: CN(CCO)CCc1c[nH]c2ccc(F)c(F)c12] |
| 38 | 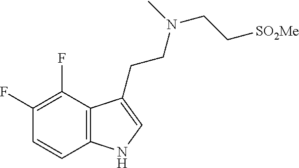 [SMILES: CN(CCc1c[nH]c2ccc(F)c(F)c12)CCS(C)(=O)=O] |
| 39 | 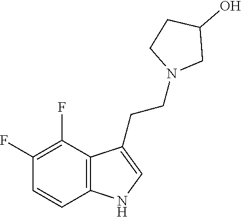 [SMILES: OC1CCN(CCc2c[nH]c3ccc(F)c(F)c23)C1] |
| 40 | 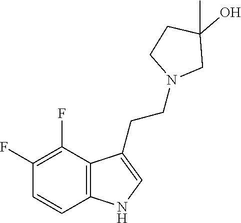 [SMILES: CC1(O)CCN(CCc2c[nH]c3ccc(F)c(F)c23)C1] |
| 41 | 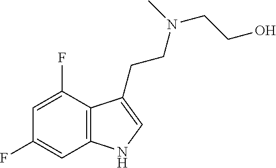 [SMILES: CN(CCO)CCc1c[nH]c2cc(F)cc(F)c12] |
| 42 | 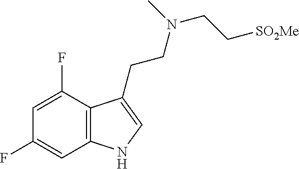 [SMILES: CN(CCc1c[nH]c2cc(F)cc(F)c12)CCS(C)(=O)=O] |
| 43 | 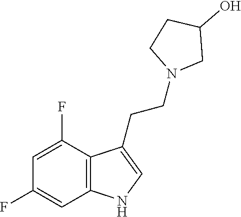 [SMILES: OC1CCN(CCc2c[nH]c3cc(F)cc(F)c23)C1] |
| 44 | 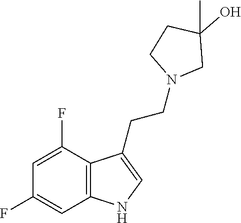 [SMILES: CC1(O)CCN(CCc2c[nH]c3cc(F)cc(F)c23)C1] |
| 45 | 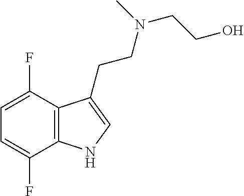 [SMILES: CN(CCO)CCc1c[nH]c2c(F)ccc(F)c12] |
| 46 | 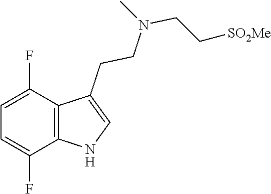 [SMILES: CN(CCc1c[nH]c2c(F)ccc(F)c12)CCS(C)(=O)=O] |
| 47 | 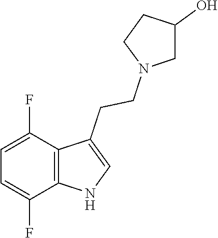 [SMILES: OC1CCN(CCc2c[nH]c3c(F)ccc(F)c23)C1] |
| 48 | 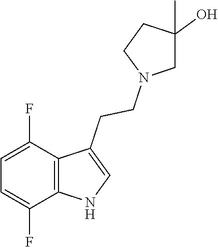 [SMILES: CC1(O)CCN(CCc2c[nH]c3c(F)ccc(F)c23)C1] |
| 49 | 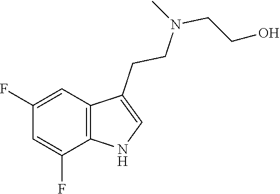 [SMILES: CN(CCO)CCc1c[nH]c2c(F)cc(F)cc12] |
| 50 | 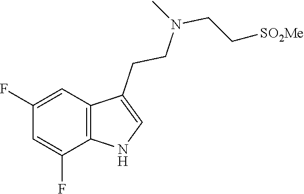 [SMILES: CN(CCc1c[nH]c2c(F)cc(F)cc12)CCS(C)(=O)=O] |
| 51 | 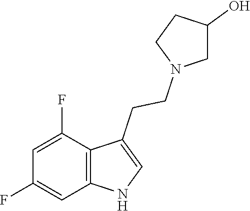 [SMILES: OC1CCN(CCc2c[nH]c3cc(F)cc(F)c23)C1] |
| 52 | 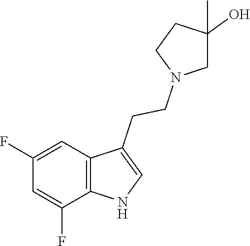 [SMILES: CC1(O)CCN(CCc2c[nH]c3c(F)cc(F)cc23)C1] |
| 53 | 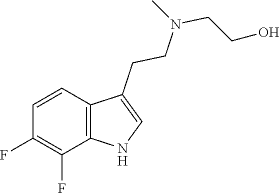 [SMILES: CN(CCO)CCc1c[nH]c2c(F)c(F)ccc12] |
| 54 | 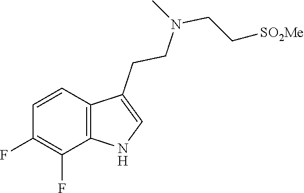 [SMILES: CN(CCc1c[nH]c2c(F)c(F)ccc12)CCS(C)(=O)=O] |
| 55 | 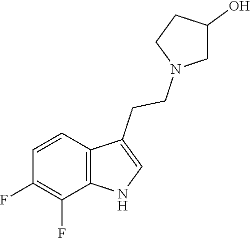 [SMILES: OC1CCN(CCc2c[nH]c3c(F)c(F)ccc23)C1] |
| 56 | 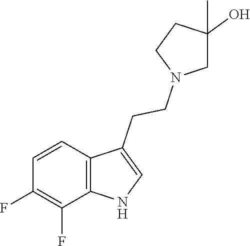 [SMILES: CC1(O)CCN(CCc2c[nH]c3c(F)c(F)ccc23)C1] |
| 57 | 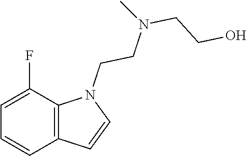 [SMILES: CN(CCO)CCn1ccc2cccc(F)c21] |
| 58 | 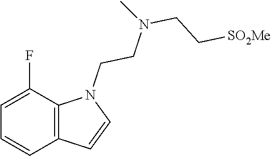 [SMILES: CN(CCn1ccc2cccc(F)c21)CCS(C)(=O)=O] |
| 59 | 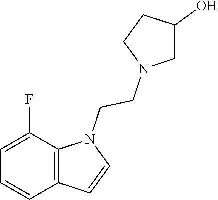 [SMILES: OC1CCN(CCn2ccc3cccc(F)c32)C1] |
| 60 | 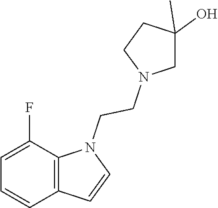 [SMILES: CC1(O)CCN(CCn2ccc3cccc(F)c32)C1] |
| 61 | 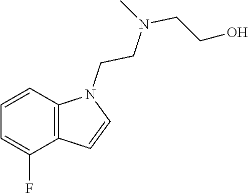 [SMILES: CN(CCO)CCn1ccc2c(F)cccc21] |
| 62 | 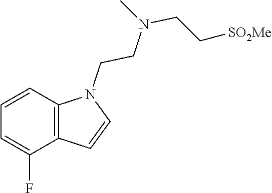 [SMILES: CN(CCn1ccc2c(F)cccc21)CCS(C)(=O)=O] |
| 63 | 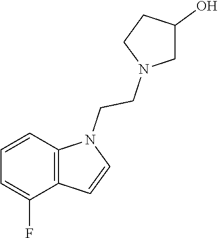 [SMILES: OC1CCN(CCn2ccc3c(F)cccc32)C1] |
| 64 | 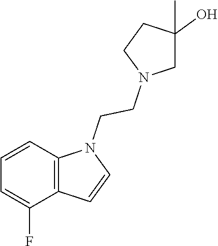 [SMILES: CC1(O)CCN(CCn2ccc3c(F)cccc32)C1] |
| 65 | 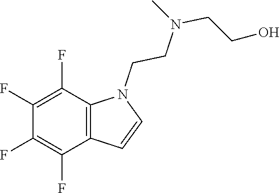 [SMILES: CN(CCO)CCn1ccc2c(F)c(F)c(F)c(F)c21] |
| 66 | 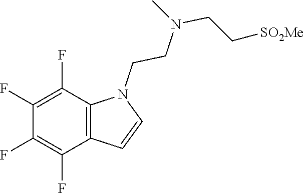 [SMILES: CN(CCn1ccc2c(F)c(F)c(F)c(F)c21)CCS(C)(=O)=O] |
| 67 | 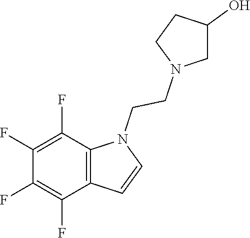 [SMILES: OC1CCN(CCn2ccc3c(F)c(F)c(F)c(F)c32)C1] |
| 68 | 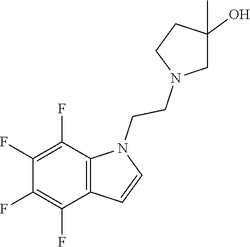 [SMILES: CC1(O)CCN(CCn2ccc3c(F)c(F)c(F)c(F)c32)C1] |
| 69 | 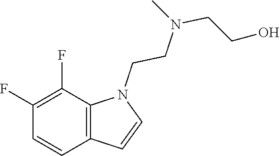 [SMILES: CN(CCO)CCn1ccc2ccc(F)c(F)c21] |
| 70 | 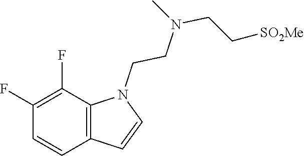 [SMILES: CN(CCn1ccc2ccc(F)c(F)c21)CCS(C)(=O)=O] |
| 71 | 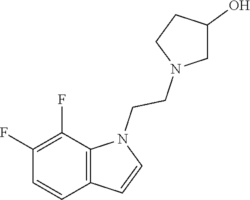 [SMILES: OC1CCN(CCn2ccc3ccc(F)c(F)c32)C1] |
| 72 | 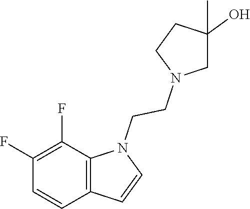 [SMILES: CC1(O)CCN(CCn2ccc3ccc(F)c(F)c32)C1] |
| 73 | 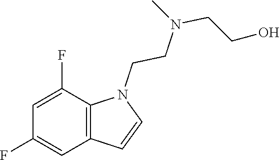 [SMILES: CN(CCO)CCn1ccc2cc(F)cc(F)c21] |
| 74 | 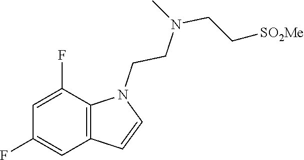 [SMILES: CN(CCn1ccc2cc(F)cc(F)c21)CCS(C)(=O)=O] |
| 75 | 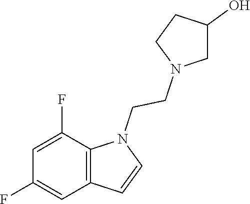 [SMILES: OC1CCN(CCn2ccc3cc(F)cc(F)c32)C1] |
| 76 | 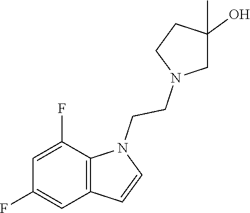 [SMILES: CC1(O)CCN(CCn2ccc3cc(F)cc(F)c32)C1] |
| 77 | 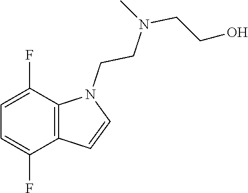 [SMILES: CN(CCO)CCn1ccc2c(F)ccc(F)c21] |
| 78 | 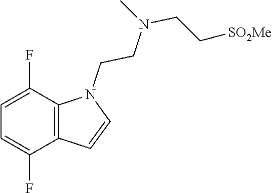 [SMILES: CN(CCn1ccc2c(F)ccc(F)c21)CCS(C)(=O)=O] |
| 79 | 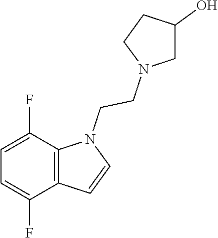 [SMILES: OC1CCN(CCn2ccc3c(F)ccc(F)c32)C1] |
| 80 | 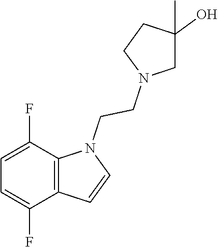 [SMILES: CC1(O)CCN(CCn2ccc3c(F)ccc(F)c32)C1] |
| 81 | 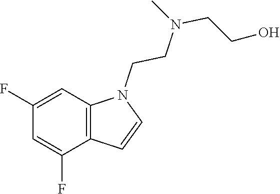 [SMILES: CN(CCO)CCn1ccc2c(F)cc(F)cc21] |
| 82 | 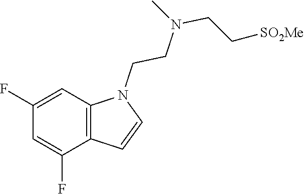 [SMILES: CN(CCn1ccc2c(F)cc(F)cc21)CCS(C)(=O)=O] |
| 83 | 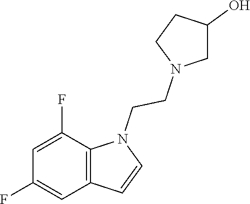 [SMILES: OC1CCN(CCn2ccc3cc(F)cc(F)c32)C1] |
| 84 | 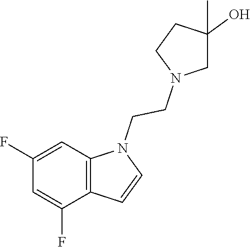 [SMILES: CC1(O)CCN(CCn2ccc3c(F)cc(F)cc32)C1] |
| 85 | 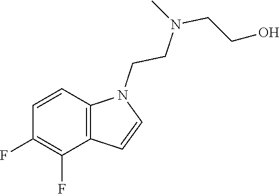 [SMILES: CN(CCO)CCn1ccc2c(F)c(F)ccc21] |
| 86 | 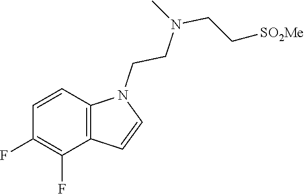 [SMILES: CN(CCn1ccc2c(F)c(F)ccc21)CCS(C)(=O)=O] |
| 87 | 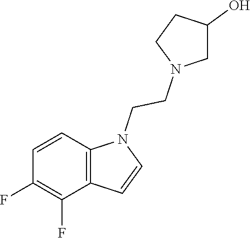 [SMILES: OC1CCN(CCn2ccc3c(F)c(F)ccc32)C1] |
| 88 | 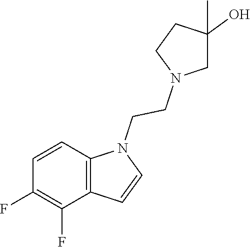 [SMILES: CC1(O)CCN(CCn2ccc3c(F)c(F)ccc32)C1] |
| 89 | 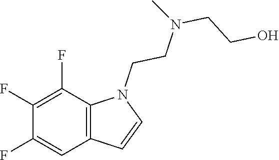 [SMILES: CN(CCO)CCn1ccc2cc(F)c(F)c(F)c21] |
| 90 | 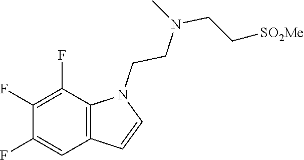 [SMILES: CN(CCn1ccc2cc(F)c(F)c(F)c21)CCS(C)(=O)=O] |
| 91 | 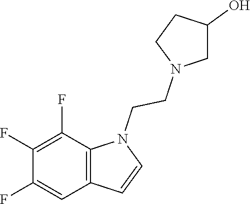 [SMILES: OC1CCN(CCn2ccc3cc(F)c(F)c(F)c32)C1] |
| 92 | 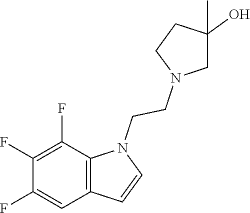 [SMILES: CC1(O)CCN(CCn2ccc3cc(F)c(F)c(F)c32)C1] |
| 93 | 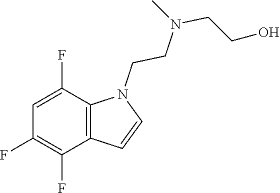 [SMILES: CN(CCO)CCn1ccc2c(F)c(F)cc(F)c21] |
| 94 | 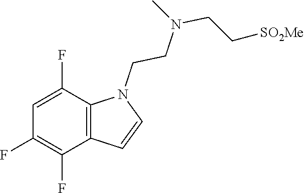 [SMILES: CN(CCn1ccc2c(F)c(F)cc(F)c21)CCS(C)(=O)=O] |
| 95 | 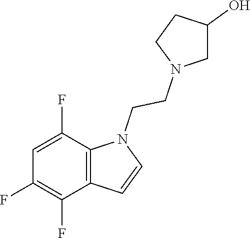 [SMILES: OC1CCN(CCn2ccc3c(F)c(F)cc(F)c32)C1] |
| 96 | 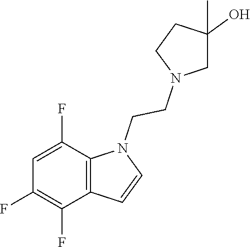 [SMILES: CC1(O)CCN(CCn2ccc3c(F)c(F)cc(F)c32)C1] |
| 97 | 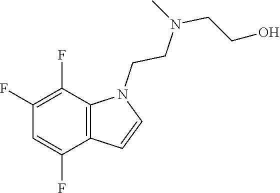 [SMILES: CN(CCO)CCn1ccc2c(F)cc(F)c(F)c21] |
| 98 | 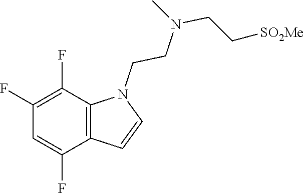 [SMILES: CN(CCn1ccc2c(F)cc(F)c(F)c21)CCS(C)(=O)=O] |
| 99 | 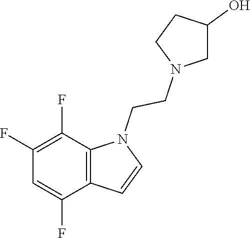 [SMILES: OC1CCN(CCn2ccc3c(F)cc(F)c(F)c32)C1] |
| 100 | 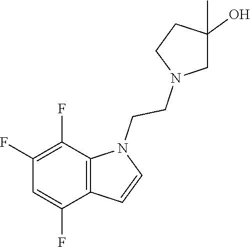 [SMILES: CC1(O)CCN(CCn2ccc3c(F)cc(F)c(F)c32)C1] |
| 101 | 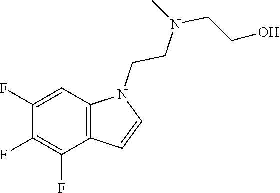 [SMILES: CN(CCO)CCn1ccc2c(F)c(F)c(F)cc21] |
| 102 | 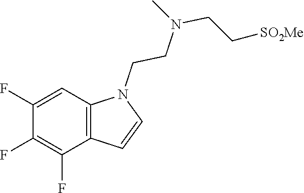 [SMILES: CN(CCn1ccc2c(F)c(F)c(F)cc21)CCS(C)(=O)=O] |
| 103 | 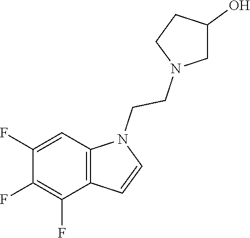 [SMILES: OC1CCN(CCn2ccc3c(F)c(F)c(F)cc32)C1] |
| 104 | 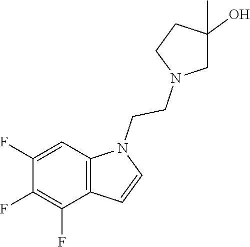 [SMILES: CC1(O)CCN(CCn2ccc3c(F)c(F)c(F)cc32)C1] |
| 105 | 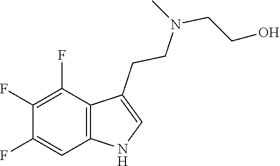 [SMILES: CN(CCO)CCc1c[nH]c2cc(F)c(F)c(F)c12] |
| 106 | 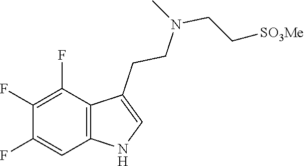 |
| 107 | 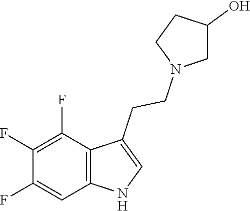 [SMILES: OC1CCN(CCc2c[nH]c3cc(F)c(F)c(F)c23)C1] |
| 108 | 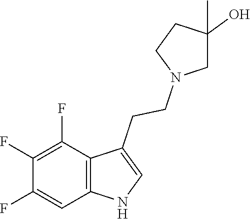 [SMILES: CC1(O)CCN(CCc2c[nH]c3cc(F)c(F)c(F)c23)C1] |
| 109 | 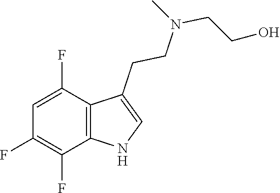 [SMILES: CN(CCO)CCc1c[nH]c2c(F)c(F)cc(F)c12] |
| 110 | 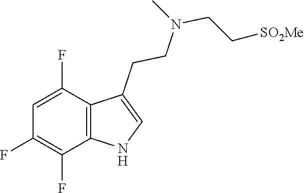 [SMILES: CN(CCc1c[nH]c2c(F)c(F)cc(F)c12)CCS(C)(=O)=O] |
| 111 | 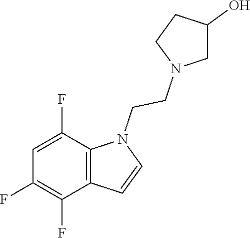 [SMILES: OC1CCN(CCn2ccc3c(F)c(F)cc(F)c32)C1] |
| 112 | 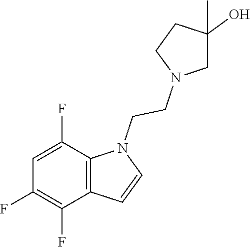 [SMILES: CC1(O)CCN(CCn2ccc3c(F)c(F)cc(F)c32)C1] |
| 113 | 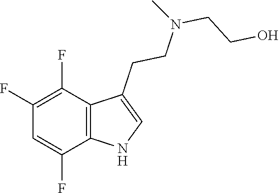 [SMILES: CN(CCO)CCc1c[nH]c2c(F)cc(F)c(F)c12] |
| 114 | 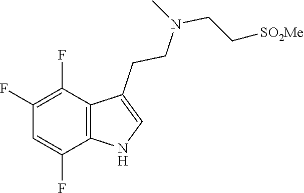 [SMILES: CN(CCc1c[nH]c2c(F)cc(F)c(F)c12)CCS(C)(=O)=O] |
| 115 | 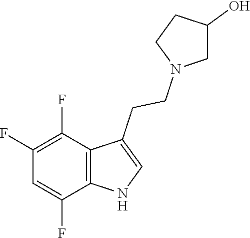 [SMILES: OC1CCN(CCc2c[nH]c3c(F)cc(F)c(F)c23)C1] |
| 116 | 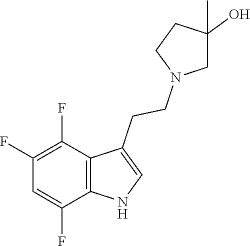 [SMILES: CC1(O)CCN(CCc2c[nH]c3c(F)cc(F)c(F)c23)C1] |
| 117 | 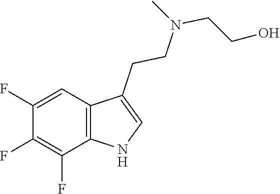 [SMILES: CN(CCO)CCc1c[nH]c2c(F)c(F)c(F)cc12] |
| 118 | 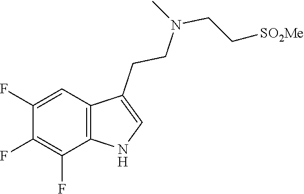 [SMILES: CN(CCc1c[nH]c2c(F)c(F)c(F)cc12)CCS(C)(=O)=O] |
| 119 | 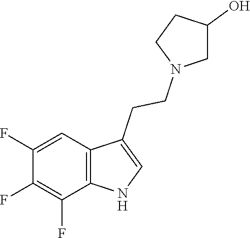 [SMILES: OC1CCN(CCc2c[nH]c3c(F)c(F)c(F)cc23)C1] |
| 120 | 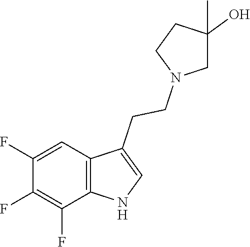 [SMILES: CC1(O)CCN(CCc2c[nH]c3c(F)c(F)c(F)cc23)C1] |

or a pharmaceutically acceptable salt thereof.

### III. Pharmaceutical Compositions and Formulations

In some embodiments, the present invention provides a pharmaceutical composition comprising at least one compound according to the present invention, such as a composition comprising at least compound satisfying any of Table 1 or Formulas (I, II, III, IV, V, I.a, V.a), illustrated above, and a pharmaceutically acceptable excipient. Such compositions are suitable for administration to a subject, such as a human subject.

The presently disclosed pharmaceutical compositions can be prepared in a wide variety of oral, parenteral and topical dosage forms. Oral preparations include tablets, pills, powder, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compounds of the present invention.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

In powders, the carrier is a finely divided solid, which is in a mixture with the finely divided active component, such as at least one compound according to any of Table 1 or Formulas (I, II, III, IV, V, I.a, V.a). In tablets, the active component is mixed with the carrier having the necessary binding properties in suitable proportions and compacted in the shape and size desired. The powders and tablets preferably contain from 5% to 70% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethylcellulose, or sodium carboxymethylcellulose; and gums including arabic and tragacanth; as well as proteins including, but not limited to, gelatin and collagen.

If desired, disintegrating or solubilizing agents may be added, such as cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool and solidify.

Liquid form preparations include solutions, suspensions, and emulsions, for example, water or water/propylene glycol solutions. For parenteral injection, liquid preparations can be formulated in solution, such as in an aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing a finely divided active component, such as at least one compound according to any of Table 1 or Formulas (I, II, III, IV, V, I.a, V.a) in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives, such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to at least one active component according to any of Table 1 or Formulas (I, II, III, IV, V, I.a, V.a), colorants, flavors, stabilizers, buffers, artificial and natural sweeteners, dispersants, thickeners, solubilizing agents, and the like.

Oil suspensions can be formulated by suspending at least one compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J Pharmacol. Exp. Ther. 281:93-102, 1997.

Pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the pharmaceutical compositions of the present invention can be formulated for parenteral administration, such as intravenous (IV) administration or administration into a body cavity or lumen of an organ. The formulations for administration will commonly comprise a solution of a composition according to the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any oil can be employed including synthetic mono- or diglycerides. In addition, fatty acids, such as oleic acid, can likewise be used to prepare injectables. These solutions are preferably sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like.

The concentration of the compositions of the present invention in these formulations can vary, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions of the present invention can be delivered using liposomes, which fuse with the cellular membrane or are endocytosed, for example, by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### IV. Administration

Compounds and compositions of the present invention can be administered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention suitable for a unit dose administration. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound, or compounds, of the present invention can be present in any suitable amount, and can depend on various factors including, but not limited to, weight and age of the subject, state of the disease, and the like as is known to those of ordinary skill in the art. Suitable dosage ranges for the compounds disclosed herein include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds disclosed herein can be administered at any suitable frequency, interval and duration. For example, the compounds can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

Disclosed compositions can include at least one compound according to the present invention, at least two compounds according to the present invention, and/or can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

A compound, or compounds, of the present invention can be co-administered with a second active agent. Co-administration includes administering the compound of the present invention and the second active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, such as by preparing a single pharmaceutical composition including both the compound of the present invention and a second active agent. In other embodiments, the compound of the present invention and the second active agent can be formulated separately.

The disclosed compounds and the second active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the second active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods disclosed herein.

### V. Methods of Treatment

Compounds of the present invention according to any of Table 1 or Formulas (I, II, III, IV, V, I.a, V.a) can be administered for a variety of purposes, including increasing neuronal plasticity, treating any brain disease, and/or increasing at least one of translation, transcription or secretion of neurotrophic factors.

In some embodiments, a compound or composition of the present invention, such as a compound of any of Table 1 or Formulas (I, II, III, IV, V, I.a, V.a), is used to treat a neurological disease. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, the present invention provides a method of treating a disease, including administering to a subject in need thereof, a therapeutically effective amount of at least compound according to any of Table 1 or Formulas (I, II, III, IV, V, I.a, V.a). In some embodiments, the disease is a musculoskeletal pain disorder including fibromyalgia, muscle pain, joint stiffness, osteoarthritis, rheumatoid arthritis, muscle cramps. In some embodiments, the present invention provides a method of treating a disease of women's reproductive health including premenstrual dysphoric disorder (PMDD), premenstrual syndrome (PMS), post-partum depression, and menopause.

In some embodiments, the compounds of the present invention, such as a compound of Table 1 or Formulas (I, II, III, IV, V, I.a, V.a) have activity as 5-HT_{2A} modulators. In some embodiments, the compounds of the present invention elicit a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example, DMT, LSD, and DOI. In some embodiments, the compounds of the present invention are 5-HT_{2A} modulators and promote neural plasticity (e.g., cortical structural plasticity). In some embodiments, the compounds of the present invention are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. The presently disclosed compounds of Table 1 or Formulas (I, II, III, IV, V, I.a, V.a) can function as 5-HT_{2A} agonists alone, or in combination with a second therapeutic agent that also is a 5-HT_{2A} modulator. In such cases the second therapeutic agent can be an agonist or an antagonist. In some instances, it may be helpful to administer a 5-HT_{2A} antagonist in combination with at least one compound of the present invention to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, 3-(2-(4-(4-Fluorobenzoyl)piperazin-1-yl)ethyl)-5-methyl-5-phenylimidazolidine-2,4-dione, 3-(2-(4-Benzhydrylpiperazin-1-yl)ethyl)-5-methyl-5-phenylimidazolidine-2,4-dione, 3-(3-(4-(2-Fluorophenyl)piperazin-1-yl)propyl)-5-methyl-5-phenylimidazolidine-2,4-dione, 3-(3-(4-(3-Fluorophenyl)piperazin-1-yl)propyl)-5-methyl-5-phenylimidazolidine-2,4-dione, 3-(3-(4-(4-Fluorophenyl)piperazin-1-yl)propyl)-5-methyl-5-phenylimidazolidine-2,4-dione, 3-(3-(4-(4-Fluorobenzoyl)piperazin-1-yl)propyl)-5-methyl-5-phenylimidazolidine-2,4-dione, 3-(2-(4-(4-Fluorobenzoyl)piperazin-1-yl)ethyl)-8-phenyl-1,3-diazaspiro[4.5]decane-2,4-dione, 3-(2-(4-Benzhydrylpiperazin-1-yl)ethyl)-8-phenyl-1,3-diazaspiro[4.5]decane-2,4-dione, 3-(3-(4-(2-Fluorophenyl)piperazin-1-yl)propyl)-8-phenyl-1,3-diazaspiro[4.5]decane-2,4-dione, 3-(3-(4-(3-Fluorophenyl)piperazin-1-yl)propyl)-8-phenyl-1,3-diazaspiro[4.5]decane-2,4-dione, 3-(3-(4-(4-Fluorophenyl)piperazin-1-yl)propyl)-8-phenyl-1,3-diazaspiro[4.5]decane-2,4-dione, and 3-(3-(4-(4-Fluorobenzoyl)piperazin-1-yl)propyl)-8-phenyl-1,3-diazaspiro[4.5]decane-2,4-dione, or a pharmaceutically acceptable salt, solvate, metabolite, deuterated analog, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered prior to a compound disclosed herein, such as about three or about hours prior administration of a compound according to Table 1 or Formulas (I, II, III, IV, V, I.a, V.a). In some embodiments, the serotonin receptor modulator is administered at most about one hour prior to the presently disclosed compound. Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present invention is provided at a dose of from about 10 mg to about 100 mg, or from about 20 to about 200 mg, or from about 15 to about 300 mg, and the serotonin receptor modulator is provided at a dose of about 10 mg to about 100 mg.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-FIT_{2A} agonists) are used for increasing at least one of translation, transcription, or secretion of neurotrophic factors.

In some embodiments the presently disclosed compounds of Table 1 or Formulas (I, II, III, IV, V, I.a, V.a) are administered to patients at a dose lower than would produce noticeable psychedelic effects but high enough to provide a therapeutic benefit. This dose range is predicted to be between 200 μg (micrograms) and 2 mg.

### VI. Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with at least one compound of Table 1 or Formulas (I, II, III, IV, V, I.a, V.a) can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present invention provides methods for increasing neuronal plasticity, comprising contacting a neuronal cell with a compound of the present invention, such as a compound of Table 1 or Formulas (I, II, III, IV, V, I.a, V.a). In some embodiments, increasing neuronal plasticity improves a brain disorder.

In some embodiments, a compound of the present invention is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present invention is a mouse head-twitch response (HTR) assay.

In some embodiments, the present invention provides a method for increasing neuronal plasticity, comprising contacting a neuronal cell with a compound of Table 1 or Formulas (I, II, III, IV, V, I.a, V.a).

### VII. Methods of Treating a Brain Disorder

In some embodiments, the present invention provides a method of treating a disease, including administering to a subject in need thereof, a therapeutically effective amount of a compound of the present invention, such as at least one compound of Table 1 or Formulas (I, II, III, IV, V, I.a, V.a). In some embodiments, the disease is a musculoskeletal pain disorder including fibromyalgia, muscle pain, joint stiffness, osteoarthritis, rheumatoid arthritis, muscle cramps. In some embodiments, the present invention provides a method of treating a disease of women's reproductive health including premenstrual dysphoric disorder (PMDD), premenstrual syndrome (PMS), post-partum depression, and menopause. In some embodiments, the present invention provides a method of treating a brain disorder, including administering to a subject in need thereof, a therapeutically effective amount of a compound of the present invention. In some embodiments, the present invention provides a method of treating a brain disorder with combination therapy, including administering to a subject in need thereof, a therapeutically effective amount of at least one compound of the present invention and at least one additional therapeutic agent.

In some embodiments, 5-HT_{2A} modulators (e.g., 5-HT_{2A} agonists) are used to treat a brain disorder. In some embodiments, the brain disorders comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present invention, such as a compound of Table 1 or Formulas (I, II, III, IV, V, I.a, V.a), is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present invention provides a method of treating a brain disorder, comprising administering to a subject in need thereof a therapeutically effective amount of at least one compound disclosed herein, such as a compound of Table 1 or Formulas (I, II, III, IV, V, I.a, V.a).

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agents, such as lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Pamate), diazepam (Valium), alprazolam (Xanax), or clonazepam (Klonopin).

In certain disclosed embodiments, a method for treating a brain disorder disclosed herein comprises administering at least one compound according to Table 1 or Formulas (I, II, III, IV, V, I.a, V.a) and a second empathogenic agent. Examples of suitable empathogenic agents for use in combination with a compound according to Table 1 or Formulas (I, II, III, IV, V, I.a, V.a) are selected from the phenethylamines, such as 3,4-methylenedioxymethamphetamine (MDMA) and analogs thereof. Other suitable empathogenic agents for use in combination with the presently disclosed compounds include, without limitation, N-Allyl-3,4-methylenedioxy-amphetamine (MDAL) N-Butyl-3,4-methylenedioxyamphetamine (MDBU) N-Benzyl-3,4-methylenedioxyamphetamine (MDBZ) N-Cyclopropylmethyl-3,4-methylenedioxy amphetamine (MDCPM) N,N-Dimethyl-3,4-methylenedioxyamphetamine (MDDM) N-Ethyl-3,4-methylenedioxyamphetamine (MDE; MDEA) N-(2-Hydroxyethyl)-3,4-methylenedioxy amphetamine (MDHOET) N-Isopropyl-3,4-methylenedioxyamphetamine (MDIP) N-Methyl-3,4-ethylenedioxyamphetamine (MDMC) N-Methoxy-3,4-methylenedioxyamphetamine (MDMEO) N-(2-Methoxyethyl)-3,4-methylenedioxyamphetamine (MDMEOET) alpha,alpha,N-Trimethyl-3,4-methylenedioxyphenethylamine (MDMP; 3,4-Methylenedioxy-N-methylphentermine) N-Hydroxy-3,4-methylenedioxyamphetamine (MDOH) 3,4-Methylenedioxyphenethylamine (MDPEA) alpha,alpha-Dimethyl-3,4-methylenedioxyphenethylamine (MDPH; 3,4-methylenedioxyphentermine) N-Propargyl-3,4-methylenedioxyamphetamine (MDPL) Methylenedioxy-2-aminoindane (MDAI) N-methyl-1,3-benzodioxolylbutanamine (MBDB) 3,4-methylenedioxy-N-methyl-α-ethylphenylethylamine 3,4-Methylenedioxyamphetamine MDA Methylone (also known as “3,4-methylenedioxy-N-methylcathinone) Ethylone, also known as 3,4-methylenedioxy-N-ethylcathinone GHB or Gamma Hydroxybutyrate or sodium oxybate N-Propyl-3,4-methylenedioxyamphetamine (MDPR), and the like.

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### VIII. Methods of Increasing at Least One of Translation, Transcription, or Secretion of Neurotrophic Factors

Neurotrophic factors refer to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increase neuronal plasticity. In some embodiments, increasing at least one of translation, transcription, or secretion of neurotrophic factors can promote neuronal growth, promote neuritogenesis, promote synaptogenesis, promote dendritogenesis, increase dendritic arbor complexity, and/or increase dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present invention, such as a compound of Table 1 or Formulas (I, II, III, IV, V, I.a, V.a), is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present invention provides a method for increasing at least one of translation, transcription or secretion of neurotrophic factors, comprising contacting a neuronal cell with a compound disclosed herein, such as at least one compound of Table 1 or Formulas (I, II, III, IV, V, I.a, V.a).

### IX. Combination Therapy

In particular embodiments of treating the disorders described above, combination therapy is used as described herein. In such therapy a compound disclosed herein, including those described in Table 1, is administered in combination with a serotonin receptor modulator. In certain embodiments the serotonin receptor modulator is selected from the group consisting of altanserin, blonanserin, eplivanserin, glemanserin, volinanserin, ketanserin, ritanserin, pimavanserin, nelotanserin, pruvanserin, and flibanserin. In one embodiment, the serotonin receptor modulator is selected from the group consisting of serotonin receptor modulator is selected from the group consisting of eplivanserin, volinanserin, ketanserin, ritanserin, pimavanserin, nelotanserin, pruvanserin, flibanserin, olanzapine, quetiapine, risperidone, and buspirone.

In some embodiments, the serotonin receptor modulator for use with the compounds disclosed herein, including those described in Table 1, is eplivanserin, wherein the eplivanserin is administered in about 1 mg to about 40 mg, or about 5 mg to about 10 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is volinanserin, wherein the volinanserin is administered in about 1 mg to about 60 mg, or about 5 mg to about 20 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is ketanserin, wherein the ketanserin is administered in about 10 mg to about 80 mg, about 30 mg to about 50 mg, or about 40 mg and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is ritanserin, wherein the ritanserin is administered in about 1 mg to about 40 mg, or about 2.5 mg to about 10 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is pimavanserin, wherein the pimavanserin is administered in about 1 mg to about 60 mg, or about 17 mg to about 34 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is nelotanserin, wherein the nelotanserin is administered in about 1 mg to about 80 mg, or about 40 mg to about 80 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is pruvanserin, wherein the pruvanserin is administered in about 1 mg to about 40 mg, or about 3 mg to about 10 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is flibanserin, wherein the flibanserin is administered in about 10 mg to about 200 mg, or about 80 mg to about 120 mg, or about 100 mg and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is olanzapine, wherein the olanzapine is administered in about 2.5 mg to about 30 mg, or about 5 mg or about 10 mg, or about 20 mg or about 25 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is an extended-release of olanzapine such as ZYPREXA RELPREVV, wherein the extended release olanzapine is administered in about 50 mg to about 450 mg, or about 150 mg or about 210 mg, or about 300 mg or about 405 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is quetiapine, wherein the quetiapine is administered in about 25 mg to about 800 mg, or about 50 mg to about 100 mg, or about 150 mg or about 200 mg or about 250 mg or about 300 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is an extended-release of quetiapine, wherein the extended-release of quetiapine is administered in about 50 mg to about 300 mg, or about 50 mg or about 100 mg or about 200 mg, or about 300 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is risperidone, wherein the risperidone is administered in about 0.5 mg to about 20 mg or about 0.5 mg, or about 1 mg, or about 2 mg, or about 3 mg or about 4 mg or about 5 mg or about 7.5 mg or about 10 mg or about 16 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is an extended-release of risperidone including (RISPERDAL CONSTA), wherein the extended-release of risperidone is administered in about 12.5 mg, or about 25 mg, or about 37.5 mg, or about 50 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In some embodiments, the serotonin receptor modulator for use with the compounds disclosed herein, including those described in Table 1, is buspirone, wherein the buspirone is administered in about 1 mg to about 100 mg, or about 1 mg or about 2 mg, or about 3 mg, or about 4 mg, or about 5 mg, or about 6 mg, or about 7 mg, or about 7.5 mg, or about 10 mg, or about 15 mg, or about 22.5 mg, or about 30 mg, or about 37.5 mg, or about 45 mg, or about 52.5 mg, or about 60 mg, or about 1 mg to about 10 mg, or about 5 mg to about 10 mg, or about 10 mg to about 15 mg, or about 15 mg to about 30 mg, or about 30 mg to about 60 mg, or about 60 mg to about 80 mg, or about 80 mg to about 100 mg, and the compounds disclosed herein, including those described in Table 1, are administered between about 1 mg to about 100 mg, or about 1 mg to about 60 mg, or about 1 mg to about 30 mg, or about 0.1 mg/kg to about 0.3 mg/kg, or about 0.1 mg/kg followed by 0.3 mg/kg, or about 0.1 mg/kg, or about 0.3 mg/kg.

In certain embodiments, such as those described above, a compound disclosed herein, including those described in Table 1, is co-administered with a serotonin receptor modulator in the same or in separate compositions. In one embodiment, the compound disclosed herein, including those described in Table 1, is administered in a modified release formulation such that the subject is effectively pretreated with serotonin receptor modulator prior to release of an effective amount of the compound disclosed herein, including those described in Table 1. Thus, in some embodiments, the serotonin receptor modulator is administered or released from a composition provided herein prior to the administration and/or release of the psychedelic. This allows pretreatment to attenuate activation of the serotonin receptor by the psychedelic.

In some embodiments, the serotonin receptor modulator is administered or released from the composition provided herein to pretreat a subject by at least about at about 5 minutes, 10 minutes, 20 minutes, 30 minutes, 40 minutes, 50 minutes, 1 hour, 1.25 hours, 1.5 hours, 2 hours, or 3 hours prior to the release of the psychedelic. In some embodiments, the serotonin receptor modulator attenuates the activation of the serotonin receptor when the serotonin receptor modulator is used to pretreat at most about 3 hours, 4 hours, 5 hours, 6 hours, 7 hours, 8 hours, 9 hours, or more than 9 hours prior to the release of the psychedelic. In some embodiments, the serotonin receptor modulator attenuates the activation of the serotonin receptor when the serotonin receptor modulator is used to pretreat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour prior to the release of the psychedelic.

In a preferred embodiment, the serotonin receptor modulator is administered at about 1 hour to about 3 hours prior to the administration of the psychedelic.

In some embodiments, the serotonin receptor modulator is eplivanserin and the psychedelic is a compound disclosed herein, including those described in Table 1, wherein the eplivanserin is administered to pretreat at least 15 minutes prior to the administration of the compound disclosed herein, including those described in Table 1.

In some embodiments, the serotonin receptor modulator is eplivanserin and the psychedelic is a compound disclosed herein, including those described in Table 1, wherein the eplivanserin is administered to pretreat between at least 30 minutes prior and 360 minutes prior to the release or administration of the compound disclosed herein, including those described in Table 1. In some embodiments, the serotonin receptor modulator is eplivanserin and the psychedelic is a compound disclosed herein, including those described in Table 1, wherein the eplivanserin is administered to pretreat between at least 60 minutes prior and 360 minutes prior to the release or administration of the compound disclosed herein, including those described in Table 1. In some embodiments, the serotonin receptor modulator is eplivanserin and the psychedelic is a compound disclosed herein, including those described in Table 1, wherein the eplivanserin is administered to pretreat between at least 90 minutes and 240 minutes prior to the compound disclosed herein, including those described in Table 1.

In some embodiments, the serotonin receptor modulator is eplivanserin and the psychedelic is a compound disclosed herein, including those described in Table 1, wherein the eplivanserin is administered to pretreat at least 120 minutes prior to the compound disclosed herein, including those described in Table 1.

In some embodiments, the serotonin receptor modulator is eplivanserin and the psychedelic is a compound disclosed herein, including those described in Table 1, wherein the eplivanserin is administered to pretreat at least 150 minutes prior to the compound disclosed herein, including those described in Table 1. In some embodiments, the serotonin receptor modulator is eplivanserin and the psychedelic is a compound disclosed herein, including those described in Table 1, wherein the eplivanserin is administered to pretreat between about 15 minutes and about 150 minutes prior to the compound disclosed herein, including those described in Table 1. In some embodiments, the serotonin receptor modulator is eplivanserin and the psychedelic is a compound disclosed herein, including those described in Table 1, wherein the eplivanserin is administered to pretreat at least 180 minutes prior to the compound disclosed herein, including those described in Table 1. In some embodiments, the serotonin receptor modulator is eplivanserin and the psychedelic is a compound disclosed herein, including those described in Table 1, wherein the eplivanserin is administered to pretreat at least 210 minutes prior to the compound disclosed herein, including those described in Table 1. In some embodiments, the serotonin receptor modulator is eplivanserin and the psychedelic is a compound disclosed herein, including those described in Table 1, wherein the eplivanserin is administered to pretreat at least 240 minutes prior to the compound disclosed herein, including those described in Table 1.

In some embodiments, the serotonin receptor modulator is eplivanserin and the psychedelic is a compound disclosed herein, including those described in Table 1, wherein the eplivanserin is administered to pretreat at least 270 minutes prior to the compound disclosed herein, including those described in Table 1. In some embodiments, the serotonin receptor modulator is eplivanserin and the psychedelic is a compound disclosed herein, including those described in Table 1, wherein the eplivanserin is administered to pretreat at least 300 minutes prior to the compound disclosed herein, including those described in Table 1. In some embodiments, the serotonin receptor modulator is eplivanserin and the psychedelic is a compound disclosed herein, including those described in Table 1, wherein the eplivanserin is administered to pretreat at least 330 minutes prior to the compound disclosed herein, including those described in Table 1. In some embodiments, the serotonin receptor modulator is eplivanserin and the psychedelic is a compound disclosed herein, including those described in Table 1, wherein the eplivanserin is administered to pretreat at least 360 minutes prior to the compound disclosed herein, including those described in Table 1.

In some preferred embodiments, the serotonin receptor modulator is eplivanserin and the psychedelic is a compound disclosed herein, including those described in Table 1, wherein eplivanserin is administered to pretreat between about 60 minutes and about 180 minutes prior to the administration of the compound disclosed herein, including those described in Table 1.

In some embodiments, the serotonin receptor modulator is volinanserin and the psychedelic is a compound disclosed herein, including those described in Table 1, wherein the volinanserin is administered to pretreat a subject between at least 15 minutes and 360 minutes prior to the administration or release of the compound disclosed herein, including those described in Table 1. In some embodiments, the serotonin receptor modulator is volinanserin and the psychedelic is a compound disclosed herein, including those described in Table 1, wherein the volinanserin is administered to pretreat between at least 30 minutes and 360 minutes prior to the administration or release of the compound disclosed herein, including those described in Table 1. In some embodiments, the serotonin receptor modulator is volinanserin and the psychedelic is a compound disclosed herein, including those described in Table 1, wherein the volinanserin is administered to pretreat between at least 60 minutes and 240 minutes prior to the administration or release of the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 90 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 120 minutes prior to the compound disclosed herein, including those described in Table 1.

In some embodiments, the serotonin receptor modulator is volinanserin and the psychedelic is a compound disclosed herein, including those described in Table 1, wherein the volinanserin is administered to pretreat at least 150 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat between about 15 minutes and about 150 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 180 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 210 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 240 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 270 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 300 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 330 minutes prior to the compound disclosed herein, including those described in Table 1. In some embodiments, the serotonin receptor modulator is volinanserin and the psychedelic is a compound disclosed herein, including those described in Table 1, wherein the volinanserin is administered to pretreat at least 360 minutes prior to the compound disclosed herein, including those described in Table 1. In some preferred embodiments, the serotonin receptor modulator is volinanserin and the psychedelic is a compound disclosed herein, including those described in Table 1, wherein volinanserin is administered to pretreat between about 60 minutes and about 180 minutes prior to the administration of the compound disclosed herein, including those described in Table 1.

In some embodiments, the serotonin receptor modulator is ketanserin and the psychedelic is a compound disclosed herein, including those described in Table 1, wherein the ketanserin is administered to pretreat at least 15 minutes prior to the administration of the compound disclosed herein, including those described in Table 1. In some embodiments, the serotonin receptor modulator is ketanserin and the psychedelic is a compound disclosed herein, including those described in Table 1, wherein the ketanserin is administered to pretreat between at least 30 minutes and 360 minutes prior to the administration or release of the compound disclosed herein, including those described in Table 1. In some embodiments, the serotonin receptor modulator is ketanserin and the psychedelic is a compound disclosed herein, including those described in Table 1, wherein the ketanserin is administered to pretreat between at least 60 minutes and 240 minutes prior to the administration or release of the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 90 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 120 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 150 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat between about 15 minutes and about 150 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 180 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 210 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 240 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 270 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 300 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 330 minutes prior to the compound disclosed herein, including those described in Table 1. In some embodiments, the serotonin receptor modulator is ketanserin and the psychedelic is a compound disclosed herein, including those described in Table 1, wherein the ketanserin is administered to pretreat at least 360 minutes prior to the compound disclosed herein, including those described in Table 1. In some preferred embodiments, the serotonin receptor modulator is ketanserin and the psychedelic is a compound disclosed herein, including those described in Table 1, wherein ketanserin is administered to pretreat between about 60 minutes and about 180 minutes prior to the administration of the compound disclosed herein, including those described in Table 1.

In some embodiments, the serotonin receptor modulator is ritanserin and the psychedelic is a compound disclosed herein, including those described in Table 1, wherein the ritanserin is administered to pretreat at least 15 minutes prior to the administration of the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 30 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat between at least 60 minutes and 240 minutes prior to the administration or release of the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 90 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 120 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 150 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat between about 15 minutes and about 150 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 180 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 210 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 240 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 270 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 300 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 330 minutes prior to the compound disclosed herein, including those described in Table 1. In some embodiments, the serotonin receptor modulator is ritanserin and the psychedelic is a compound disclosed herein, including those described in Table 1, wherein the ritanserin is administered to pretreat at least 360 minutes prior to the compound disclosed herein, including those described in Table 1. In some preferred embodiments, the serotonin receptor modulator is ritanserin and the psychedelic is a compound disclosed herein, including those described in Table 1, wherein ritanserin is administered to pretreat between about 60 minutes and about 180 minutes prior to the administration of the compound disclosed herein, including those described in Table 1.

In some embodiments, the serotonin receptor modulator is pimavanserin and the psychedelic is a compound disclosed herein, including those described in Table 1, wherein the pimavanserin is administered to pretreat at least 15 minutes prior to the administration of the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 30 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat between at least 60 minutes and 240 minutes prior to the administration or release of the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 90 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 120 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 150 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat between about 15 minutes and about 150 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 180 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 210 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 240 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 270 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 300 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 330 minutes prior to the compound disclosed herein, including those described in Table 1. In some embodiments, the serotonin receptor modulator is pimavanserin and the psychedelic is a compound disclosed herein, including those described in Table 1, wherein the pimavanserin is administered to pretreat at least 360 minutes prior to the compound disclosed herein, including those described in Table 1. In some preferred embodiments, the serotonin receptor modulator is pimavanserin and the psychedelic is a compound disclosed herein, including those described in Table 1, wherein pimavanserin is administered to pretreat between about 60 minutes and about 180 minutes prior to the administration of the compound disclosed herein, including those described in Table 1.

In some embodiments, the serotonin receptor modulator is nelotanserin and the psychedelic is a compound disclosed herein, including those described in Table 1, wherein the nelotanserin is administered to pretreat at least 15 minutes prior to the administration of the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 30 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat between at least 60 minutes and 240 minutes prior to the administration or release of the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 90 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 120 minutes prior to the compound disclosed herein, including those described in Table 1.

In some embodiments, the serotonin receptor modulator is nelotanserin and the psychedelic is a compound disclosed herein, including those described in Table 1, wherein the nelotanserin is administered to pretreat at least 150 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat between about 15 minutes and about 150 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 180 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 210 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 240 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 270 minutes prior to the compound disclosed herein, including those described in Table 1.

In some embodiments, the serotonin receptor modulator is nelotanserin and the psychedelic is a compound disclosed herein, including those described in Table 1, wherein the nelotanserin is administered to pretreat at least 300 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 330 minutes prior to the compound disclosed herein, including those described in Table 1. In some embodiments, the serotonin receptor modulator is nelotanserin and the psychedelic is a compound disclosed herein, including those described in Table 1, wherein the nelotanserin is administered to pretreat at least 360 minutes prior to the compound disclosed herein, including those described in Table 1. In some preferred embodiments, the serotonin receptor modulator is nelotanserin and the psychedelic is a compound disclosed herein, including those described in Table 1, wherein nelotanserin is administered to pretreat between about 60 minutes and about 180 minutes prior to the administration of the compound disclosed herein, including those described in Table 1.

In some embodiments, the serotonin receptor modulator is pruvanserin and the psychedelic is a compound disclosed herein, including those described in Table 1, wherein the pruvanserin is administered to pretreat at least 15 minutes prior to the administration of the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 30 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat between at least 60 minutes and 240 minutes prior to the administration or release of the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 90 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 120 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 150 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat between about 15 minutes and about 150 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 180 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 210 minutes prior to the compound disclosed herein, including those described in Table 1.

In some embodiments, the serotonin receptor modulator is pruvanserin and the psychedelic is a compound disclosed herein, including those described in Table 1, wherein the pruvanserin is administered to pretreat at least 240 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 270 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 300 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 330 minutes prior to the compound disclosed herein, including those described in Table 1. In some embodiments, the serotonin receptor modulator is pruvanserin and the psychedelic is a compound disclosed herein, including those described in Table 1, wherein the pruvanserin is administered to pretreat at least 360 minutes prior to the compound disclosed herein, including those described in Table 1. In some preferred embodiments, the serotonin receptor modulator is pruvanserin and the psychedelic is a compound disclosed herein, including those described in Table 1, wherein pruvanserin is administered to pretreat between about 60 minutes and about 180 minutes prior to the administration of the compound disclosed herein, including those described in Table 1.

In some embodiments, the serotonin receptor modulator is flibanserin and the psychedelic is a compound disclosed herein, including those described in Table 1, wherein the flibanserin is administered to pretreat at least 15 minutes prior to the administration of the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 30 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat between at least 60 minutes and 240 minutes prior to the administration or release of the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 90 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 120 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 150 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat between about 15 minutes and about 150 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 180 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 210 minutes prior to the compound disclosed herein, including those described in Table 1.

In some embodiments, the serotonin receptor modulator is flibanserin and the psychedelic is a compound disclosed herein, including those described in Table 1, wherein the flibanserin is administered to pretreat at least 240 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 270 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 300 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 330 minutes prior to the compound disclosed herein, including those described in Table 1. In some embodiments, the serotonin receptor modulator is flibanserin and the psychedelic is a compound disclosed herein, including those described in Table 1, wherein the flibanserin is administered to pretreat at least 360 minutes prior to the compound disclosed herein, including those described in Table 1. In some preferred embodiments, the serotonin receptor modulator is flibanserin and the psychedelic is a compound disclosed herein, including those described in Table 1, wherein flibanserin is administered to pretreat between about 60 minutes and about 180 minutes prior to the administration of the compound disclosed herein, including those described in Table 1.

In some embodiments, the serotonin receptor modulator is olanzapine and the psychedelic is a compound disclosed herein, including those described in Table 1, wherein the olanzapine is administered to pretreat at least 15 minutes prior to the administration of the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 30 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat between at least 60 minutes and 240 minutes prior to the administration or release of the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 90 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 120 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 150 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat between about 15 minutes and about 150 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 180 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 210 minutes prior to the compound disclosed herein, including those described in Table 1.

In some embodiments, the serotonin receptor modulator is olanzapine and the psychedelic is a compound disclosed herein, including those described in Table 1, wherein the olanzapine is administered to pretreat at least 240 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 270 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 300 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 330 minutes prior to the compound disclosed herein, including those described in Table 1. In some embodiments, the serotonin receptor modulator is olanzapine and the psychedelic is a compound disclosed herein, including those described in Table 1, wherein the olanzapine is administered to pretreat at least 360 minutes prior to the compound disclosed herein, including those described in Table 1. In some preferred embodiments, the serotonin receptor modulator is olanzapine and the psychedelic is a compound disclosed herein, including those described in Table 1, wherein olanzapine is administered to pretreat between about 60 minutes and about 180 minutes prior to the administration of the compound disclosed herein, including those described in Table 1.

In some embodiments, the serotonin receptor modulator is risperidone and the psychedelic is a compound disclosed herein, including those described in Table 1, wherein the risperidone is administered to pretreat at least 15 minutes prior to the administration of the compound disclosed herein, including those described in Table 1. In some embodiments, the serotonin receptor modulator is risperidone and the psychedelic is a compound disclosed herein, including those described in Table 1, wherein the risperidone is administered to pretreat at least 30 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat between at least 60 minutes and 240 minutes prior to the administration or release of the compound disclosed herein, including those described in Table 1. In some embodiments, the serotonin receptor modulator is risperidone and the psychedelic is a compound disclosed herein, including those described in Table 1, wherein the risperidone is administered to pretreat at least 90 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat at least 120 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat at least 150 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat between about 15 minutes and about 150 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat at least 180 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat at least 210 minutes prior to the compound disclosed herein, including those described in Table 1.

In some embodiments, the serotonin receptor modulator is risperidone and the psychedelic is a compound disclosed herein, including those described in Table 1, wherein the risperidone is administered to pretreat at least 240 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat at least 270 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat at least 300 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat at least 330 minutes prior to the compound disclosed herein, including those described in Table 1. In some embodiments, the serotonin receptor modulator is risperidone and the psychedelic is a compound disclosed herein, including those described in Table 1, wherein the risperidone is administered to pretreat at least 360 minutes prior to the compound disclosed herein, including those described in Table 1. In some preferred embodiments, the serotonin receptor modulator is risperidone and the psychedelic is a compound disclosed herein, including those described in Table 1, wherein risperidone is administered to pretreat between about 60 minutes and about 180 minutes prior to the administration of the compound disclosed herein, including those described in Table 1.

In some embodiments, the serotonin receptor modulator is quetiapine and the psychedelic is a compound disclosed herein, including those described in Table 1, wherein the quetiapine is administered to pretreat at least 15 minutes prior to the administration of the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 30 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat between at least 60 minutes and 240 minutes prior to the administration or release of the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 90 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 120 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 150 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat between about 15 minutes and about 150 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 180 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 210 minutes prior to the compound disclosed herein, including those described in Table 1.

In some embodiments, the serotonin receptor modulator is quetiapine and the psychedelic is a compound disclosed herein, including those described in Table 1, wherein the quetiapine is administered to pretreat at least 240 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 270 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 300 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 330 minutes prior to the compound disclosed herein, including those described in Table 1. In some embodiments, the serotonin receptor modulator is quetiapine and the psychedelic is a compound disclosed herein, including those described in Table 1, wherein the quetiapine is administered to pretreat at least 360 minutes prior to the compound disclosed herein, including those described in Table 1. In some preferred embodiments, the serotonin receptor modulator is quetiapine and the psychedelic is a compound disclosed herein, including those described in Table 1, wherein quetiapine is administered to pretreat between about 60 minutes and about 180 minutes prior to the administration of the compound disclosed herein, including those described in Table 1.

In some embodiments, the serotonin receptor modulator is buspirone and the psychedelic is a compound disclosed herein, including those described in Table 1, wherein the buspirone is administered to pretreat at least 15 minutes prior to the administration of the compound disclosed herein, including those described in Table 1. In some embodiments, the serotonin receptor modulator is buspirone and the psychedelic is a compound disclosed herein, including those described in Table 1, wherein the buspirone is administered to pretreat at least 30 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat between at least 60 minutes and 240 minutes prior to the administration or release of the compound disclosed herein, including those described in Table 1. In some embodiments, the serotonin receptor modulator is buspirone and the psychedelic is a compound disclosed herein, including those described in Table 1, wherein the buspirone is administered to pretreat at least 90 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat at least 120 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat at least 150 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat between about 15 minutes and about 150 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat at least 180 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat at least 210 minutes prior to the compound disclosed herein, including those described in Table 1.

In some embodiments, the serotonin receptor modulator is buspirone and the psychedelic is a compound disclosed herein, including those described in Table 1, wherein the buspirone is administered to pretreat at least 240 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat at least 270 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat at least 300 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat at least 330 minutes prior to the compound disclosed herein, including those described in Table 1. In some embodiments, the serotonin receptor modulator is buspirone and the psychedelic is a compound disclosed herein, including those described in Table 1, wherein the buspirone is administered to pretreat at least 360 minutes prior to the compound disclosed herein, including those described in Table 1. In some preferred embodiments, the serotonin receptor modulator is buspirone and the psychedelic is a compound disclosed herein, including those described in Table 1, wherein buspirone is administered to pretreat between about 60 minutes and about 180 minutes prior to the administration of the compound disclosed herein, including those described in Table 1.

In certain embodiments, such as those described above, a compound disclosed herein, including those described in Table 1, is co-administered with a serotonin receptor modulator in the same or in separate compositions. In one embodiment, the serotonin receptor modulator is administered after the compound disclosed herein, including those described in Table 1. In one embodiment, the compound disclosed herein, including those described in Table 1, is administered in a modified release formulation such that the subject is effectively post-treated with serotonin receptor modulator post to release of an effective amount of the compound disclosed herein, including those described in Table 1. In some embodiments, the serotonin receptor modulator is part of a single fixed dose formulation that releases the compound disclosed herein, including those described in Table 1, first followed by serotonin receptor modulator on two different release profiles. In another embodiment, the compound disclosed herein, including those described in Table 1, is administered first as a single dosage and, after a length of time, serotonin receptor modulator is administered as a second dosage separate from the first dosage. Thus, in some embodiments, the serotonin receptor modulator is administered or released from a composition provided herein after the administration and/or release of the psychedelic. This allows post-treatment to attenuate activation of the serotonin receptor by the psychedelic.

In some embodiments, the serotonin receptor modulator is administered or released from the composition provided herein to post-treat a subject by at least about at about 5 minutes, 10 minutes, 20 minutes, 30 minutes, 40 minutes, 50 minutes, 1 hour, 1.25 hours, 1.5 hours, 2 hours, or 3 hours after the release of the psychedelic. In some embodiments, the serotonin receptor modulator attenuates the activation of the serotonin receptor when the serotonin receptor modulator is used to post-treat at most about 3 hours, 4 hours, 5 hours, 6 hours, 7 hours, 8 hours, 9 hours, or more than 9 hours after the release of the psychedelic. In some embodiments, the serotonin receptor modulator attenuates the activation of the serotonin receptor when the serotonin receptor modulator is used to post-treat in a range of about 5 minutes to about 3 hours, about 10 minutes to about 3 hours, about 20 minutes to about 3 hours, about 30 minutes to about 3 hours, about 40 minutes to about 3 hours, about 50 minutes to about 3 hours, about 1 hour to about 3 hours, about 5 minutes to about 2 hours, about 10 minutes to about 2 hours, about 20 minutes to about 2 hours, about 30 minutes to about 2 hours, about 40 minutes to about 2 hours, about 50 minutes to about 2 hours, about 1 hour to about 2 hours, about 5 minutes to about 1 hour, about 10 minutes to about 1 hour, about 20 minutes to about 1 hour, about 30 minutes to about 1 hour, about 40 minutes to about 1 hour, or about 50 minutes to about 1 hour after the release of the psychedelic.

In a preferred embodiment, the serotonin receptor modulator is administered at about 1 hour to about 3 hours after the administration of the psychedelic.

In some embodiments, the serotonin receptor modulator is eplivanserin and the psychedelic is a compound disclosed herein, including those described in Table 1, wherein the eplivanserin is administered to post-treat at least 15 minutes after the administration of the compound disclosed herein, including those described in Table 1. In some embodiments, the serotonin receptor modulator is eplivanserin and the psychedelic is a compound disclosed herein, including those described in Table 1, wherein the eplivanserin is administered to post-treat between at least 30 minutes after and 360 minutes after the release or administration of the compound disclosed herein, including those described in Table 1. In some embodiments, the serotonin receptor modulator is eplivanserin and the psychedelic is a compound disclosed herein, including those described in Table 1, wherein the eplivanserin is administered to post-treat between at least 60 minutes after and 360 minutes after the release or administration of the compound disclosed herein, including those described in Table 1. In some embodiments, the serotonin receptor modulator is eplivanserin and the psychedelic is a compound disclosed herein, including those described in Table 1, wherein the eplivanserin is administered to post-treat between at least 90 minutes and 240 minutes after the compound disclosed herein, including those described in Table 1. In some embodiments, the serotonin receptor modulator is eplivanserin and the psychedelic is a compound disclosed herein, including those described in Table 1, wherein the eplivanserin is administered to post-treat at least 120 minutes after the compound disclosed herein, including those described in Table 1.

In some embodiments, the serotonin receptor modulator is eplivanserin and the psychedelic is a compound disclosed herein, including those described in Table 1, wherein the eplivanserin is administered to post-treat at least 150 minutes after the compound disclosed herein, including those described in Table 1. In some embodiments, the serotonin receptor modulator is eplivanserin and the psychedelic is a compound disclosed herein, including those described in Table 1, wherein the eplivanserin is administered to post-treat between about 15 minutes and about 150 minutes after the compound disclosed herein, including those described in Table 1. In some embodiments, the serotonin receptor modulator is eplivanserin and the psychedelic is a compound disclosed herein, including those described in Table 1, wherein the eplivanserin is administered to post-treat at least 180 minutes after the compound disclosed herein, including those described in Table 1. In some embodiments, the serotonin receptor modulator is eplivanserin and the psychedelic is a compound disclosed herein, including those described in Table 1, wherein the eplivanserin is administered to post-treat at least 210 minutes after the compound disclosed herein, including those described in Table 1. In some embodiments, the serotonin receptor modulator is eplivanserin and the psychedelic is a compound disclosed herein, including those described in Table 1, wherein the eplivanserin is administered to post-treat at least 240 minutes after the compound disclosed herein, including those described in Table 1.

In some embodiments, the serotonin receptor modulator is eplivanserin and the psychedelic is a compound disclosed herein, including those described in Table 1, wherein the eplivanserin is administered to post-treat at least 270 minutes after the compound disclosed herein, including those described in Table 1. In some embodiments, the serotonin receptor modulator is eplivanserin and the psychedelic is a compound disclosed herein, including those described in Table 1, wherein the eplivanserin is administered to post-treat at least 300 minutes after the compound disclosed herein, including those described in Table 1. In some embodiments, the serotonin receptor modulator is eplivanserin and the psychedelic is a compound disclosed herein, including those described in Table 1, wherein the eplivanserin is administered to post-treat at least 330 minutes after the compound disclosed herein, including those described in Table 1. In some embodiments, the serotonin receptor modulator is eplivanserin and the psychedelic is a compound disclosed herein, including those described in Table 1, wherein the eplivanserin is administered to post-treat at least 360 minutes after the compound disclosed herein, including those described in Table 1.

In some preferred embodiments, the serotonin receptor modulator is eplivanserin and the psychedelic is a compound disclosed herein, including those described in Table 1, wherein eplivanserin is administered to post-treat between about 60 minutes and about 180 minutes after the administration of the compound disclosed herein, including those described in Table 1.

In some embodiments, the serotonin receptor modulator is volinanserin and the psychedelic is a compound disclosed herein, including those described in Table 1, wherein the volinanserin is administered to post-treat a subject between at least 15 minutes and 360 minutes after the administration or release of the compound disclosed herein, including those described in Table 1. In some embodiments, the serotonin receptor modulator is volinanserin and the psychedelic is a compound disclosed herein, including those described in Table 1, wherein the volinanserin is administered to post-treat between at least 30 minutes and 360 minutes after the administration or release of the compound disclosed herein, including those described in Table 1. In some embodiments, the serotonin receptor modulator is volinanserin and the psychedelic is a compound disclosed herein, including those described in Table 1, wherein the volinanserin is administered to post-treat between at least 60 minutes and 240 minutes after the administration or release of the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 90 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 120 minutes after the compound disclosed herein, including those described in Table 1.

In some embodiments, the serotonin receptor modulator is volinanserin and the psychedelic is a compound disclosed herein, including those described in Table 1, wherein the volinanserin is administered to post-treat at least 150 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat between about 15 minutes and about 150 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 180 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 210 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 240 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 270 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 300 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 330 minutes after the compound disclosed herein, including those described in Table 1. In some embodiments, the serotonin receptor modulator is volinanserin and the psychedelic is a compound disclosed herein, including those described in Table 1, wherein the volinanserin is administered to post-treat at least 360 minutes after the compound disclosed herein, including those described in Table 1. In some preferred embodiments, the serotonin receptor modulator is volinanserin and the psychedelic is a compound disclosed herein, including those described in Table 1, wherein volinanserin is administered to post-treat between about 60 minutes and about 180 minutes after the administration of the compound disclosed herein, including those described in Table 1.

In some embodiments, the serotonin receptor modulator is ketanserin and the psychedelic is a compound disclosed herein, including those described in Table 1, wherein the ketanserin is administered to post-treat at least 15 minutes after the administration of the compound disclosed herein, including those described in Table 1. In some embodiments, the serotonin receptor modulator is ketanserin and the psychedelic is a compound disclosed herein, including those described in Table 1, wherein the ketanserin is administered to post-treat between at least 30 minutes and 360 minutes after the administration or release of the compound disclosed herein, including those described in Table 1. In some embodiments, the serotonin receptor modulator is ketanserin and the psychedelic is a compound disclosed herein, including those described in Table 1, wherein the ketanserin is administered to post-treat between at least 60 minutes and 240 minutes after the administration or release of the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 90 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 120 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 150 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat between about 15 minutes and about 150 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 180 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 210 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 240 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 270 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 300 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 330 minutes after the compound disclosed herein, including those described in Table 1. In some embodiments, the serotonin receptor modulator is ketanserin and the psychedelic is a compound disclosed herein, including those described in Table 1, wherein the ketanserin is administered to post-treat at least 360 minutes after the compound disclosed herein, including those described in Table 1. In some preferred embodiments, the serotonin receptor modulator is ketanserin and the psychedelic is a compound disclosed herein, including those described in Table 1, wherein ketanserin is administered to post-treat between about 60 minutes and about 180 minutes after the administration of the compound disclosed herein, including those described in Table 1.

In some embodiments, the serotonin receptor modulator is ritanserin and the psychedelic is a compound disclosed herein, including those described in Table 1, wherein the ritanserin is administered to post-treat at least 15 minutes after the administration of the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 30 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat between at least 60 minutes and 240 minutes after the administration or release of the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 90 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 120 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 150 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat between about 15 minutes and about 150 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 180 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 210 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 240 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 270 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 300 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 330 minutes after the compound disclosed herein, including those described in Table 1. In some embodiments, the serotonin receptor modulator is ritanserin and the psychedelic is a compound disclosed herein, including those described in Table 1, wherein the ritanserin is administered to post-treat at least 360 minutes after the compound disclosed herein, including those described in Table 1. In some preferred embodiments, the serotonin receptor modulator is ritanserin and the psychedelic is a compound disclosed herein, including those described in Table 1, wherein ritanserin is administered to post-treat between about 60 minutes and about 180 minutes after the administration of the compound disclosed herein, including those described in Table 1.

In some embodiments, the serotonin receptor modulator is pimavanserin and the psychedelic is a compound disclosed herein, including those described in Table 1, wherein the pimavanserin is administered to post-treat at least 15 minutes after the administration of the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 30 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat between at least 60 minutes and 240 minutes after the administration or release of the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 90 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 120 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 150 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat between about 15 minutes and about 150 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 180 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 210 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 240 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 270 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 300 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 330 minutes after the compound disclosed herein, including those described in Table 1. In some embodiments, the serotonin receptor modulator is pimavanserin and the psychedelic is a compound disclosed herein, including those described in Table 1, wherein the pimavanserin is administered to post-treat at least 360 minutes after the compound disclosed herein, including those described in Table 1. In some preferred embodiments, the serotonin receptor modulator is pimavanserin and the psychedelic is a compound disclosed herein, including those described in Table 1, wherein pimavanserin is administered to post-treat between about 60 minutes and about 180 minutes after the administration of the compound disclosed herein, including those described in Table 1.

In some embodiments, the serotonin receptor modulator is nelotanserin and the psychedelic is a compound disclosed herein, including those described in Table 1, wherein the nelotanserin is administered to post-treat at least 15 minutes after the administration of the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 30 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat between at least 60 minutes and 240 minutes after the administration or release of the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 90 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 120 minutes after the compound disclosed herein, including those described in Table 1.

In some embodiments, the serotonin receptor modulator is nelotanserin and the psychedelic is a compound disclosed herein, including those described in Table 1, wherein the nelotanserin is administered to post-treat at least 150 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat between about 15 minutes and about 150 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 180 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 210 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 240 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 270 minutes after the compound disclosed herein, including those described in Table 1.

In some embodiments, the serotonin receptor modulator is nelotanserin and the psychedelic is a compound disclosed herein, including those described in Table 1, wherein the nelotanserin is administered to post-treat at least 300 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 330 minutes after the compound disclosed herein, including those described in Table 1. In some embodiments, the serotonin receptor modulator is nelotanserin and the psychedelic is a compound disclosed herein, including those described in Table 1, wherein the nelotanserin is administered to post-treat at least 360 minutes after the compound disclosed herein, including those described in Table 1. In some preferred embodiments, the serotonin receptor modulator is nelotanserin and the psychedelic is a compound disclosed herein, including those described in Table 1, wherein nelotanserin is administered to post-treat between about 60 minutes and about 180 minutes after the administration of the compound disclosed herein, including those described in Table 1.

In some embodiments, the serotonin receptor modulator is pruvanserin and the psychedelic is a compound disclosed herein, including those described in Table 1, wherein the pruvanserin is administered to post-treat at least 15 minutes after the administration of the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 30 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat between at least 60 minutes and 240 minutes after the administration or release of the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 90 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 120 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 150 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat between about 15 minutes and about 150 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 180 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 210 minutes after the compound disclosed herein, including those described in Table 1.

In some embodiments, the serotonin receptor modulator is pruvanserin and the psychedelic is a compound disclosed herein, including those described in Table 1, wherein the pruvanserin is administered to post-treat at least 240 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 270 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 300 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 330 minutes after the compound disclosed herein, including those described in Table 1. In some embodiments, the serotonin receptor modulator is pruvanserin and the psychedelic is a compound disclosed herein, including those described in Table 1, wherein the pruvanserin is administered to post-treat at least 360 minutes after the compound disclosed herein, including those described in Table 1. In some preferred embodiments, the serotonin receptor modulator is pruvanserin and the psychedelic is a compound disclosed herein, including those described in Table 1, wherein pruvanserin is administered to post-treat between about 60 minutes and about 180 minutes after the administration of the compound disclosed herein, including those described in Table 1.

In some embodiments, the serotonin receptor modulator is flibanserin, wherein the flibanserin is administered to post-treat at least 15 minutes post to the administration of the compound disclosed herein, including those described in Table 1. In some embodiments, the serotonin receptor modulator is flibanserin, wherein the flibanserin is administered to post-treat at least 30 minutes post to the compound disclosed herein, including those described in Table 1. In some embodiments, the serotonin receptor modulator is flibanserin, wherein the flibanserin is administered to post-treat between at least 60 minutes and 240 minutes post to the administration or release of the compound disclosed herein, including those described in Table 1. In some embodiments, the serotonin receptor modulator is flibanserin, wherein the flibanserin is administered to post-treat at least 90 minutes post to the compound disclosed herein, including those described in Table 1. In some embodiments, the serotonin receptor modulator is flibanserin, wherein the flibanserin is administered to post-treat at least 120 minutes post to the compound disclosed herein, including those described in Table 1. In some embodiments, the serotonin receptor modulator is flibanserin, wherein the flibanserin is administered to post-treat at least 150 minutes post to the compound disclosed herein, including those described in Table 1. In some embodiments, the serotonin receptor modulator is flibanserin, wherein the flibanserin is administered to post-treat between about 15 minutes and about 150 minutes post to the compound disclosed herein, including those described in Table 1. In some embodiments, the serotonin receptor modulator is flibanserin, wherein the flibanserin is administered to post-treat at least 180 minutes post to the compound disclosed herein, including those described in Table 1. In some embodiments, the serotonin receptor modulator is flibanserin, wherein the flibanserin is administered to post-treat at least 210 minutes post to the compound disclosed herein, including those described in Table 1.

In some embodiments, the serotonin receptor modulator is flibanserin, wherein the flibanserin is administered to post-treat at least 240 minutes post to the compound disclosed herein, including those described in Table 1. In some embodiments, the serotonin receptor modulator is flibanserin, wherein the flibanserin is administered to post-treat at least 270 minutes post to the compound disclosed herein, including those described in Table 1. In some embodiments, the serotonin receptor modulator is flibanserin, wherein the flibanserin is administered to post-treat at least 300 minutes post to the compound disclosed herein, including those described in Table 1. In some embodiments, the serotonin receptor modulator is flibanserin, wherein the flibanserin is administered to post-treat at least 330 minutes post to the compound disclosed herein, including those described in Table 1. In some embodiments, the serotonin receptor modulator is flibanserin, wherein the flibanserin is administered to post-treat at least 360 minutes post to the compound disclosed herein, including those described in Table 1. In some preferred embodiments, the serotonin receptor modulator is flibanserin, wherein flibanserin is administered to post-treat between about 60 minutes and about 180 minutes post to the administration of the compound disclosed herein, including those described in Table 1.

In some embodiments, the serotonin receptor modulator is olanzapine, wherein the olanzapine is administered to post-treat at least 15 minutes post to the administration of the compound disclosed herein, including those described in Table 1. In some embodiments, the serotonin receptor modulator is olanzapine, wherein the olanzapine is administered to post-treat at least 30 minutes post to the compound disclosed herein, including those described in Table 1. In some embodiments, the serotonin receptor modulator is olanzapine, wherein the olanzapine is administered to post-treat between at least 60 minutes and 240 minutes post to the administration or release of the compound disclosed herein, including those described in Table 1. In some embodiments, the serotonin receptor modulator is olanzapine, wherein the olanzapine is administered to post-treat at least 90 minutes post to the compound disclosed herein, including those described in Table 1. In some embodiments, the serotonin receptor modulator is olanzapine, wherein the olanzapine is administered to post-treat at least 120 minutes post to the compound disclosed herein, including those described in Table 1. In some embodiments, the serotonin receptor modulator is olanzapine, wherein the olanzapine is administered to post-treat at least 150 minutes post to the compound disclosed herein, including those described in Table 1. In some embodiments, the serotonin receptor modulator is olanzapine, wherein the olanzapine is administered to post-treat between about 15 minutes and about 150 minutes post to the compound disclosed herein, including those described in Table 1. In some embodiments, the serotonin receptor modulator is olanzapine, wherein the olanzapine is administered to post-treat at least 180 minutes post to the compound disclosed herein, including those described in Table 1. In some embodiments, the serotonin receptor modulator is olanzapine, wherein the olanzapine is administered to post-treat at least 210 minutes post to the compound disclosed herein, including those described in Table 1.

In some embodiments, the serotonin receptor modulator is olanzapine, wherein the olanzapine is administered to post-treat at least 240 minutes post to the compound disclosed herein, including those described in Table 1. In some embodiments, the serotonin receptor modulator is olanzapine, wherein the olanzapine is administered to post-treat at least 270 minutes post to the compound disclosed herein, including those described in Table 1. In some embodiments, the serotonin receptor modulator is olanzapine, wherein the olanzapine is administered to post-treat at least 300 minutes post to the compound disclosed herein, including those described in Table 1. In some embodiments, the serotonin receptor modulator is olanzapine, wherein the olanzapine is administered to post-treat at least 330 minutes post to the compound disclosed herein, including those described in Table 1. In some embodiments, the serotonin receptor modulator is olanzapine, wherein the olanzapine is administered to post-treat at least 360 minutes post to the compound disclosed herein, including those described in Table 1. In some preferred embodiments, the serotonin receptor modulator is olanzapine, wherein olanzapine is administered to post-treat between about 60 minutes and about 180 minutes post to the administration of the compound disclosed herein, including those described in Table 1.

In some embodiments, the serotonin receptor modulator is quetiapine, wherein the quetiapine is administered to post-treat at least 15 minutes post to the administration of the compound disclosed herein, including those described in Table 1. In some embodiments, the serotonin receptor modulator is quetiapine, wherein the quetiapine is administered to post-treat at least 30 minutes post to the compound disclosed herein, including those described in Table 1. In some embodiments, the serotonin receptor modulator is quetiapine, wherein the quetiapine is administered to post-treat between at least 60 minutes and 240 minutes post to the administration or release of the compound disclosed herein, including those described in Table 1. In some embodiments, the serotonin receptor modulator is quetiapine, wherein the quetiapine is administered to post-treat at least 90 minutes post to the compound disclosed herein, including those described in Table 1. In some embodiments, the serotonin receptor modulator is quetiapine, wherein the quetiapine is administered to post-treat at least 120 minutes post to the compound disclosed herein, including those described in Table 1. In some embodiments, the serotonin receptor modulator is quetiapine, wherein the quetiapine is administered to post-treat at least 150 minutes post to the compound disclosed herein, including those described in Table 1. In some embodiments, the serotonin receptor modulator is quetiapine, wherein the quetiapine is administered to post-treat between about 15 minutes and about 150 minutes post to the compound disclosed herein, including those described in Table 1. In some embodiments, the serotonin receptor modulator is quetiapine, wherein the quetiapine is administered to post-treat at least 180 minutes post to the compound disclosed herein, including those described in Table 1. In some embodiments, the serotonin receptor modulator is quetiapine, wherein the quetiapine is administered to post-treat at least 210 minutes post to the compound disclosed herein, including those described in Table 1.

In some embodiments, the serotonin receptor modulator is quetiapine, wherein the quetiapine is administered to post-treat at least 240 minutes post to the compound disclosed herein, including those described in Table 1. In some embodiments, the serotonin receptor modulator is quetiapine, wherein the quetiapine is administered to post-treat at least 270 minutes post to the compound disclosed herein, including those described in Table 1. In some embodiments, the serotonin receptor modulator is quetiapine, wherein the quetiapine is administered to post-treat at least 300 minutes post to the compound disclosed herein, including those described in Table 1. In some embodiments, the serotonin receptor modulator is quetiapine, wherein the quetiapine is administered to post-treat at least 330 minutes post to the compound disclosed herein, including those described in Table 1. In some embodiments, the serotonin receptor modulator is quetiapine, wherein the quetiapine is administered to post-treat at least 360 minutes post to the compound disclosed herein, including those described in Table 1. In some preferred embodiments, the serotonin receptor modulator is quetiapine, wherein quetiapine is administered to post-treat between about 60 minutes and about 180 minutes post to the administration of the compound disclosed herein, including those described in Table 1.

In some embodiments, the serotonin receptor modulator is risperidone, wherein the risperidone is administered to post-treat at least 15 minutes post to the administration of the compound disclosed herein, including those described in Table 1. In some embodiments, the serotonin receptor modulator is risperidone, wherein the risperidone is administered to post-treat at least 30 minutes post to the compound disclosed herein, including those described in Table 1. In some embodiments, the serotonin receptor modulator is risperidone, wherein the risperidone is administered to post-treat between at least 60 minutes and 240 minutes post to the administration or release of the compound disclosed herein, including those described in Table 1. In some embodiments, the serotonin receptor modulator is risperidone, wherein the risperidone is administered to post-treat at least 90 minutes post to the compound disclosed herein, including those described in Table 1. In some embodiments, the serotonin receptor modulator is risperidone, wherein the risperidone is administered to post-treat at least 120 minutes post to the compound disclosed herein, including those described in Table 1. In some embodiments, the serotonin receptor modulator is risperidone, wherein the risperidone is administered to post-treat at least 150 minutes post to the compound disclosed herein, including those described in Table 1. In some embodiments, the serotonin receptor modulator is risperidone, wherein the risperidone is administered to post-treat between about 15 minutes and about 150 minutes post to the compound disclosed herein, including those described in Table 1. In some embodiments, the serotonin receptor modulator is risperidone, wherein the risperidone is administered to post-treat at least 180 minutes post to the compound disclosed herein, including those described in Table 1. In some embodiments, the serotonin receptor modulator is risperidone, wherein the risperidone is administered to post-treat at least 210 minutes post to the compound disclosed herein, including those described in Table 1.

In some embodiments, the serotonin receptor modulator is risperidone, wherein the risperidone is administered to post-treat at least 240 minutes post to the compound disclosed herein, including those described in Table 1. In some embodiments, the serotonin receptor modulator is risperidone, wherein the risperidone is administered to post-treat at least 270 minutes post to the compound disclosed herein, including those described in Table 1. In some embodiments, the serotonin receptor modulator is risperidone, wherein the risperidone is administered to post-treat at least 300 minutes post to the compound disclosed herein, including those described in Table 1. In some embodiments, the serotonin receptor modulator is risperidone, wherein the risperidone is administered to post-treat at least 330 minutes post to the compound disclosed herein, including those described in Table 1. In some embodiments, the serotonin receptor modulator is risperidone, wherein the risperidone is administered to post-treat at least 360 minutes post to the compound disclosed herein, including those described in Table 1. In some preferred embodiments, the serotonin receptor modulator is risperidone, wherein risperidone is administered to post-treat between about 60 minutes and about 180 minutes post to the administration of the compound disclosed herein, including those described in Table 1.

In some embodiments, the serotonin receptor modulator is buspirone, wherein the buspirone is administered to post-treat at least 15 minutes post to the administration of the compound disclosed herein, including those described in Table 1. In some embodiments, the serotonin receptor modulator is buspirone, wherein the buspirone is administered to post-treat at least 30 minutes post to the compound disclosed herein, including those described in Table 1. In some embodiments, the serotonin receptor modulator is buspirone, wherein the buspirone is administered to post-treat between at least 60 minutes and 240 minutes post to the administration or release of the compound disclosed herein, including those described in Table 1. In some embodiments, the serotonin receptor modulator is buspirone, wherein the buspirone is administered to post-treat at least 90 minutes post to the compound disclosed herein, including those described in Table 1. In some embodiments, the serotonin receptor modulator is buspirone, wherein the buspirone is administered to post-treat at least 120 minutes post to the compound disclosed herein, including those described in Table 1. In some embodiments, the serotonin receptor modulator is buspirone, wherein the buspirone is administered to post-treat at least 150 minutes post to the compound disclosed herein, including those described in Table 1. In some embodiments, the serotonin receptor modulator is buspirone, wherein the buspirone is administered to post-treat between about 15 minutes and about 150 minutes post to the compound disclosed herein, including those described in Table 1. In some embodiments, the serotonin receptor modulator is buspirone, wherein the buspirone is administered to post-treat at least 180 minutes post to the compound disclosed herein, including those described in Table 1. In some embodiments, the serotonin receptor modulator is buspirone, wherein the buspirone is administered to post-treat at least 210 minutes post to the compound disclosed herein, including those described in Table 1.

In some embodiments, the serotonin receptor modulator is buspirone, wherein the buspirone is administered to post-treat at least 240 minutes post to the compound disclosed herein, including those described in Table 1. In some embodiments, the serotonin receptor modulator is buspirone, wherein the buspirone is administered to post-treat at least 270 minutes post to the compound disclosed herein, including those described in Table 1. In some embodiments, the serotonin receptor modulator is buspirone, wherein the buspirone is administered to post-treat at least 300 minutes post to the compound disclosed herein, including those described in Table 1. In some embodiments, the serotonin receptor modulator is buspirone, wherein the buspirone is administered to post-treat at least 330 minutes post to the compound disclosed herein, including those described in Table 1. In some embodiments, the serotonin receptor modulator is buspirone, wherein the buspirone is administered to post-treat at least 360 minutes post to the compound disclosed herein, including those described in Table 1. In some preferred embodiments, the serotonin receptor modulator is buspirone, wherein buspirone is administered to post-treat between about 60 minutes and about 180 minutes post to the administration of the compound disclosed herein, including those described in Table 1.

### X. Agonist and Antagonist Profiles at Select 5-hydroxytryptamine (5-HT; Serotonin) Receptors

Protocol: in vitro testing at select 5-hydroxytryptamine (5-HT; serotonin) receptors was conducted.

### Assay Design: Calcium Mobilization

### Cell Handling

1. Cell lines were expanded from freezer stocks according to standard procedures.
2. Cells were seeded in a total volume of 20 μL into black-walled, clear-bottom, Poly-D-lysine coated 384-well microplates and incubated at 37° C. for the appropriate time prior to testing.

### Dye Loading

1. Assays were performed in 1×Dye Loading Buffer consisting of 1× Dye, 1× Additive A and 2.5 mM Probenecid in HBSS/20 mM Hepes. Probenicid was prepared fresh.
2. Cells were loaded with dye prior to testing. Media was aspirated from cells and replaced with 20 μL Dye Loading Buffer.
3. Cells were incubated for 30-60 minutes at 37° C.

### Agonist Format

1. For agonist determination, cells were incubated with sample to induce response.
2. After dye loading, cells were removed from the incubator and 10 μL HBSS/20 mM Hepes was added. 3× vehicle was included in the buffer when performing agonist dose curves to define the EC80 for subsequent antagonist assays. Cells were incubated for 30 minutes at room temperature in the dark to equilibrate plate temperature.
3. Intermediate dilution of sample stocks was performed to generate 4× sample in assay buffer.
4. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

### Allosteric Modulation Format

1. For allosteric determination, cells were pre-incubated with sample followed by agonist induction at the EC20 concentration.
2. Intermediate dilution of sample stocks was performed to generate 3× sample in assay buffer.
3. After dye loading, cells were removed from the incubator and 10 μL 3× sample was added. Cells were incubated for 30 minutes at room temperature in the dark to equilibrate plate temperature. Vehicle concentration was 1%.
4. Compound allosteric activity was measured on a FLIPR Tetra (MDS). Calcium mobilization was monitored for 2 minutes and 10 μL of 4× EC20 agonist in HBSS/20 mM Hepes was added to the cells 5 seconds into the assay.

### Antagonist Format

1. For antagonist determination, cells were pre-incubated with sample followed by agonist challenge at the EC80 concentration.
2. Intermediate dilution of sample stocks was performed to generate 3× sample in assay buffer.
3. After dye loading, cells were removed from the incubator and 10 μL 3× sample was added. Cells were incubated for 30 minutes at room temperature in the dark to equilibrate plate temperature. Vehicle concentration was 1%.
4. Compound antagonist activity was measured on a FLIPR Tetra (MDS). Calcium mobilization was monitored for 2 minutes and 10 μL EC80 agonist in HBSS/20 mM Hepes was added to the cells 5 seconds into the assay.

### Data Analysis

1. Compound activity was analyzed using CBIS data analysis suite (ChemInnovation, CA).
2. For agonist mode assays, percentage activity is calculated using the following formula:

% Activity=100%×(mean RFU of test sample−mean RFU of vehicle control)/(mean MAX RFU control ligand −mean RFU of vehicle control).
3. For positive allosteric mode assays, percentage modulation was calculated using the following formula:

% Modulation=100%×((mean RFU of test sample−mean RFU of EC20 control)/(mean RFU of MAX control ligand −mean RFU of EC20 control)).
4. For antagonist and negative allosteric modulation mode assays, percentage inhibition is calculated using the following formula:

% Inhibition=100%×(1−(mean RFU of test sample−mean RFU of vehicle control)/(mean RFU of EC80 control−mean RFU of vehicle control)).

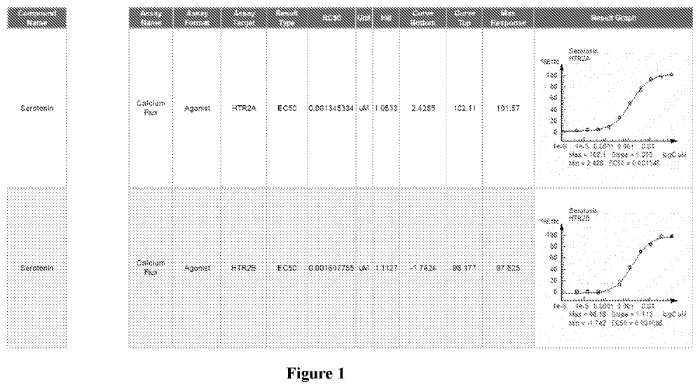

FIG. 1 shows control dose response curves for the selected GPCR Biosensor Assays.

TABLE 2
Activity of compounds against 5-HT2A and 5-HT2B receptors.

| Structure & Name | 5-HT2A agonist efficacy @ 1 μM | 5-HT2B agonist efficacy @ 1 μM |
|---|---|---|
| 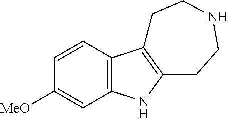 [SMILES: COc1ccc2c3c([nH]c2c1)CCNCC3] 8-methoxy-1,2,3,4,5,6- hexahydroazepino[4,5-b]indole- literature compound (patent FR1524495) | 71.7% | −0.8% |
| 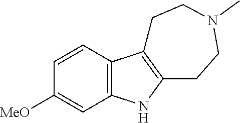 [SMILES: COc1ccc2c3c([nH]c2c1)CCN(C)CC3] 8-methoxy-3-methyl- 1,2,3,4,5,6- hexahydroazepino[4,5-b]indole (TBG)-literature compound (Nature 2021, 589, 474-479) | 18.2% | −2.2% |
| 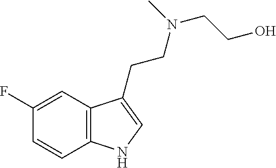 [SMILES: CN(CCO)CCc1c[nH]c2ccc(F)cc12] 2-[2-(5-Fluoro-1H-indol-3- yl)ethyl-methyl-amino]ethanol | 47.1% | 1.2% |
| 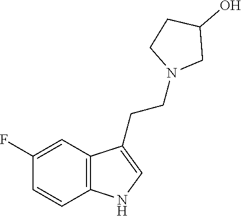 [SMILES: OC1CCN(CCc2c[nH]c3ccc(F)cc23)C1] 1-[2-(5-fluoro-1H-indol-3- yl)ethyl]pyrrolidin-3- olethanamine | 46.0% | −0.7% |
| 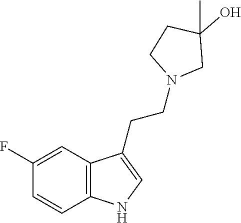 [SMILES: CC1(O)CCN(CCc2c[nH]c3ccc(F)cc23)C1] 1-[2-(5-fluoro-1H-indol-3- yl)ethyl]-3-methyl-pyrrolidin-3- ol | 7.5% | −1.5% |
| 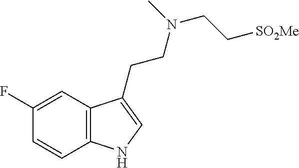 [SMILES: CN(CCc1c[nH]c2ccc(F)cc12)CCS(C)(=O)=O] 2-(5-fluoro-1H-indol-3-yl)-N- methyl-N-(2- methylsulfonylethyl)ethanamine | 27.3% | −0.5% |
| 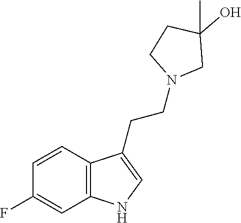 [SMILES: CC1(O)CCN(CCc2c[nH]c3cc(F)ccc23)C1] 1-[2-(6-fluoro-1H-indol-3- yl)ethyl]-3-methyl-pyrrolidin-3- ol | −0.3% | −1.1% |
| 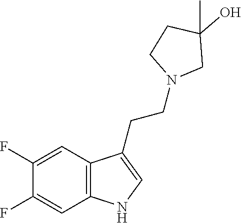 [SMILES: CC1(O)CCN(CCc2c[nH]c3cc(F)c(F)cc23)C1] 1-[2-(5,6-difluoro-1H-indol-3- yl)ethyl]-3-methyl-pyrrolidin-3- ol | 1.9% | −1.0% |
| 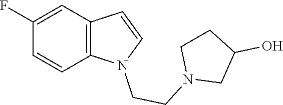 [SMILES: OC1CCN(CCn2ccc3cc(F)ccc32)C1] 1-[2-(5-Fluoroindol-1- yl)ethyl]pyrrolidin-3-ol | −0.2% | −1.2% |
| 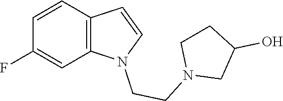 [SMILES: OC1CCN(CCn2ccc3ccc(F)cc32)C1] 1-[2-(6-Fluoroindol-1- yl)ethyl]pyrrolidin-3-ol | 2.5% | −2.8% |
| 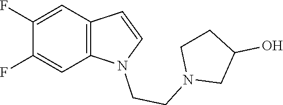 [SMILES: OC1CCN(CCn2ccc3cc(F)c(F)cc32)C1] 1-[2-(5,6-Difluoroindol-1- yl)ethyl]pyrrolidin-3-ol | 2.0% | −0.7% |
| 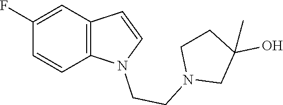 [SMILES: CC1(O)CCN(CCn2ccc3cc(F)ccc32)C1] 1-[2-(5-Fluoroindol-1-yl)ethyl]- 3-methyl-pyrrolidin-3-ol | −0.3% | −1.1% |
| 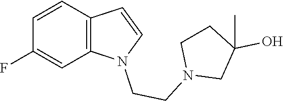 [SMILES: CC1(O)CCN(CCn2ccc3ccc(F)cc32)C1] 1-[2-(6-Fluoroindol-1-yl)ethyl]- 3-methyl-pyrrolidin-3-ol | 0% | −1.3% |
| 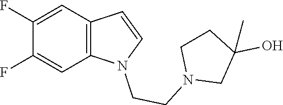 [SMILES: CC1(O)CCN(CCn2ccc3cc(F)c(F)cc32)C1] 1-[2-(5,6-Difluoroindol-1- yl)ethyl]-3-methyl-pyrrolidin-3- ol | −0.1% | −2.5% |
| 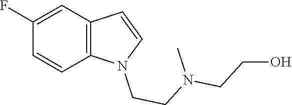 [SMILES: CN(CCO)CCn1ccc2cc(F)ccc21] 2-[2-(5-Fluoroindol-1-yl)ethyl- methyl-amino]ethanol | −0.2% | −1.4% |
| 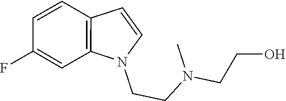 [SMILES: CN(CCO)CCn1ccc2ccc(F)cc21] 2-[2-(6-Fluoroindol-1-yl)ethyl- methyl-amino]ethanol | 8.3% | 0.2% |
| 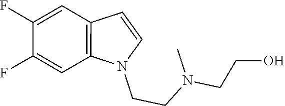 [SMILES: CN(CCO)CCn1ccc2cc(F)c(F)cc21] 2-[2-(5,6-Difluoroindol-1- yl)ethyl-methyl-amino]ethanol | 6.2% | 0.1% |
| 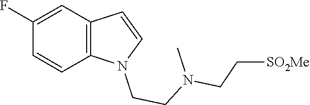 [SMILES: CN(CCn1ccc2cc(F)ccc21)CCS(C)(=O)=O] 2-(5-Fluoroindol-1-yl)-N- methyl-N-(2- methylsulfonylethyl)ethanamine | −0.8% | −2.7% |
| 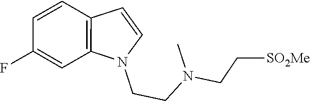 [SMILES: CN(CCn1ccc2ccc(F)cc21)CCS(C)(=O)=O] 2-(6-Fluoroindol-1-yl)-N- methyl-N-(2- methylsulfonylethyl)ethanamine | −1.2% | −1.3% |
| 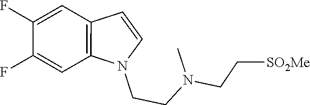 [SMILES: CN(CCn1ccc2cc(F)c(F)cc21)CCS(C)(=O)=O] 2-(5,6-Difluoroindol-1-yl)-N- methyl-N-(2- methylsulfonylethyl)ethanamine | −0.5% | −1.3% |

A compound was deemed to have an advantageous property if it was found to be an agonist, or partial agonist, or antagonist, of the 5-HT_{2A} receptor, when screened at a concentration of 1 μM (one micromolar), or when screened at a higher concentration, (e.g. 10 μM; ten micromolar) or lower concentration (e.g. 100 nM; one hundred nanomolar), whilst also not serving as an agonist of the 5-HT2B receptor (defined as <20% relative efficacy in relation to 5-HT) at a screening concentration of 1 μM (one micromolar). Agonism, or partial agonism, of the 5-HT_{2A} receptor (also known as positive allosteric modulation, or 5-HT_{2A} modulation) is useful for the treatment of neurological and psychiatric disorders. For example, 5-HT_{2A} agonism has been correlated with the promotion of neural plasticity (Ly et al., 2018). Antagonism of the 5HT_{2A} receptor is also useful for the treatment of neurological and psychiatric disorders (Mestre et al., Expert Opinion Investigational Drugs 2013, 22, 411-421). Agonism of the 5-HT2B receptor has been associated with unwanted cardiac valvulopathy side-effects, a form of cardio-toxicity (Rothman et al., Circulation. 2000, 102, 2836-2841; Fitzgerald et al., Molecular Pharmacology 2000, 57, 75-81).

Compounds disclosed herein with desirable properties may serve as agonists, partial agonists, or antagonists of the 5-HT_{2A} receptor, and do not serve as agonists of the 5-HT2B receptor. For research comparative purposes, two literature compounds—8-methoxy-1,2,3,4,5,6-hexahydroazepino[4,5-b]indole—literature compound (patent FRI524495) and 8-methoxy-3-methyl-1,2,3,4,5,6-hexahydroazepino[4,5-b]indole (TBG)—literature compound (Nature 2021, 589, 474-479)—were included in the screening assays. Additionally, 5-hydroxytryptamine/serotonin (5-HT) was included in the screening assay as a positive control agonist. Preferred compounds that were shown to advantageous properties at the 5-HT_{2A} or 5-HT2B receptor as discussed herein include, 2-[2-(5-Fluoro-1H-indol-3-yl)ethyl-methyl-amino]ethanol, 1-[2-(5-fluoro-1H-indol-3-yl)ethyl]pyrrolidin-3-olethanamine, 1-[2-(5-fluoro-1H-indol-3-yl)ethyl]-3-methyl-pyrrolidin-3-ol, 2-(5-fluoro-1H-indol-3-yl)-N-methyl-N-(2-methylsulfonylethyl)ethanamine, 1-[2-(6-fluoro-1H-indol-3-yl)ethyl]-3-methyl-pyrrolidin-3-ol, 1-[2-(5,6-difluoro-1H-indol-3-yl)ethyl]-3-methyl-pyrrolidin-3-ol, 1-[2-(5-Fluoroindol-1-yl)ethyl]pyrrolidin-3-ol, 1-[2-(6-Fluoroindol-1-yl)ethyl]pyrrolidin-3-ol, 1-[2-(5,6-Difluoroindol-1-yl)ethyl]pyrrolidin-3-ol, 1-[2-(5-Fluoroindol-1-yl)ethyl]-3-methyl-pyrrolidin-3-ol, 1-[2-(6-Fluoroindol-1-yl)ethyl]-3-methyl-pyrrolidin-3-ol, 1-[2-(5,6-Difluoroindol-1-yl)ethyl]-3-methyl-pyrrolidin-3-ol, 2-[2-(5-Fluoroindol-1-yl)ethyl-methyl-amino]ethanol, 2-[2-(6-Fluoroindol-1-yl)ethyl-methyl-amino]ethanol, 2-[2-(5,6-Difluoroindol-1-yl)ethyl-methyl-amino]ethanol, 2-(5-Fluoroindol-1-yl)-N-methyl-N-(2-methylsulfonylethyl)ethanamine, 2-(6-Fluoroindol-1-yl)-N-methyl-N-(2-methylsulfonylethyl)ethanamine, and 2-(5,6-Difluoroindol-1-yl)-N-methyl-N-(2-methylsulfonylethyl)ethanamine.

### XI. Evaluation of Metabolic Stability in Human Liver Microsomes

Microsomal Assay: Human liver microsomes (20 mg/mL) are obtained. β-nicotinamide adenine dinucleotide phosphate, reduced form (NADPH), magnesium chloride (MgCl₂), and dimethyl sulfoxide (DMSO) are purchased.

Determination of Metabolic Stability: 7.5 mM stock solutions of test compounds of the above structural formula (e.g., of an embodiment or aspect of embodiment thereof described herein), or pharmaceutically acceptable salt thereof, are prepared in DMSO. The 7.5 mM stock solutions are diluted to 12.5-50 μM in acetonitrile (ACN). The 20 mg/mL human liver microsomes are diluted to 0.625 mg/mL in 0.1 M potassium phosphate buffer, pH 7.4, containing 3 mM MgCl₂. The diluted microsomes are added to wells of a 96-well deep-well polypropylene plate in triplicate. A 10 pL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 4.0 mg/mL human liver microsomes, 0.25 μM test compound, and 2 mM NADPH in 0.1 M potassium phosphate buffer, pH 7.4, and 3 mM MgCl₂. The reaction mixtures are incubated at 37° C., and 50 μL aliquots are removed at 0, 5, 10, 20, and 30 minutes and added to shallow-well 96-well plates which contain 50 μL of ice-cold ACN (acetonitrile) with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-enriched counterpart of the compound and the positive control, 7-ethoxycoumarin (1 μM). Testing is done in triplicate.

Data analysis: The in vitro T_{1/2}s for test compounds are calculated from the slopes of the linear regression of % parent remaining (In) vs incubation time relationship.

### In Vitro T_{1/2}=0.693/k

k=−[slope of linear regression of % parent remaining (In) vs incubation time]

The apparent intrinsic clearance is calculated using the following equation:

CLᵢₙₜ(mL/min/kg)=(0.693/in vitro T)(Incubation Volume/mg of microsomes)(45 mg microsomes/gram of liver)(20 gm of liver/kg b.w.)

Data analysis is performed using Microsoft Excel Software.

In these experiments, values equal to or more than a 15% increase in half-life are considered to be a significant difference.

### XI. Oral Bioavailability in Rats

Pharmacokinetics of test articles following a single intravenous or oral administration in rats: A pharmacokinetic (PK) study is performed in three male Sprague-Dawley (SD) rats following intravenous (IV) and oral (PO) administration of test compounds at 1 mg/kg (IV) and 10 (PO) mg/kg. Test compounds are measured in plasma.

A detailed description of the in vivo methods:

### Rat Strain

Rats used in these studies are supplied by Charles River (Margate UK) and are specific pathogen free. The strain of rats is Sprague Dawley. Male rats are 175-225 g on receipt and are allowed to acclimatise for 5-7 days.

### Animal Housing

Rats are group housed in sterilised individual ventilated cages that expose the animals at all times to HEPA filtered sterile air. Animals will have free access to food and water (sterile) and will have sterile aspen chip bedding (at least once weekly). The room temperature is 22° C.+/−1° C., with a relative humidity of 60% and maximum background noise of 56 dB. Rats are exposed to 12-hour light/dark cycles.

### Treatment

### Treatment

Test article is diluted 10% v/v DMSO, 40% v/v PEG-400, 50% v/v Water. The test articles are administered in a dose volume of 2 mL/kg for intravenous (IV) and 5 mL/kg (PO) for oral routes of administration.

### Single IV/PO Dose Pharmacokinetics Study in Rats

Each test article is administered as a single IV bolus (via a lateral tail-vein) or a single oral gavage in cohorts of 3 rats per route. Following dose administrations, a 100 μL whole blood sample (EDTA) is collected via the tail-vein at time-points described in Table 1. The blood will be centrifuged to separate plasma. Approximately 40 μL of plasma is dispensed per time-point, per rat, in a 96 well plate and frozen until analysis.

### Dose Formulation Samples

Dose formulation samples are diluted in two steps with 50:50 (v/v) methanol/water to an appropriate concentration, then diluted 10:90 (v/v) with control matrix to match to the calibration standard in plasma.

### Sample Extraction Procedure

Calibration and QC standards, incurred samples, blank matrix and dose formulation samples are extracted by protein precipitation, via the addition of a bespoke acetonitrile (ACN)-based Internal Standard (IS) solution, containing several compounds and including Metoprolol and Rosuvastatin, both of which are monitored for during analysis. Following centrifugation, a 40 μL aliquot of supernatant is diluted by the addition of 80 μL water. The prepared sample extracts are analysed by LC-MS/MS.

### Example of Bioanalytical Method and Assay:

| | |
|---|---|
| 1 | According to the plate layout, aliquot to wells in 0.8 mL 96-well plate (Abgene). 30 μL |
| | for Calibration, QC standards, blanks and dose formulation check. |
| 2 | Prepare Calibration and QC standards according to the assay information. Dilute dose |
| | formulation according to the assay information. Aliquot incurred samples according to |
| | the plate layout & assay information. |
| 3 | Add 90 μL of ACN internal standard and vortex mix for 5 minutes at 850 rpm |
| 4 | Centrifuge at nominally 4000 rpm for 10 minutes |
| 6 | Transfer 40 μL of supernatant into a new 0.8 mL Abgene plate. |
| 6 | Add 80 μL of water to all transferred supernatant. |
| 7 | Vortex mix for 30 seconds at 1400 rpm |
| 8 | Analyse immediately by LC-MS/MS or store at +4° C. until analysis. |

### XIII. Biological Assays and Methods

Head-Twitch Response (HTR). The head-twitch response assay is performed as is known to those of skill in the art using both male and female C57BL/6J mice (2 per treatment). The mice are obtained and are approximately 8 weeks old at the time of the experiments. Compounds are administered via intraperitoneal injection (5 mL/kg) using 0.9% saline as the vehicle. As a positive control, 5-MeO-DMT fumarate (2:1 amine/acid) is utilized. Behavior is videotaped, later scored by two blinded observers, and the results are averaged (Pearson correlation coefficient=0.93).

Serotonin and Opioid Receptor Functional Assays. Functional assay screens at 5-HT and opioid receptors are performed in parallel using the same compound dilutions and 384-well format high-throughput assay platforms. Assays assess activity at all human isoforms of the receptors, except where noted for the mouse 5-HT_{2A} receptor. Receptor constructs in pcDNA vectors are generated from the Presto-Tango GPCR library with minor modifications. All compounds are serially diluted in drug buffer (HBSS, 20 mM HEPES, pH 7.4 supplemented with 0.1% bovine serum albumin and 0.01% ascorbic acid) and dispensed into 384-well assay plates using a FLIPR^{TETRA} (Molecular Devices). Every plate included a positive control such as 5-HT (for all 5-HT receptors), DADLE (DOR), salvinorin A (KOR), and DAMGO (MOR). For measurements of 5-HT2A, 5-HT2B, and 5-HT2C Gq-mediated calcium flux function, HEK Flp-In 293 T-Rex stable cell lines (Invitrogen) are loaded with Fluo₄ dye for one hour, stimulated with compounds and read for baseline (0-10 seconds) and peak fold-over-basal fluorescence (5 minutes) at 25° C. on the FLIPR^{TETRA}. For measurement of 5-HT6 and 5-HT7a functional assays, Gs-mediated cAMP accumulation is detected using the split-luciferase GloSensor assay in HEKT cells measuring luminescence on a Microbeta Trilux (Perkin Elmer) with a 15 min drug incubation at 25° C. For 5-HT1A, 5-HT1B, 5-HT1F, MOR, KOR, and DOR functional assays, Gi/o-mediated cAMP inhibition is measured using the split-luciferase GloSensor assay in HEKT cells, conducted similarly as above, but in combination with either 0.3 μM isoproterenol (5-HT1A, 5-HT1B, 5-HT1F) or 1 μM forskolin (MOR, KOR, and DOR) to stimulate endogenous cAMP accumulation. For measurement of 5-HT1D, 5-HT1E, 5-HT4, and 5-HT5A functional assays, P-arrestin2 recruitment is measured by the Tango assay utilizing HTLA cells expressing TEV fused-P-arrestin2, as described previously with minor modifications. Data for all assays are plotted and non-linear regression is performed using “log(agonist) vs. response” in Graphpad Prism to yield Emax and EC₅₀ parameter estimates.

5HT_{2A} Sensor Assays. HEK293T (ATCC) 5HT_{2A} sensor stable line (sLightl.3s) is generated via lentiviral transduction of HIV-EF1α-sLightl.3 and propagated from a single colony. Lentivirus is produced using 2^{nd} generation lentiviral plasmids pHIV-EF1α-sLightl.3, pHCMV-G, and pCMV-deltaR8.2.

For the screening of the compounds, sLightl.3s cells are plated in 96-well plates at a density of 40000 24-hours prior to imaging. On the day of imaging, compounds solubilized in DMSO are diluted from the 100 mM stock solution to working concentrations of 1 mM, 100 mM and 1 μM with a DMSO concentration of 1%. Immediately prior to imaging, cells growing in DMEM (Gibco) are washed 2× with HBSS (Gibco) and in agonist mode 180 μL of HBSS or in antagonist mode 160 μL of HBSS is added to each well after the final wash. For agonist mode, images are taken before and after the addition of the 20 μL compound working solution into the wells containing 180 μL HBSS. This produces final compound concentrations of 100 mM, 10 mM and 100 nM with a DMSO concentration of 0.1%. For antagonist mode, images are taken before and after addition of 20 μL of 900 nM 5-HT and again after 20 μL of the compound working solutions to produce final concentrations of 100 nM for 5HT and 100 mM, 10 mM and 100 nM for the compounds with a DMSO concentration of 0.1%. Each compound is tested in triplicate (3 wells) for each concentration (100 mM, 10 mM and 10 nM). Additionally, within each plate, 100 nM 5HT and 0.1% DMSO controls are also imaged.

Imaging is performed using the Leica DMi8 inverted microscope with a 40× objective using the FITC preset with an excitation of 460 nm and emission of 512-542 nm. For each well, the cellular membrane where the 5HT_{2A} sensor is targeted is autofocused using the adaptive focus controls and 5 images from different regions within the well are taken with each image processed from a 2×2 binning.

For data processing, the membranes from each image are segmented and analyzed using a custom algorithm written in MATFAB producing a single raw fluorescence intensity value. For each well the 5 raw fluorescence intensity values generated from the 5 images are averaged and the change in fluorescence intensity (dFF) is calculated as:

$$dFF = ( {F}_{sat} - {F}_{apo} ) / {F}_{apo}$$

For both agonist and antagonist modes, the fluorescence intensity values before compound addition in FIBSS only are used as the Fₐₚₒ values while the fluorescence intensity values after compound addition are used as the Fₛₐₜ values.

For agonist mode, data are as percent activation relative to 5HT, where 0 is the average of the DMSO wells and 100 is the average of the 100 μM 5HT wells. For antagonist mode, the inactivation score is calculated as:

$$Inactivation score = ( dFFF ( Compund + 5 HT ) - dFF ( 5 HT ) ) / dFF ( 5 HT )$$

Plasticity Effects: Treatment of rat embryonic cortical neurons with compounds of Table 1 or Formulas (I, II, III, IV, V, I.a, V.a) is evaluated for increased dendritic arbor complexity at 6 days in vitro (DIV6) as measured by Sholl analysis. The effect of the present compounds on dendritic growth can be determined to be 5-HT2A-dependent, if pretreatment with ketanserin—a 5-HT2A antagonist—inhibits their effects.

In addition to promoting dendritic growth, the present compounds also are evaluated for increased dendritic spine density to a comparable extent as ibogaine in mature cortical cultures (DIV20). The effects of the compounds on cortical dendritic spine dynamics in vivo using transcranial 2-photon imaging are assessed. First, spines are imaged on specific dendritic loci defined by their relation to blood vessel and dendritic architectures. Next, the animals are systemically administered vehicle, a compound of the present invention, or the hallucinogenic 5-HT2A agonist 2,5-dimethoxy-4-iodoamphetamine (DOI). After 24 hours, the same dendritic segments are re-imaged, and the number of spines gained or lost is quantified. Examples of the presently disclosed compounds increase spine formation in mouse primary sensory cortex, suggesting that the present compounds support neuronal plasticity.

As increased cortical structural plasticity in the anterior parts of the brain mediates the sustained (>24 h) antidepressant-like effects of ketamine and play a role in the therapeutic effects of 5-HT2A agonists, the impact of the present compounds on forced swim test (FST) behavior is evaluated. First, a pretest is used to induce a depressive phenotype. Compounds are administered 24 hours after the pre-test, and the FST is performed 24 h and 7 d post compound administration. Effective compounds of the invention, like ketamine, significantly reduce immobility 24 hours after administration.

Dendritogenesis Assays. Compounds disclosed herein are evaluated for their ability to increase dendritic arbor complexity in cultures of cortical neurons using a phenotypic assay. Following treatment, neurons are fixed and visualized using an antibody against MAP2—a cytoskeletal protein localized to the somatodendritic compartment of neurons. Sholl analysis is then performed, and the maximum number of crossings (Nₘₐₓ) is used as a quantitative metric of dendritic arbor complexity. For statistical comparisons between specific compounds, the raw Nₘₐₓ values are compared. Percent efficacies are determined by setting the Nₘₐₓ values for the vehicle (DMSO) and positive (ketamine) controls equal to 0% and 100%, respectively.

Animals. For the dendritogenesis experiments, timed pregnant Sprague Dawley rats are obtained. For the head-twitch response assay, male and female C57BL/6J mice are obtained.

Dendritogenesis—Sholl Analysis. Dendritogenesis experiments are performed following previously published methods with slight modifications. Neurons are plated in 96-well format (200 μL of media per well) at a density of approximately 15,000 cells/well in Neurobasal (Life Technologies) containing 1% penicillin-streptomycin, 10% heat-inactivated fetal bovine serum, and 0.5 mM glutamine. After 24 hours, the medium is replaced with Neurobasal containing 1× B27 supplement (Life Technologies), 1% penicillin-streptomycin, 0.5 mM glutamine, and 12.5 μM glutamate. After 3 days in vitro (DIV3), the cells are treated with compounds. All compounds tested in the dendritogenesis assays are treated at 10 μM. Stock solutions of the compounds in DMSO are first diluted 100-fold in Neurobasal before an additional 10-fold dilution into each well (total dilution=1:1000; 0.1% DMSO concentration). Treatments are randomized. After 1 hour, the media is removed and replaced with new Neurobasal media containing 1× B27 supplement, 1% penicillin-streptomycin, 0.5 mM glutamine, and 12.5 mM glutamate. The cells are allowed to grow for an additional 71 hours. At that time, neurons are fixed by removing 80% of the media and replacing it with a volume of 4% aqueous paraformaldehyde (Alfa Aesar) equal to 50% of the working volume of the well. Then, the cells are incubated at room temperature for 20 minutes before the fixative is aspirated and each well washed twice with DPBS. Cells are permeabilized using 0.2% Triton X-100 (ThermoFisher) in DPBS for 20 minutes at room temperature without shaking. Plates are blocked with antibody diluting buffer (ADB) containing 2% bovine serum albumin (BSA) in DPBS for 1 hour at room temperature. Then, plates are incubated overnight at 4° C. with gentle shaking in ADB containing a chicken anti-MAP2 antibody (1:10,000; EnCor, CPCA-MAP2). The next day, plates are washed three times with DPBS and once with 2% ADB in DPBS. Plates are incubated for 1 hour at room temperature in ADB containing an anti-chicken IgG secondary antibody conjugated to Alexa Fluor 488 (Life Technologies, 1:500) and washed five times with DPBS. After the final wash, 100 μL of DPBS is added per well and imaged on an ImageXpress Micro XL High-Content Screening System (Molecular Devices, Sunnyvale, CA) with a 20× objective. Images are analyzed using ImageJ Fiji (version 1.51 W). First, images corresponding to each treatment are sorted into individual folders that are then blinded for data analysis. Plate controls (both positive and negative) are used to ensure that the assay is working properly as well as to visually determine appropriate numerical values for brightness/contrast and thresholding to be applied universally to the remainder of the randomized images. Next, the brightness/contrast settings are applied, and approximately 1-2 individual pyramidal-like neurons per image (i.e., no bipolar neurons) are selected using the rectangular selection tool and saved as separate files. Neurons are selected that do not overlap extensively with other cells or extend far beyond the field of view.

In Vivo Spine Dynamics. Male and female Thy1-GFP-M line mice (n=5 per condition) are maintained. In vivo transcranial two-photon imaging and data analysis are performed as previously described. Briefly, mice are anesthetized with an intraperitoneal (i.p.) injection of a mixture of ketamine (87 mg/kg) and xylazine (8.7 mg/kg). A small region of the exposed skull is manually thinned down to 20-30 pm for optical access. Spines on apical dendrites in mouse primary sensory cortices are imaged using a Bruker Ultima IV two-photon microscope equipped with an Olympus water-immersion objective (40×, NA=0.8) and a Ti:Sapphire laser (Spectra-Physics Mai-Tai, excitation wavelength 920 nm). Images are taken at a zoom of 4.0 (pixel size 0.143×0.143 pm) and Z-step size of 0.7 pm. The mice receive an i.p. injection (injection volume=5 mL/kg) of DOI (10 mg/kg) or test compound (50 mg/kg) immediately after they recover from anesthesia given prior to the first imaging session. The animals are re-imaged 24 hours after drug administration. Dendritic spine dynamics are analyzed using ImageJ. Spine formation and elimination are quantified as percentages of spine number on day 0.

Forced Swim Test (FST). Male C57/BL6J mice (9-10 weeks old at time of experiment) are obtained. After 1 week in the vivarium each mouse is handled for approximately 1 minute by the experimenter for 3 consecutive days leading up to the first FST. All experiments are carried out by the same experimenter who performs handling. During the FST, mice undergo a 6 minute swim session in a clear Plexiglas cylinder 40 cm tall, 20 cm in diameter, and filled with 30 cm of 24±1° C. water. Fresh water is used for every mouse. After handling and habituation to the experimenter, drug-naive mice first undergo a pretest swim to more reliably induce a depressive phenotype in the subsequent FST sessions. Immobility scores for all mice are determined after the pre-test and mice are randomly assigned to treatment groups to generate groups with similar average immobility scores to be used for the following two FST sessions. The next day, the animals receive intraperitoneal injections of experimental compounds (20 mg/kg), a positive control (ketamine, 3 mg/kg), or vehicle (saline). The animals are subjected to the FST 30 mins after injection and then returned to their home cages. All FSTs are performed between the hours of 8 am and 1 pm. Experiments are video-recorded and manually scored offline. Immobility time—defined as passive floating or remaining motionless with no activity other than that needed to keep the mouse's head above water—is scored for the last 4 minutes of the 6 minute trial.

Statistical analysis. Treatments are randomized, and data are analyzed by experimenters blinded to treatment conditions. Statistical analyses are performed using GraphPad Prism (version 8.1.2). The specific tests are, F-statistics and degrees of freedom. All comparisons are planned prior to performing each experiment. For dendritogenesis experiments a one way ANOVA with Dunnett's post hoc test is deemed most appropriate. Ketamine is included as a positive control to ensure that the assay is working properly.

Alcohol Use Disorder Model: To assess the anti-addictive potential of the present compounds, an alcohol drinking paradigm that models heavy alcohol use and binge drinking behavior in humans is employed. Using a 2-bottle choice setup (20% ethanol (v/v), EtOH vs. water, H₂O), mice are subjected to repeated cycles of binge drinking and withdrawal over the course of 7 weeks.

This schedule results in heavy EtOH consumption, binge drinking-like behavior, and generates blood alcohol content equivalent to that of human subjects suffering from alcohol use disorder (AUD). Next, compounds of the invention are administered via intraperitoneal injection 3 hours prior to a drinking session, and EtOH and H₂O consumption is monitored. Effective compounds of the invention robustly reduce binge drinking during the first 4 hours, decreasing EtOH consumption. With exemplary compounds, consumption of ethanol is lower for at least two days following administration with no effect on water intake. Efficacy in this assay suggests the present compounds are useful for treating AUD.

### XIV. Making Disclosed Compounds

Compounds according to Table 1 or Formulas I-IV may be prepared using known chemical reactions and procedures, some from starting materials which are commercially available. Nevertheless, the following general preparative methods are presented to further aid one of ordinary skill in the art to synthesize such compounds.

Scheme I provides an exemplary synthesis protocol particularly suitable for making compounds according to Formula (II) where Z is —OH. Such compounds can be prepared by acylation of a suitably substituted indole 10 with an appropriate acylating agent, such as oxalyl chloride 12, followed by treatment with phthalimide 14 and then an appropriately substituted amino alcohol 16 to produce glyoxamide 18. Glyoxamide 18 can then be reduced with an appropriate reducing agent, such as a hydride reducing agent, including lithium aluminum hydride or borane, to produce compound 20.

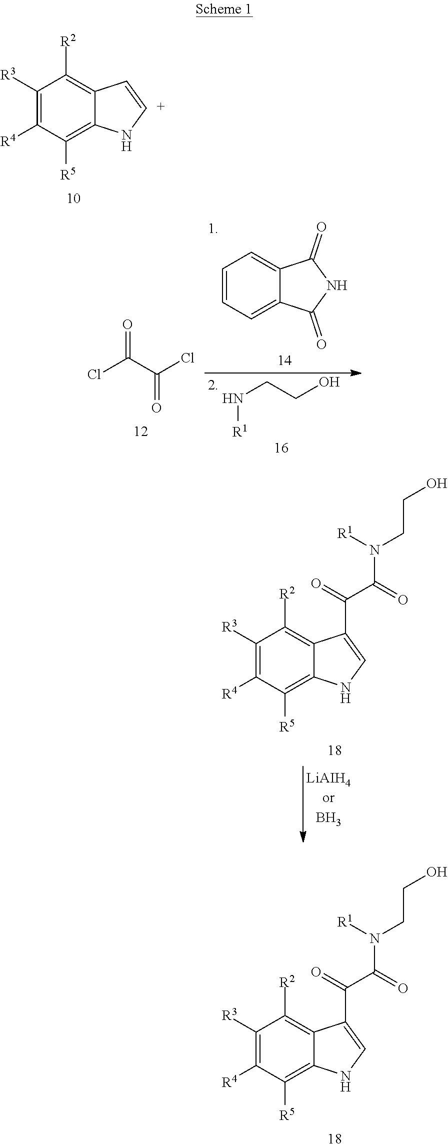

A similar procedure for preparing sulfides (Z=—SR^{7a}) and sulfoxides (Z=—S(O)R^{7a}) according to Formula (II) is illustrated by Scheme 2. Acylation of a suitably substituted indole 10 with an appropriate acylating agent, such as oxalyl chloride 12, followed by treatment with phthalimide 14 and an appropriately substituted amino sulfide 22, produces glyoxamide 24. Glyoxamide 24 can then be reduced with an appropriate reducing agent, such as a hydride reducing agent, including lithium aluminum hydride or borane, to produce sulfide 26. Alternatively, intermediate glyoxamide 24 can be oxidized using a suitable oxidizing reagent, such as sodium periodate or iodosobenzene, followed by reduction to provide sulfoxide 28 (Z=—S(O)R^{7a}).

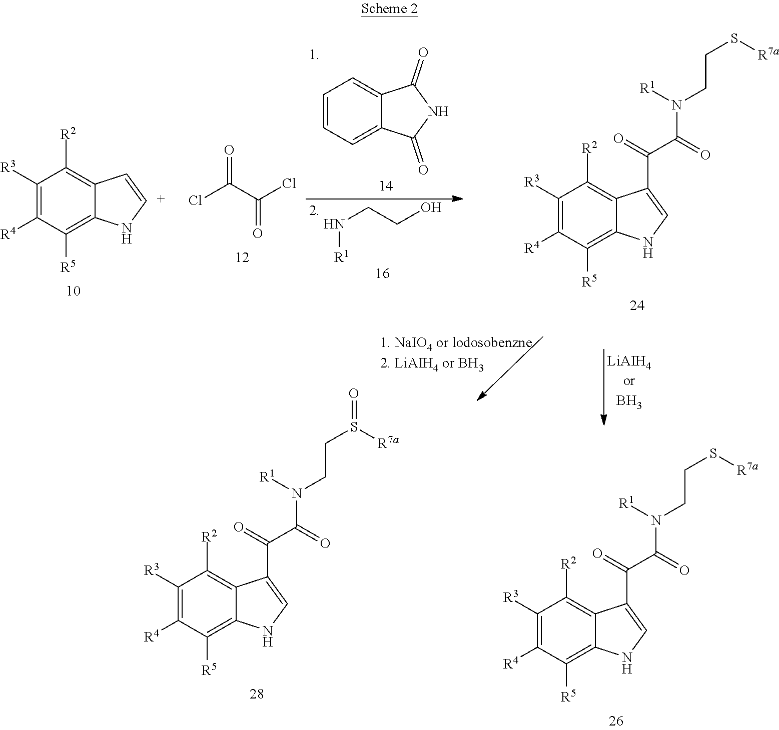

As yet another alternative, intermediate glyoxamide 24 can be used to produce sulfones (Z=—S(O)₂R^{7a}) as illustrated by Scheme 3. That is, intermediate glyoxamide 24 can be oxidized with a suitable oxidizing agent, such as a peracid, with one example being 3-chloroperbenzoic acid. The oxidation reaction is then followed by reduction to provide sulfone 30 (Z=—S(O)₂R^{7a}) as illustrated by Scheme 3.

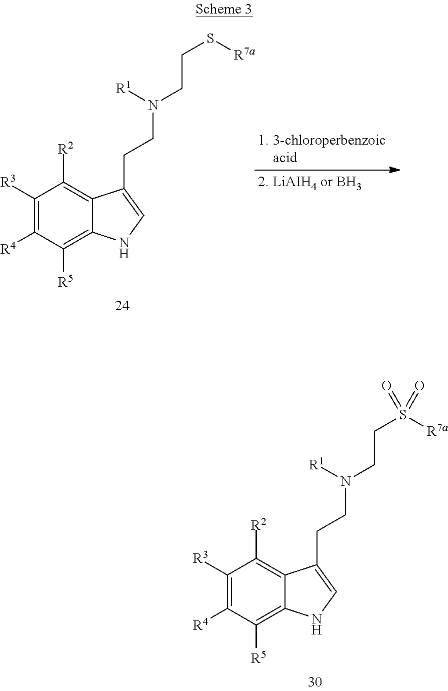

The amines (Z=—N(R^{7b}R^{7c}) of Formula (II) in which R^{7b} and R^{7c} are both C₁₋₆ alkyl can be prepared similarly as shown by Scheme 4. With reference to Scheme 4, acylation of a suitably substituted indole 10 with an appropriate acylating agent, such as oxalyl chloride 12, followed by treatment with phthalimide 14 and an appropriately substituted diamine 32, produces glyoxamide 34. Glyoxamide 34 can then be reduced with an appropriate reducing agent, such as a hydride reducing agent, including lithium aluminum hydride or borane, to produce diamine 36.

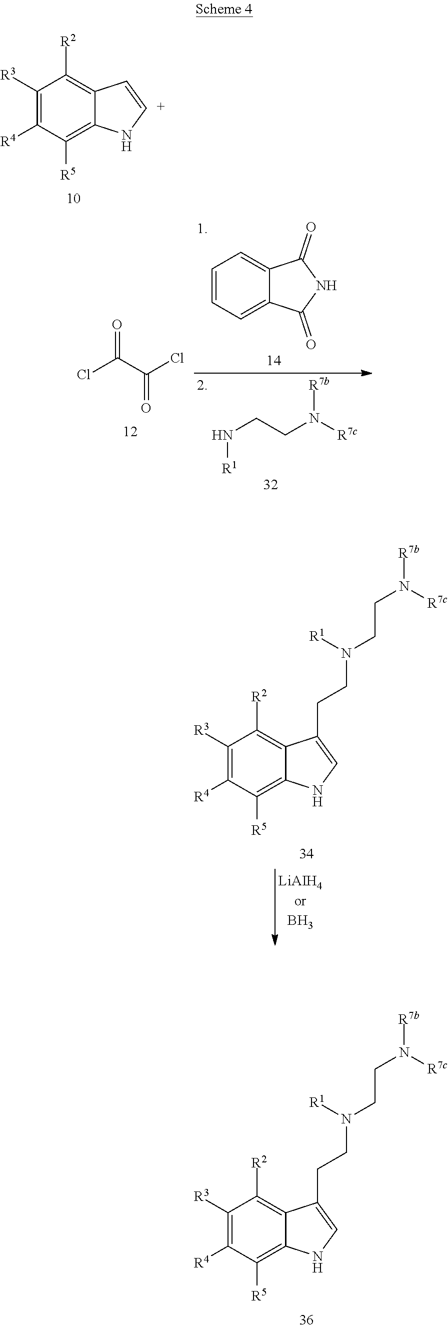

Amines (Z=—N(R^{7b}R^{7c}) in which both R^{7b} and R^{7c} are H, or if one is H and the other is C₁₋₆ alkyl, can be prepared according to Scheme 5. With reference to Scheme 5, acylation of a suitably substituted indole 10 with an appropriate acylating agent, such as oxalyl chloride 12, followed by treatment with phthalimide 14 and an appropriately substituted compound 40, produces glyoxamide 42. Gyloxamide 42 includes a suitable amine protecting group, such as the illustrated t-butyloxycarbonyl protecting group. The t-butyloxycarbonyl protecting group is removed using strong acid, such as trifluoroacetic acid. Diamine 46 is then produced using a suitable reducing agent, such as a hydride reducing agent, including lithium aluminum hydride or borane.

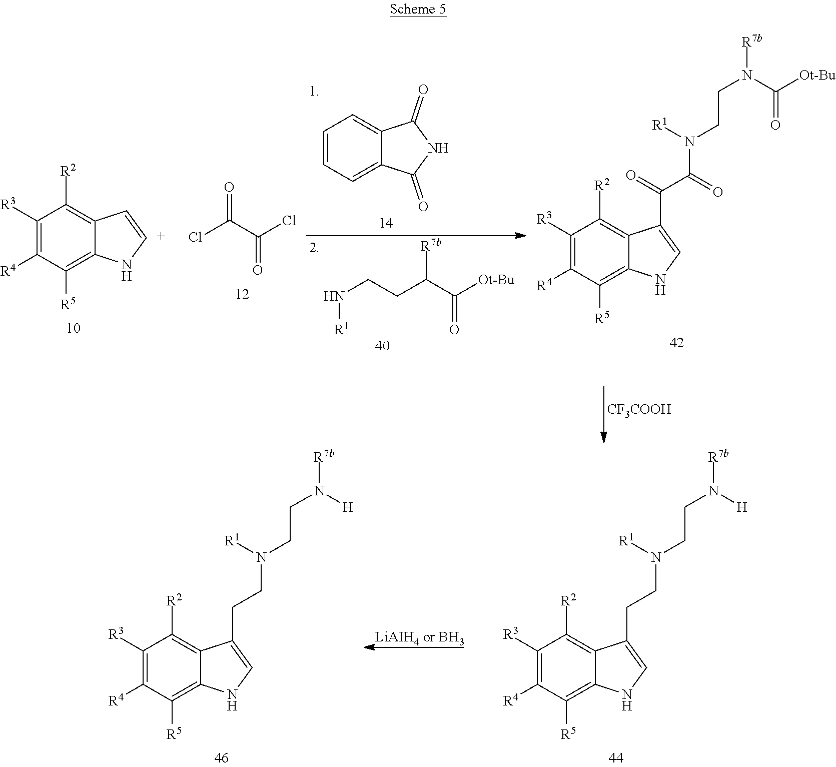

The final product 46 from Scheme 5 can be used to prepare compounds of Formula (II) in which Z is —N(R^{7b})C(O)R^{7c} according to Scheme 6b, such as compound 48. Amine 46 can be acylated using any suitable acylating agent, such as the illustrated acid chloride, or an acid anhydride. Alternatively, amine 46 can be acylated with a carboxylic acid in the presence of a suitable coupling reagent, such as a carbodiimide.

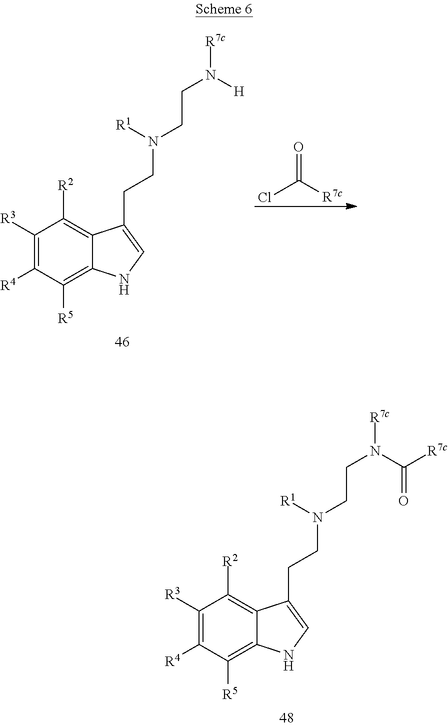

Alternatively, final products can be synthesized as outlined by the Schemes, or processes, illustrated in the Experimental Procedures section.

### Experimental Procedures

General Method 1: N-alkylation of fluoroindoles

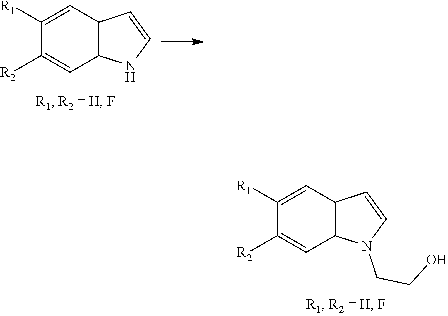

To a stirred solution of the appropriate fluoroindole (15 mmol) in DMF (35 mL) at rt was added K₂CO₃ (22.5 mmol) followed by 1.3-dioxolan-2-one (60 mmol). The mixture was heated to 100° C. (oil bath temperature) and stirred for 24 h. Additional 1,3-dioxolan-2-one (34.5 mmol) was added and heating continued at 100° C. for 23 h. The mixture was cooled to rt, H₂O (250 mL) was added and the mixture was extracted with Et₂O (2×50 mL). The combined organic layers were washed sequentially with H₂O (2×200 mL) and brine (200 mL), dried over MgSO₄, filtered and concentrated in vacuo. The crude product was purified by column chromatography on silica gel (eluent: 0-100% EtOAc in hexane) to give the product.

### 2-(6-fluoroindol-1-yl)ethanol

Prepared using the general method 1. The title compound was isolated as a viscous oil (1.75 g, 66%). Retention time 1.52 min; m/z calculated for [C₁₀H₁₀FNO]⁺ 179.1. Found 180.2 [M+H]⁺; ¹H NMR (400 MHz, CDCl₃) δ 7.52 (dd, J=8.7, 5.4 Hz, 1H), 7.12 (d, J=3.2 Hz, 1H), 7.04 (dd, J=9.9, 2.3 Hz, 1H), 6.88 (ddd, J=9.6, 8.6, 2.3 Hz, 1H), 6.48 (dd, J=3.2, 0.9 Hz, 1H), 4.20 (dd, J=5.7, 4.8 Hz, 2H), 3.93 (td, J=5.8, 4.8 Hz, 2H), 1.57 (t, J=6.0 Hz, 1H).

### 2-(5-fluoroindol-1-yl)ethanol

Prepared using the general method 1. The title compound was isolated as a viscous oil (2.12 g, 79%). Retention time 1.50 min; m/z calculated for [C₁₀H₁₀FNO]⁺ 179.1. Found 180.0 [M+H]⁺; ¹H NMR (400 MHz, CDCl₃) δ 7.30-7.22 (m, 2H), 7.18 (d, J=3.1 Hz, 1H), 6.95 (td, J=9.0, 2.4 Hz, 1H), 6.46 (dd, J=3.1, 0.8 Hz, 1H), 4.24 (dd, J=5.6, 4.9 Hz, 2H), 3.97-3.88 (m, 2H), 1.57 (t, J=6.0 Hz, 1H).

### 2-(5,6-Difluoroindol-1-yl)ethanol

To a stirred solution of 5,6-difluoro-1H-indole (2.0 g, 13.1 mmol) in DMF (40 mL) at rt was added K₂CO₃ (2.71 g, 19.6 mmol) followed by 1,3-dioxolan-2-one (4.60 g, 52.2 mmol). The reaction mixture was heated to 90° C. and stirred for 15 h. The mixture was allowed to cool, poured onto ice-H₂O (500 mL) and extracted with Et₂O (3×75 mL). The combined organic layers were washed with 5% aq LiCl (2×50 mL) and concentrated in vacuo. The crude residue was purified by column chromatography on silica gel (eluent: 10 to 50% EtOAc in hexane) to afford the title compound as a viscous oil (1.51 g, 58%). Retention time 1.53 min; m/z calculated for [C₁₀H₉F₂NO]⁺ 198.0. Found 198.0 [M+H]⁺; ¹H NMR (400 MHz, d₆-DMSO) δ 7.34 (dd, J=10.7, 7.8 Hz, 1H), 7.20-7.10 (m, 2H), 6.46 (dd, J=3.2, 0.9 Hz, 1H), 4.21 (dd, J=5.6, 4.9 Hz, 2H), 3.96 (td, J=5.7, 4.8 Hz, 2H), 1.50 (t, J=5.8 Hz, 1H).

### General Method 2: Mesylation of Alcohol

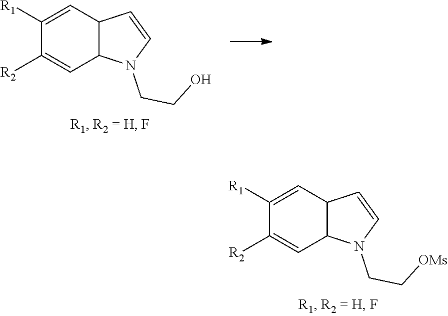

To a stirred mixture of the appropriate indol-1-ylethan-1-ol (10 mmol) and Et₃N (12.5 mmol) in DCM (30 mL) was added dropwise a solution of methanesulfonyl chloride (11 mmol) in DCM (25 mL) over 30 min. The mixture was stirred overnight, then H₂O (30 mL) was added, the mixture separated and extracted with DCM (2×20 mL). The combined organic layers were washed in turn with H₂O (30 mL) and brine (30 mL), dried over MgSO₄, filtered and concentrated in vacuo. The mesylate product was used in the next step without further purification.

### 2-(6-Fluoroindol-1-yl)ethyl methanesulfonate

Prepared using general method 2, using 1.75 g (9.77 mmol) of 2-(6-fluoroindol-1-yl)ethanol reactant. The title compound was isolated as a solid (1.49 g, 59%). Retention time 1.62 min; m/z calculated for [C₁₁H₁₂FNO₃S]⁺ 257.2. Found 258.0 [M+H]⁺; ¹H NMR (400 MHz, CDCl₃) δ 7.53 (dd, J=8.7, 5.4 Hz, 1H), 7.11 (d, J=3.3 Hz, 1H), 7.03 (dd, J=9.7, 2.3 Hz, 1H), 6.90 (ddd, J=9.6, 8.7, 2.3 Hz, 1H), 6.52 (dd, J=3.3, 0.9 Hz, 1H), 4.53-4.45 (m, 2H), 4.45-4.36 (m, 2H), 2.67 (s, 3H).

### 2-(5-Fluoroindol-1-yl)ethyl methanesulfonate

Prepared using general method 2, using 2.12 g (11.8 mmol) of 2-(5-fluoroindol-1-yl)ethanol reactant. The title compound was isolated as a solid (2.53 g, 83%). Retention time 1.61 min; m/z calculated for [C₁₁H₁₂FNO₃S]⁺ 257.2. Found 258.0 [M+H]⁺; ¹H NMR (400 MHz, CDCl₃) δ 7.31-7.19 (m, 2H), 7.16 (s, 1H), 6.99 (tdd, J=9.2, 2.5, 0.5 Hz, 1H), 6.49 (dd, J=3.2, 0.8 Hz, 1H), 4.53-4.39 (m, 4H), 2.63 (s, 3H).

### 2-(5,6-Difluoroindol-1-yl)ethyl methanesulfonate

Prepared using general method 2, using 1.19 g (6.04 mmol) of 2-(5,6-difluoroindol-1-yl)ethanol reactant. The title compound was isolated as a solid (1.53 g, 92%). Retention time 1.64 min; m/z calculated for [C₁₁H₁₁F₂NO₃S]⁺ 275.2. Found 276.0 [M+H]⁺; ¹H NMR (400 MHz, d₆-DMSO) δ 7.66 (ddd, J=11.7, 7.0, 0.9 Hz, 1H), 7.53 (dd, J=11.3, 8.0 Hz, 1H), 7.46 (dd, J=3.2, 0.6 Hz, 1H), 6.48 (dd, J=3.2, 0.8 Hz, 1H), 4.56-4.45 (m, 4H), 2.99 (s, 3H).

### General Method 3: Displacement with pyrrolidine-3-ol

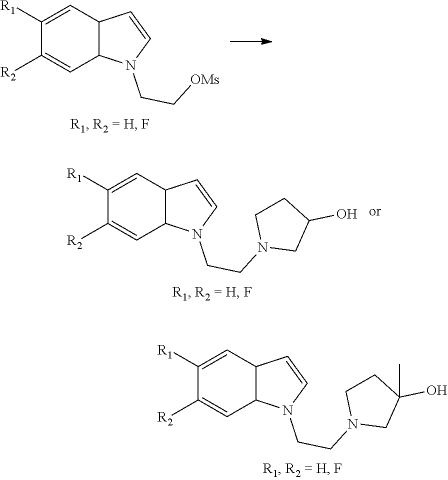

A suspension of the mesylate (2 mmol), K₂CO₃ (2.1 mmol) and racemic pyrrolidine-3-ol (3 mmol) in acetonitrile (6 mL) was heated to 60° C. (oil bath temperature) and stirred for 20 h. The mixture was cooled to rt, H₂O (20 mL) was added and the mixture was extracted with DCM (2×14 mL). The combined organic layers were washed with brine (14 mL), concentrated in vacuo and purified by column chromatography on silica gel (eluent: 10% MeOH in DCM).

### 1-[2-(6-Fluoroindol-1-yl)ethyl]pyrrolidin-3-ol

Prepared using general method 3, using 2-(6-fluoroindol-1-yl)ethyl methanesulfonate (0.40 g, 1.55 mmol). The title compound was isolated as a solid (266 mg, 68%). Retention time 1.57 mins (10 minute LCMS method); m/z calculated for [C₁₄H₁₇FN₂O]⁺ 249.1. Found 249.0 [M+H]⁺; ¹H NMR (400 MHz, d₆-DMSO) δ 7.51 (dd, J=8.6, 5.5 Hz, 1H), 7.37 (d, J=3.2 Hz, 1H), 7.36-7.27 (m, 1H), 6.85 (ddd, J=9.8, 8.6, 2.4 Hz, 1H), 6.42 (dd, J=3.2, 0.9 Hz, 1H), 4.61 (d, J=4.5 Hz, 1H), 4.20 (q, J=7.3 Hz, 2H), 4.17-4.10 (m, 1H), 2.81-2.69 (m, 3H), 2.65-2.55 (m, 1H), 2.51-2.43 (m, 1H), 2.34 (dd, J=9.7, 3.9 Hz, 1H), 1.92 (dtd, J=12.8, 7.7, 6.7 Hz, 1H), 1.52 (dddd, J=13.0, 7.7, 5.4, 3.5 Hz, 1H).

### 1-[2-(5-Fluoroindol-1-yl)ethyl]pyrrolidin-3-ol

Prepared using general method 3, using 2-(5-fluoroindol-1-yl)ethyl methanesulfonate (0.40 g, 1.55 mmol). The title compound was isolated as a viscous oil (213 mg, 55%). Retention time 1.11 min; m/z calculated for [C₁₄H₁₇FN₂O]⁺ 249.1. Found 249.2 [M+H]⁺; ¹H NMR (400 MHz, d₆-DMSO) δ 7.50-7.42 (m, 2H), 7.28 (dd, J=9.9, 2.5 Hz, 1H), 6.95 (td, J=9.2, 2.6 Hz, 1H), 6.40 (dd, J=3.1, 0.8 Hz, 1H), 4.60 (d, J=4.5 Hz, 1H), 4.24 (t, J=6.7 Hz, 2H), 4.15 (ddq, J=7.6, 6.0, 3.8 Hz, 1H), 2.82-2.69 (m, 3H), 2.59 (dt, J=8.8, 7.0 Hz, 1H), 2.49-2.42 (m, 1H), 2.33 (dd, J=9.6, 3.9 Hz, 1H), 1.92 (dtd, J=12.8, 7.7, 6.8 Hz, 1H), 1.51 (dddd, J=13.0, 7.8, 5.5, 3.5 Hz, 1H).

### 1-[2-(5,6-Difluoroindol-1-yl)ethyl]pyrrolidin-3-ol

Prepared using general method 3, using 2-(5,6-difluoroindol-1-yl)ethyl methanesulfonate (0.40 g, 1.45 mmol). The title compound was isolated as a solid (260 mg, 67%). Retention time 1.15 min; m/z calculated for [C₁₄H₁₆F₂N₂O]⁺ 267.1. Found 267.2 [M+H]⁺; ¹H NMR (400 MHz, d₆-DMSO) δ 7.58 (ddd, J=11.7, 7.0, 0.8 Hz, 1H), 7.49 (dd, J=11.3, 8.1 Hz, 1H), 7.45 (d, J=3.1 Hz, 1H), 6.42 (dd, J=3.2, 0.8 Hz, 1H), 4.60 (d, J=4.5 Hz, 1H), 4.25-4.11 (m, 3H), 2.82-2.68 (m, 3H), 2.64-2.54 (m, 1H), 2.50-2.41 (m, 1H), 2.33 (dd, J=9.7, 3.9 Hz, 1H), 1.92 (dtd, J=12.8, 7.7, 6.7 Hz, 1H), 1.51 (dddd, J=13.0, 7.7, 5.4, 3.5 Hz, 1H).

### 1-[2-(6-Fluoroindol-1-yl)ethyl]-3-methyl-pyrrolidin-3-ol

Prepared using general method 3, using 2-(6-fluoroindol-1-yl)ethyl methanesulfonate (0.340 g, 1.32 mmol). The title compound was isolated as a viscous oil (0.103 g, 29%). Retention time 1.13 min; m/z calculated for [C₁₅H₁₉FN₂O]⁺ 262.3. Found 263.2 [M+H]⁺; ¹H NMR (400 MHz, CDCl₃) δ 7.51 (dd, J=8.3, 5.6 Hz, 1H), 7.10 (d, J=3.2 Hz, 1H), 7.01 (ddt, J=9.9, 2.4, 0.7 Hz, 1H), 6.86 (ddd, J=9.6, 8.6, 2.3 Hz, 1H), 6.46 (dd, J=3.2, 0.9 Hz, 1H), 4.18 (t, J=6.9 Hz, 2H), 3.00 (dt, J=9.1, 6.8 Hz, 1H), 2.95-2.80 (m, 2H), 2.72 (dt, J=9.3, 0.8 Hz, 1H), 2.36 (td, J=8.8, 7.2 Hz, 1H), 2.28 (d, J=9.2 Hz, 1H), 1.92-1.81 (m, 2H), 1.34 (s, 3H).

### 1-[2-(5-Fluoroindol-1-yl)ethyl]-3-methyl-pyrrolidin-3-ol

Prepared using general method 3, using 2-(5-fluoroindol-1-yl)ethyl methanesulfonate (0.40 g, 1.55 mmol). The title compound was isolated as a viscous oil (254 mg, 62%). Retention time 1.14 min; m/z calculated for [C₁₅H₁₉FN₂O]⁺ 263.1. Found 263.2 [M+H]⁺; ¹H NMR (400 MHz, d₆-DMSO) δ 7.46 (dd, J=9.0, 4.0 Hz, 2H), 7.28 (dd, J=9.9, 2.5 Hz, 1H), 6.95 (td, J=9.3, 2.6 Hz, 1H), 6.40 (dd, J=3.1, 0.8 Hz, 1H), 4.42 (s, 1H), 4.22 (t, J=6.7 Hz, 2H), 2.75 (td, J=6.7, 0.9 Hz, 2H), 2.68-2.51 (m, 2H), 2.51-2.42 (m, 2H), 1.76-1.57 (m, 2H), 1.21 (s, 3H).

### 1-[2-(5,6-Difluoroindol-1-yl)ethyl]-3-methyl-pyrrolidin-3-ol

Prepared using general method 3, using 2-(5,6-difluoroindol-1-yl)ethyl methanesulfonate (0.23 g, 0.84 mmol). The title compound was isolated as a viscous oil (114 mg, 48%). Retention time 1.78 mins (10 minute LCMS method); m/z calculated for [C₁₅H₁₅F₂N₂O]⁺ 281.1. Found 281.2 [M+H]⁺; ¹H NMR (400 MHz, d₆-DMSO) δ 7.59 (ddd, J=11.7, 7.0, 0.8 Hz, 1H), 7.50 (dd, J=11.4, 8.1 Hz, 1H), 7.45 (d, J=3.1 Hz, 1H), 6.42 (dd, J=3.1, 0.8 Hz, 1H), 4.43 (s, 1H), 4.21 (t, J=6.5 Hz, 2H), 2.75 (td, J=6.5, 1.2 Hz, 2H), 2.68-2.52 (m, 2H), 2.51-2.42 (m, 2H), 1.76-1.58 (m, 2H), 1.21 (s, 3H).

### General Method 4: Preparation of Fumaric Acid Salt of Pyrrolidine-ol Products

A solution of the pyrrolidinol (0.3 mmol) and fumaric acid (0.3 mmol) in THF (3 mL) was briefly heated to reflux then allowed to cool to rt. The solution was concentrated in vacuo to ca. 1 mL, Et₂O (6 mL) was added and allowed to stand for 2 h. The solvent was decanted and the residue was washed with Et₂O (4 mL) and dried in vacuo to leave the products as a fumarate salt.

### 1-[2-(6-Fluoroindol-1-yl)ethyl]pyrrolidin-3-ol fumarate

Prepared using general method 4. The title compound was isolated as a solid (84 mg). Retention time 1.59 mins (10 minute LCMS method). m/z calculated for [C₁₄H₁₇FN₂O]⁺ 249.1. Found 249.2 [M+H]⁺; ¹H NMR (400 MHz, d₆-DMSO) δ 7.52 (dd, J=8.6, 5.5 Hz, 1H), 7.45-7.30 (m, 2H), 6.86 (ddd, J=9.8, 8.6, 2.3 Hz, 1H), 6.60 (s, 2H), 6.44 (dd, J=3.2, 0.8 Hz, 1H), 4.27 (t, J=6.7 Hz, 2H), 4.24-4.15 (m, 1H), 2.91 (d, J=13.5 Hz, 2H), 2.84 (dd, J=10.1, 5.9 Hz, 1H), 2.75 (dt, J=9.4, 7.5 Hz, 1H), 2.63 (ddd, J=9.3, 8.0, 5.3 Hz, 1H), 2.47 (d, J=3.5 Hz, 1H), 1.96 (dq, J=13.1, 7.4 Hz, 1H), 1.58 (dddd, J=13.0, 8.0, 5.2, 3.2 Hz, 1H).

### 1-[2-(5-Fluoroindol-1-yl)ethyl]pyrrolidin-3-ol fumarate

Prepared using general method 4. The title compound was isolated as a solid (38 mg). Retention time 1.12 min; m/z calculated for [C₁₄H₁₇FN₂O]⁺ 249.1. Found 249.2 [M+H]⁺; ¹H NMR (400 MHz, d₆-DMSO) δ 7.52-7.43 (m, 2H), 7.32-7.25 (m, 1H), 6.96 (td, J=9.2, 2.6 Hz, 1H), 6.61 (s, 1.72H), 6.41 (dd, J=3.1, 0.8 Hz, 1H), 4.28 (t, J=6.8 Hz, 2H), 4.18 (ddt, J=7.1, 6.0, 3.5 Hz, 1H), 3.65-3.56 (m, 1H), 2.87 (d, J=13.5 Hz, 1H), 2.81 (dd, J=9.9, 6.0 Hz, 1H), 2.70 (dt, J=9.0, 7.3 Hz, 1H), 2.58 (ddd, J=9.1, 7.9, 5.3 Hz, 1H), 2.44 (dd, J=10.0, 3.6 Hz, 1H), 2.01-1.88 (m, 1H), 1.82-1.71 (m, 1H), 1.56 (dddd, J=13.0, 8.1, 5.3, 3.3 Hz, 1H).

### 1-[2-(5,6-Difluoroindol-1-yl)ethyl]pyrrolidin-3-ol fumarate

Prepared using general method 4. The title compound was isolated as a solid (50 mg). Retention time 1.73 min (10 minute LCMS method); m/z calculated for [C₁₄H₁₆F₂N₂O]⁺ 267.1. Found 267.1 [M+H]⁺; ¹H NMR (400 MHz, d₆-DMSO) δ 7.61 (ddd, J=11.8, 6.9, 0.8 Hz, 1H), 7.51 (dd, J=11.3, 8.1 Hz, 1H), 7.46 (d, J=3.1 Hz, 1H), 6.61 (s, 1.73H), 6.44 (dd, J=3.2, 0.8 Hz, 1H), 4.27 (t, J=6.7 Hz, 2H), 4.19 (ddt, J=7.0, 5.9, 3.5 Hz, 1H), 2.89 (d, J=13.3 Hz, 2H), 2.82 (dd, J=10.0, 5.9 Hz, 1H), 2.73 (dt, J=9.3, 7.5 Hz, 1H), 2.60 (ddd, J=9.2, 7.9, 5.3 Hz, 1H), 2.46 (dd, J=10.0, 3.6 Hz, 1H), 1.95 (dq, J=13.0, 7.4 Hz, 1H), 1.57 (dddd, J=13.0, 8.1, 5.3, 3.3 Hz, 1H).

### 1-[2-(6-Fluoroindol-1-yl)ethyl]-3-methyl-pyrrolidin-3-ol fumarate

Prepared using general method 4. The title compound was isolated as a solid (34 mg). Retention time 1.16 min; m/z calculated for [C₁₅H₁₉FN₂O]⁺ 263.1. Found 263.2 [M+H]⁺; ¹H NMR (400 MHz, CDCl₃) δ 7.53-7.49 (m, 1H), 7.15 (d, J=3.2 Hz, 1H), 7.07 (d, J=9.6 Hz, 1H), 6.89-6.83 (m, 1H), 6.82 (s, 1.8H), 6.47 (t, J=3.0 Hz, 1H), 4.41 (t, J=3.6 Hz, 2H), 3.27-3.21 (m, 3H), 3.04 (d, J=10.4 Hz, 1H), 2.81 (d, J=4.4 Hz, 1H), 2.71 (dd, J=10.8, 2.8 Hz, 1H), 1.98-1.95 (m, 2H), 1.85 (d, J=3.6 Hz, 1H), 1.36 (d, J=3.6 Hz, 3H).

### 1-[2-(5-Fluoroindol-1-yl)ethyl]-3-methyl-pyrrolidin-3-ol fumarate

Prepared using general method 4. The title compound was isolated as a solid (19 mg). Retention time 1.14 min; m/z calculated for [C₁₅H₁₉FN₂O]⁺ 263.1. Found 263.2 [M+H]⁺; ¹H NMR (400 MHz, d₆-DMSO) δ 7.52-7.42 (m, 2H), 7.29 (dd, J=9.9, 2.5 Hz, 1H), 6.96 (td, J=9.2, 2.5 Hz, 1H), 6.60 (s, 1.85H), 6.41 (dd, J=3.1, 0.8 Hz, 1H), 4.28 (t, J=6.8 Hz, 2H), 3.65-3.55 (m, 1H), 2.89 (d, J=13.5 Hz, 2H), 2.82-2.64 (m, 2H), 2.59 (d, J=1.7 Hz, 2H), 1.82-1.63 (m, 2H), 1.22 (s, 3H).

### 1-[2-(5,6-Difluoroindol-1-yl)ethyl]-3-methyl-pyrrolidin-3-ol fumarate

Prepared using general method 4. The title compound was isolated as a solid (75 mg). Retention time 1.87 min (10 minute LCMS method); m/z calculated for [C₁₅H₁₈F₂N₂O]⁺ 281.1. Found 281.2 [M+H]⁺; ¹H NMR (400 MHz, d₆-DMSO) δ 7.60 (ddd, J=11.7, 7.0, 0.8 Hz, 1H), 7.50 (dd, J=11.3, 8.1 Hz, 1H), 7.45 (d, J=3.1 Hz, 1H), 6.60 (s, 2H), 6.43 (dd, J=3.2, 0.8 Hz, 1H), 4.26 (t, J=6.7 Hz, 2H), 3.65-3.56 (m, 1H), 2.89 (d, J=13.3 Hz, 2H), 2.82-2.66 (m, 2H), 2.65-2.55 (m, 2H), 1.82-1.63 (m, 2H), 1.22 (s, 3H).

### General method 5: Displacement with 2-(methylamino)ethanol

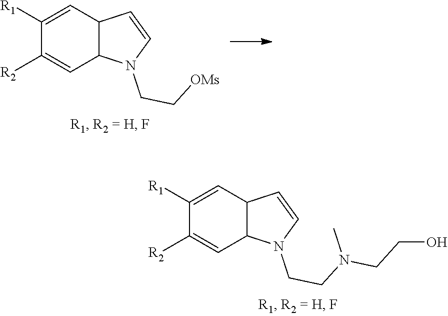

A suspension of the mesylate (2 mmol), K₂CO₃ (2.1 mmol) and 2-(methylamino)ethanol (3 mmol) in acetonitrile (6 mL) was heated to 60° C. (oil bath temperature) and stirred for 20 h. The reaction mixture was cooled to rt, H₂O (20 mL) was added and the mixture extracted with DCM (2×14 mL). The combined organic layers were washed with brine (14 mL), concentrated in vacuo and purified by column chromatography on silica gel (eluent: 5% MeOH in DCM) to give the product.

### 2-[2-(6-Fluoroindol-1-yl)ethyl-methyl-amino]ethanol

Prepared using general method 5, using 2-(6-fluoroindol-1-yl)ethyl methanesulfonate (0.385 g, 1.50 mmol). The title compound was isolated as a viscous oil (121 mg, 34%). Retention time 1.12 min; m/z calculated for [C₁₃H₁₇FN₂O]⁺ 237.1. Found 237.2 [M+H]⁺; ¹H NMR (400 MHz, d₆-DMSO) δ 7.51 (dd, J=8.6, 5.5 Hz, 1H), 7.38 (d, J=3.2 Hz, 1H), 7.32 (ddt, J=10.6, 2.4, 0.7 Hz, 1H), 6.85 (ddd, J=9.8, 8.6, 2.4 Hz, 1H), 6.42 (dd, J=3.1, 0.9 Hz, 1H), 4.24-4.16 (m, 3H), 3.40 (td, J=6.3, 5.3 Hz, 2H), 2.72 (t, J=6.6 Hz, 2H), 2.46 (t, J=6.2 Hz, 2H), 2.24 (s, 3H).

### 2-[2-(5-Fluoroindol-1-yl)ethyl-methyl-amino]ethanol

Prepared using general method 5, using 2-(5-fluoroindol-1-yl)ethyl methanesulfonate (0.60 g, 2.33 mmol). The title compound was isolated as a viscous oil (206 mg, 37%). Retention time 1.52 min (10 minute LCMS method); m/z calculated for [C₁₃H₁₇FN₂O]⁺ 237.1. Found 237.2 [M+H]⁺; ¹H NMR (400 MHz, d₆-DMSO) δ 7.51-7.43 (m, 2H), 7.27 (ddd, J=9.9, 2.6, 0.5 Hz, 1H), 6.95 (td, J=9.2, 2.6 Hz, 1H), 6.39 (dd, J=3.1, 0.8 Hz, 1H), 4.27-4.17 (m, 3H), 3.39 (td, J=6.2, 5.2 Hz, 2H), 2.72 (t, J=6.6 Hz, 2H), 2.45 (t, J=6.3 Hz, 2H), 2.24 (s, 3H).

### 2-[2-(5,6-Difluoroindol-1-yl)ethyl-methyl-amino]ethanol

Prepared using general method 5, using 2-(5,6-difluoroindol-1-yl)ethyl methanesulfonate (0.40 g, 1.45 mmol). The title compound was isolated as a viscous oil (93 mg, 25%). Retention time 1.69 min (10 minute LCMS method); m/z calculated for [C₁₃H₁₆F₂N₂O]⁺ 255.1; found 255.0 [M+H]⁺; ¹H NMR (400 MHz, d₆-DMSO) δ 7.59 (ddd, J=11.6, 6.9, 0.8 Hz, 1H), 7.54-7.47 (m, 1H), 7.47-7.42 (m, 1H), 6.41 (dd, J=3.1, 0.8 Hz, 1H), 4.24-4.16 (m, 3H), 3.38 (td, J=6.2, 5.3 Hz, 2H), 2.71 (t, J=6.6 Hz, 2H), 2.45 (t, J=6.2 Hz, 2H), 2.23 (s, 3H).

### General Method 6: Preparation of Fumaric Acid Salt of Methylethanolamine Product

A solution of the above methylethanolamine (0.3 mmol) and fumaric acid (0.3 mmol) in THF (3 mL) was briefly heated to reflux then allowed to cool to rt. The solution was concentrated to ca. 1 mL, Et₂O (6 mL) was added and allowed to stand for 2 h. The solvent was decanted, the residue washed with Et₂O (4 mL) and dried in vacuo to give the product.

### 2-[2-(6-Fluoroindol-1-yl)ethyl-methyl-amino]ethanol fumarate

Prepared using general method 6. The title compound was isolated as a solid (39 mg). Retention time 1.13 min; m/z calculated for [C₁₃H₁₇FN₂O]⁺ 237.1. Found 237.2 [M+H]⁺; ¹H NMR (400 MHz, d₆-DMSO) δ 7.51 (dd, J=8.6, 5.5 Hz, 1H), 7.38 (d, J=3.2 Hz, 1H), 7.34 (ddt, J=10.6, 2.4, 0.6 Hz, 1H), 6.85 (ddd, J=9.8, 8.6, 2.4 Hz, 1H), 6.61 (s, 2H), 6.43 (dd, J=3.2, 0.9 Hz, 1H), 4.25 (t, J=6.7 Hz, 2H), 3.44 (t, J=6.1 Hz, 2H), 2.82 (t, J=6.8 Hz, 2H), 2.56 (t, J=6.1 Hz, 2H), 2.32 (s, 3H).

### 2-[2-(5-Fluoroindol-1-yl)ethyl-methyl-amino]ethanol fumarate

Prepared using general method 6. The title compound was isolated as a solid (58 mg). Retention time 1.59 min (10 minute LCMS method); m/z calculated for [C₁₃H₁₇FN₂O]⁺ 237.1. Found 237.2 [M+H]⁺; ¹H NMR (400 MHz, d₆-DMSO) δ 7.52-7.43 (m, 2H), 7.32-7.24 (m, 1H), 6.96 (td, J=9.2, 2.5 Hz, 1H), 6.62 (s, 2H), 6.40 (dd, J=3.1, 0.8 Hz, 1H), 4.26 (t, J=6.7 Hz, 2H), 3.43 (d, J=6.2 Hz, 2H), 2.79 (t, J=6.7 Hz, 2H), 2.53 (d, J=6.2 Hz, 2H), 2.29 (s, 3H).

### 2-[2-(5,6-Difluoroindol-1-yl)ethyl-methyl-amino]ethanol fumarate

Prepared using general method 6. The title compound was isolated as a solid (41 mg). Retention time 1.72 min (10 minute LCMS method); m/z calculated for [C₁₃H₁₆F₂N₂O]⁺ 255.1; found 255.2 [M+H]⁺; ¹H NMR (400 MHz, d₆-DMSO) δ 7.60 (ddd, J=11.7, 7.0, 0.8 Hz, 1H), 7.54-7.47 (m, 1H), 7.47-7.43 (m, 1H), 6.61 (s, 2H), 6.42 (dd, J=3.1, 0.9 Hz, 1H), 4.24 (t, J=6.6 Hz, 2H), 3.41 (d, J=6.1 Hz, 2H), 2.79 (t, J=6.6 Hz, 2H), 2.56-2.50 (m, 2H), 2.29 (s, 3H).

### General Method 7: Displacement with N-Methyl-2-Methylsulfonyl-Ethanamine

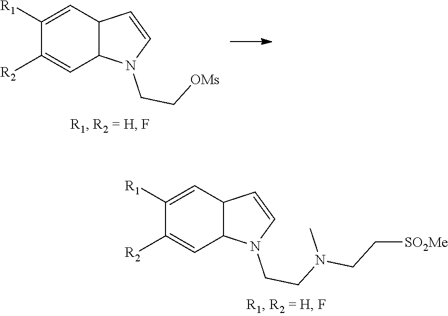

A suspension of the mesylate (2 mmol), K₂CO₃ (2.1 mmol) and N-methyl-2-methylsulfonyl-ethanamine (3 mmol) in acetonitrile (6 mL) was heated to 80° C. (oil bath temperature) and stirred for 96 h. The mixture was cooled to rt, H₂O (20 mL) added and extracted with DCM (2×14 mL). The combined organic layers were washed with brine (14 mL), concentrated in vacuo and purified by column chromatography on silica gel (eluent: 0 to 3% MeOH in DCM). The products required further purification by reverse-phase preparative HPLC (eluent: 19-29% MeCN in 0.1% aq formic acid gradient). Pure fractions were combined and lyophilised. To remove excess formic acid, The products were dissolved in DCM (6 mL), washed with diluted NaHCO₃ solution (3 mL), dried over MgSO₄, filtered and concentrated in vacuo.

### 2-(6-Fluoroindol-1-yl)-N-methyl-N-(2-methylsulfonylethyl)ethanamine

Prepared using general method 7, using 2-(6-fluoroindol-1-yl)ethyl methanesulfonate (0.335 g, 1.30 mmol). The title compound was isolated as a viscous oil (82 mg, 21%). Retention time 1.89 min (10 minute LCMS method); m/z calculated for [C₁₄H₁₉FN₂O₂S]⁺ 299.1. Found 299.0 [M+H]⁺; ¹H NMR (400 MHz, d₆-DMSO) δ 7.51 (dd, J=8.6, 5.5 Hz, 1H), 7.44-7.29 (m, 2H), 6.85 (ddd, J=9.8, 8.6, 2.4 Hz, 1H), 6.43 (dd, J=3.2, 0.9 Hz, 1H), 4.22 (t, J=6.6 Hz, 2H), 3.32-3.10 (m, 2H), 2.86-2.62 (m, 7H), 2.26 (s, 3H).

### 2-(5-Fluoroindol-1-yl)-N-methyl-N-(2-methylsulfonylethyl)ethanamine

Prepared using general method 7, using 2-(5-fluoroindol-1-yl)ethyl methanesulfonate (0.40 g, 1.55 mmol). The title compound was isolated as a viscous oil (53 mg, 11%). Retention time 1.85 min (10 minute LCMS method); m/z calculated for [C₁₄H₁₉FN₂O₂S]⁺ 299.1. Found 299.0 [M+H]⁺; ¹H NMR (400 MHz, d₆-DMSO) δ 7.55-7.41 (m, 2H), 7.28 (dd, J=9.9, 2.4 Hz, 1H), 6.96 (td, J=9.2, 2.5 Hz, 1H), 6.40 (dd, J=3.1, 0.8 Hz, 1H), 4.26 (t, J=6.6 Hz, 2H), 3.33-3.10 (m, 2H), 2.87-2.60 (m, 7H), 2.25 (s, 3H).

### 2-(5,6-Difluoroindol-1-yl)-N-methyl-N-(2-methylsulfonylethyl)ethanamine

Prepared using general method 7, using 2-(5,6-difluoroindol-1-yl)ethyl methanesulfonate (0.40 g, 1.45 mmol). The title compound was isolated as a viscous oil (79 mg, 17%). Retention time 2.09 min (10 minute LCMS method); m/z calculated for [C₁₃H₁₆F₂N₂O]⁺ 317.1. Found 317.0 [M+H]⁺; ¹H NMR (400 MHz, d₆-DMSO) δ 7.62 (ddd, J=11.8, 7.0, 0.9 Hz, 1H), 7.54-7.42 (m, 2H), 6.42 (dd, J=3.1, 0.8 Hz, 1H), 4.23 (t, J=6.5 Hz, 2H), 3.32-3.10 (m, 2H), 2.86-2.59 (m, 7H), 2.25 (s, 3H).

### General Method 8: Preparation of Fumaric Acid Salt of N-Methyl-2-Methylsulfonyl-Ethanamine Product

A solution of the above N-methyl-2-methylsulfonyl-ethanamine (0.3 mmol) and fumaric acid (0.3 mmol) in THF (3 mL) was briefly heated to reflux then allowed to cool to rt. The solution was concentrated in vacuo to ca. 1 mL, Et₂O (6 mL) added and allowed to stand for 2 h. The solvent was decanted and the residue washed with Et₂O (4 mL) and dried in vacuo.

### 2-(5-Fluoroindol-1-yl)-N-methyl-N-(2-methylsulfonylethyl)ethanamine fumarate

Prepared using general method 8. The title compound was isolated as a solid (12 mg). Retention time 1.88 min (10 minute LCMS method); m/z calculated for [C₁₄H₁₉FN₂O₂S]⁺ 299.1. Found 299.0 [M+H]⁺; ¹H NMR (400 MHz, d₆-DMSO) δ 7.55-7.41 (m, 2H), 7.28 (dd, J=10.0, 2.6 Hz, 1H), 6.96 (td, J=9.2, 2.6 Hz, 1H), 6.63 (s, 2H), 6.40 (dd, J=3.2, 0.8 Hz, 1H), 4.26 (t, J=6.6 Hz, 2H), 3.17 (t, J=6.8 Hz, 2H), 2.87-2.67 (m, 7H), 2.25 (s, 3H).

### 2-(5,6-Difluoroindol-1-yl)-N-methyl-N-(2-methylsulfonylethyl)ethanamine fumarate

Prepared using general method 8. The title compound was isolated as a solid (17 mg). Retention time 2.11 min (10 minute LCMS method); m/z calculated for [C₁₃H₁₆F₂N₂O]⁺ 317.1. Found 317.0 [M+H]⁺; ¹H NMR (400 MHz, d₆-DMSO) δ 7.62 (ddd, J=11.7, 7.0, 0.9 Hz, 1H), 7.55-7.41 (m, 2H), 6.63 (s, 1H), 6.42 (dd, J=3.2, 0.8 Hz, 1H), 4.23 (t, J=6.5 Hz, 2H), 3.23-3.09 (m, 2H), 2.86-2.59 (m, 7H), 2.25 (s, 3H).

### 2-(5-Fluoro-1H-indol-3-yl)-N-(2-hydroxyethyl)-N-methyl-2-oxo-acetamide

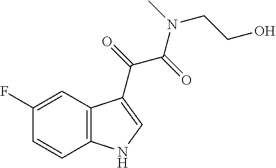 [SMILES: CN(CCO)C(=O)C(=O)c1c[nH]c2ccc(F)cc12]

To a solution of 2-(methylamino)ethanol (0.375 g, 4.99 mmol) in DCM (5 mL) was added at 0° C. N,N-diisopropylethylamine (1.14 mL, 6.65 mmol) followed by 2-(5-fluoro-1H-indol-3-yl)-2-oxoacetyl chloride [CAS No: 3828-09-9](0.75 g, 3.32 mmol) in THF (7.5 mL). The reaction mixture was stirred at 15° C. for 2 h. On completion, aq. NaCl (30 mL) was added, and the mixture was stirred for 5 min. The aqueous phase was extracted with DCM (20 mL×3). The combined organic layers were dried (Na₂SO₄), filtered, and concentrated in vacuo. The residue was purified by column chromatography on silica gel (eluent: 1-10% MeOH in EtOAc gradient) to afford the title compound (574 mg, 65%) as a solid. Retention time: 1.28 min; m/z calculated for [C₁₃H₁₃FN₂O₃]⁺ 265.25. found 265.25 [M+H]⁺; ¹H NMR (DMSO-d₆, 400 MHz, 90° C.) δ 12.10 (s, 1H), 8.08 (d, J=3.2 Hz, 1H), 7.77 (td, J=9.0, 2.3 Hz, 1H), 7.56-7.51 (m, 1H), 7.12-7.07 (m, 1H), 4.54 (m, 0.5H, conformer), 4.33 (m, 0.5H, conformer), 3.71-3.67 (m, 1H), 3.10 (s, 3H). Note: ¹H NMR shows two conformers; When heated at 90° C. the ¹H NMR shows only one peak for each proton, only a couple of peaks remained split.

### 2-[2-(5-Fluoro-1H-indol-3-yl)ethyl-methyl-amino]ethanol

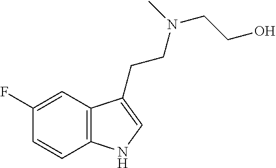 [SMILES: CN(CCO)CCc1c[nH]c2ccc(F)cc12]

LiAlH₄ (314 mg, 8.26 mmol) was taken up in anhydrous THF (10 mL) under an atmosphere of N₂ was added portion-wise 2-(5-fluoro-1H-indol-3-yl)-N-(2-hydroxyethyl)-N-methyl-2-oxo-acetamide (0.364 g, 1.38 mmol) with stirring at 0° C. The reaction mixture was heated to reflux (65° C.) and stirred for 6 h. LCMS showed that the reaction was not complete, so the reaction mixture was cooled to 0° C. and 80 mg (1.5 eq) of LiAlH₄ were added to the mixture. The reaction was then heated to reflux and stirred for an additional 4 h. After cooling to 10° C. the mixture was poured dropwise onto ice-cold H₂O (10 mL, NB: The quenching of the reaction mixture was exothermic in nature). To the aqueous suspension was added EtOAc (50 mL) and both layers were stirred well. Both layers were filtered through a pad of celite and the solids washed on the filter with EtOAc (100 mL). The organic layer was separated, washed with brine (2×30 mL) dried over Na₂SO₄, filtered, and the filtrate was removed under reduced pressure at 50° C. The residue was purified by column chromatography on silica gel (eluent: 1-10% MeOH in EtOAc gradient) to afford the title compound (168 mg, 30%) as a solid. Retention time: 1.05 min; m/z calculated for [C₁₃H₁₇FN₂O]⁺ 237.0. found 237.0 [M+H]⁺; ¹H NMR (CDCl₃, 400 MHz) δ 7.98 (br. s, 1H), 7.27-7.20 (m, 2H), 7.06 (d, J=2.4 Hz, 1H), 6.93 (td, J=9.0 2.0 Hz, 1H), 3.55 (t, J=6.0 Hz, 2H), 2.87 (d, J=8.0 Hz, 2H), 2.76 (t, J=6.0 Hz, 2H), 2.60 (t, J=6.0 Hz, 2H), 2.37 (s, 3H).

### 2-[2-(5-Fluoro-1H-indol-3-yl)ethyl-methyl-amino]ethanol fumarate salt

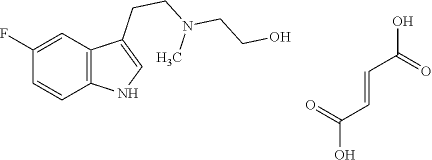 [SMILES: CN(CCO)CCc1c[nH]c2ccc(F)cc12 ; O=C(O)/C=C/C(=O)O]

To solution of 2-[2-(5-fluoro-1H-indol-3-yl)ethyl-methyl-amino]ethanol (88 mg, 0.372 mmol) in THF (2 mL) was added at 0° C. fumaric acid (43 mg, 0.372 mmol) dissolved in THF (2 mL). The mixture was stirred for 1 h, after which time the salt started to precipitate. The solid was then filtered, rinsed with Et₂O (2×2 mL), sonicated in Et₂O (1 mL) and filtered. The solid was rinsed with Et₂O and dried under vacuum overnight (no heating) to give the fumarate salt (66 mg, 67%) as a solid. Retention time: 1.32 min; m/z calculated for [C₁₃H₁₇FN₂O]⁺ 237.0. found 237.0 [M+H]⁺; ¹H NMR (DMSO-d₆, 400 MHz) δ 10.87 (br. s, 1H), 7.33-7.22 (m, 3H), 6.89-6.88 (m, 1H), 6.54 (s, 1H, fumarate), 3.55 (t, J=6.0 Hz, 2H), 2.87-2.76 (m, 4H), 2.65 (t, J=6.0 Hz, 2H), 2.41 (s, 3H). Traces of THF: 5.25 mol %.

### 1-(5-fluoro-1H-indol-3-yl)-2-(3-hydroxypyrrolidin-1-yl)ethane-1,2-dione

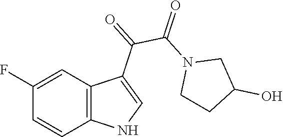 [SMILES: O=C(C(=O)N1CCC(O)C1)c1c[nH]c2ccc(F)cc12]

To a solution of pyrrolidin-3-ol (0.30 g, 1.33 mmol) in DCM (5 mL) at 0° C. was added N,N-diisopropylethylamine (0.455 mL g, 2.66 mmol) followed by 2-(5-fluoro-1H-indol-3-yl)-2-oxoacetyl chloride (0.30 g, 1.33 mmol) dissolved in THF (10 mL). The mixture was warmed to 15° C. and stirred for 2 h. On completion, aq. NaCl (30 mL) was added, and the mixture was stirred for 5 min. The aqueous phase was extracted with DCM (50 mL×3) and the combined organic layers were dried (Na₂SO₄), filtered, and concentrated in vacuo. The residue was purified by column chromatography on silica gel (eluent: 1-10% MeOH in DCM gradient). Pure fractions were combined and concentrated in vacuo, and the residue was triturated with Et₂O (3×10 mL) and the precipitate was isolated by filtration to afford the title compound (186 mg, 50%) as a solid. Retention time: 1.30 min; m/z calculated for [C₁₄H₁₃FN₂O₃]⁺ 277.2. found 277.0 [M+H]⁺; ¹H NMR (DMSO-d₆, 400 MHz) shows the presence of two conformers δ 12.32 (br. s, 1H), 8.21 (d, J=8.4 Hz. 1H), 7.79 (dd, J=9.6 Hz, 2.4 Hz, 1H), 7.54 (dd, J=8.8, 4.4 Hz, 1H), 7.13 (td, J=9.0, 2.0 Hz, 1H), 6.99 (d, J=3.8 Hz, 0.4H), 4.94 (d, J=3.6 Hz, 0.6 Hz), 4.34 (s, 0.4H). 4.28 (s, 0.6H), 3.58-3.40 (m, 1H), 1.91-1.83 (m, 1H).

### 1-[2-(5-fluoro-1H-indol-3-yl)ethyl]pyrrolidin-3-olethanamine

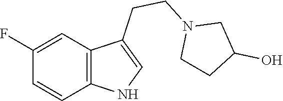 [SMILES: OC1CCN(CCc2c[nH]c3ccc(F)cc23)C1]

Lithium aluminium hydride (0.184 g, 4.84 mmol) was taken in anhydrous THF (10 mL) at 0° C. under an atmosphere of N₂ and 1-(5-fluoro-1H-indol-3-yl)-2-(3-hydroxypyrrolidin-1-yl)ethane-1,2-dione (0.223 g, 0.807 mmol) was added portion-wise with stirring. The mixture was heated to reflux (65° C.) and stirred for 8 h, then cooled to 10° C. poured onto ice-cold water (10 mL) drop-wise (The quenching of the reaction was exothermic). To the obtained suspension was added EtOAc (50 mL) and both layers were stirred well, filtered through a pad of celite and filter cake washed with EtOAc (100 mL). The layers were separated, and the organic layer washed with brine (2×30 mL), dried over MgSO₄, filtered and the filtrate was removed under reduced pressure at 50° C. The crude product was purified by column chromatography on silica gel (eluent: 5% 7N NH₃ in MeOH/EtOAc) to afford the title compound (98 mg, 48%) as a solid. Retention time: 1.07 min; m/z calculated for [C₁₄H₁₇FN₂O]⁺ 249.2. found 249.2 [M+H]⁺; ¹H NMR (400 MHz, DMSO-d₆) δ 10.81 (br. s, 1H), 7.30 (dd, J=8.8, 4.4 Hz, 1H), 7.24-7.20 (m, 2H), 6.87 (td, J=9.2, 2.4 Hz, 1H), 4.59 (d, J=4.4 Hz, 1H), 4.21-4.17 (m, 1H), 2.80-2.74 (m, 2H), 2.66-2.60 (m, 2H), 2.48-2.46 (m, 2H), 2.37 (dd, J=9.6, 4.0 Hz), 2.02-1.93 (m, 1H), 1.80-1.51 (m, 1H).

### 1-[2-(5-fluoro-1H-indol-3-yl)ethyl]pyrrolidin-3-olethanamine fumarate salt

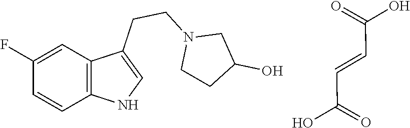 [SMILES: OC1CCN(CCc2c[nH]c3ccc(F)cc23)C1 ; O=C(O)/C=C/C(=O)O]

To a mixture of 1-[2-(5-fluoro-1H-indol-3-yl)ethyl]pyrrolidin-3-olethanamine (50 mg, 0.20 mmol) dissolved in THF (2 mL) at 0° C. was added fumaric acid (23.3 mg, 0.20 mmol) dissolved in THF (2 mL). After stirring for 1 h, the salt started to precipitate. The solid was filtered, rinsed with Et₂O (2×2 mL), then sonicated in Et₂O (1 mL) and filtered. The solid was rinsed with Et₂O and dried in a vacuum oven overnight (no heating) to afford the title compound (58 mg, 79%) as a solid. Retention time: 1.40 min; ¹H NMR (DMSO-d₆, 400 MHz,) δ 10.87 (s, 1H), 7.32 (dd, J=8.8, 4.6 Hz, 1H), 7.27 (dd, J=10.1, 2.6 Hz, 1H), 7.24 (d, J=2.5 Hz, 1H), 6.89 (td, J=9.2, 2.5 Hz, 1H), 6.52 (s, 1H), 4.25 (dq, J=9.3, 3.4 Hz, 1H), 3.64-3.57 (m, 1H, THF), 2.92 (dd, J=10.2, 5.9 Hz, 1H), 2.88-2.77 (m, 5H), 2.76-2.66 (m, 1H), 2.60 (dd, J=10.2, 3.4 Hz, 1H), 2.02 (dq, J=13.3, 7.3 Hz, 1H), 1.82-1.71 (m, 2H), 1.69-1.57 (m, 1H, THF). Trace of THF: 29.2 mol %.

### 1-(5-fluoro-1H-indol-3-yl)-2-(3-hydroxy-3-methyl-pyrrolidin-1-yl)ethane-1,2-dione

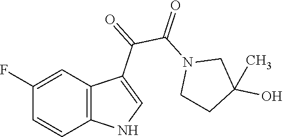 [SMILES: CC1(O)CCN(C(=O)C(=O)c2c[nH]c3ccc(F)cc23)C1]

To a mixture of 3-methylpyrrolidin-3-ol (0.28 g, 2.77 mmol) in DCM (5 mL) at 0° C. was added N,N-diisopropylethylamine (0.76 mL g, 4.43 mmol) and 2-(5-fluoro-1H-indol-3-yl)-2-oxoacetyl chloride (0.50 g, 2.22 mmol) in THF (10 mL). The mixture was warmed to 15° C. and stirred for 2 h, then aq. NaCl (30 mL) was added, and the mixture was stirred for 5 min. The mixture was extracted with DCM (50 mL×3) and the combined organic layers were dried (Na₂SO₄), filtered, and concentrated in vacuo. The crude product was purified by column chromatography on silica gel (eluent: 1-10% MeOH in DCM) to afford the title compound (330 mg, 51%) as a solid. Retention time: 1.34 min; m/z calculated for [C₁₅H₁₅FN₂O₃]⁺ 291.0. found 291.2 [M+H]⁺; ¹H NMR (DMSO-d₆ 400 MHz,) δ 12.34 (s, 1H), 8.21 (d, J=11.3 Hz, 1H), 7.80 (dd, J=9.7, 2.6 Hz, 1H), 7.55 (ddd, J=8.8, 4.6, 0.7 Hz, 1H), 7.13 (td, J=9.2, 2.6 Hz, 1H), 4.82 (split singlet due to conformers, 1H), 3.50-3.48 (m, 3H), 3.39-3.20 (m, 2H), 1.91-1.75 (m, 2H), 1.24 (s, 3H).

### 1-[2-(5-fluoro-1H-indol-3-yl)ethyl]-3-methyl-pyrrolidin-3-ol

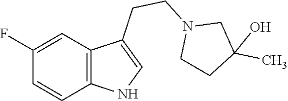 [SMILES: CC1(O)CCN(CCc2c[nH]c3ccc(F)cc23)C1]

LiAlH₄ (0.216 g, 5.68 mmol) was taken in anhydrous THF (5 mL) at 0° C. under an atmosphere of N₂ was added 1-(5-fluoro-1H-indol-3-yl)-2-(3-hydroxy-3-methyl-pyrrolidin-1-yl)ethane-1,2-dione (0.33 g, 1.14 mmol) portion-wise with stirring. The mixture was heated to reflux and stirred for 5 h. LCMS shows that the reaction was not entirely complete, so the mixture was cooled to 0° C., and LiAlH₄ (120 mg, 3 eq) was added. The mixture was refluxed for a further 4 h then cooled to 10° C. and was then poured drop-wise onto ice-cold water (10 mL) [note: quenching was exothermic]. To the suspension obtained was added EtOAc (50 mL) and both layers were stirred well, filtered through a pad of celite and the filter cake was washed with EtOAc (100 mL). The layers were separated, and the organic layer washed with brine (2×30 mL), dried over Na₂SO₄, filtered and the solvent was removed in vacuo at 50° C. The crude product was purified by column chromatography on silica gel (eluent: 5% 7N NH₃ in MeOH/EtOAc) to afford the title compound (220 mg, 73%) as a solid. Retention time: 1.11 min; m/z calculated for [C₁₅H₁₉FN₂O]⁺ 263.3. found 263.2 [M+H]⁺; ¹H NMR (CDCl₃, 400 MHz,) δ 7.98 (s, 1H), 7.29-7.20 (m, 2H), 7.07 (d, J=2.4 Hz, 1H), 6.93 (td, J=9.0 Hz, 2.6 Hz, 1H), 3.08 (ddd, J=9.2, 7.6, 6.3 Hz, 1H), 2.95-2.86 (m, 2H), 2.85 (dt, J=9.4, 0.9 Hz, 1H), 2.83-2.73 (m, 2H), 2.43-2.33 (m, 1H), 2.30 (d, J=9.3 Hz, 1H), 1.97-1.83 (m, 2H), 1.38 (s, 3H).

### 1-[2-(5-fluoro-1H-indol-3-yl)ethyl]-3-methyl-pyrrolidin-3-ol fumarate salt

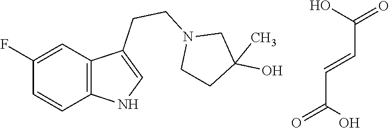 [SMILES: CC1(O)CCN(CCc2c[nH]c3ccc(F)cc23)C1 ; O=C(O)/C=C/C(=O)O]

To a mixture of 1-[2-(5-fluoro-1H-indol-3-yl)ethyl]-3-methyl-pyrrolidin-3-ol (65 mg, 0.24 mmol) in THF (2 mL) was added a solution of fumaric acid (28 mg, 0.24 mmol) in THF (2 mL). At the beginning of the addition of the solution of fumaric acid the solution of the free base turned cloudy but then fully dissolved. The solvent was evaporated and the resulting foamy solid was dried under vacuum. The solid residue was triturated with Et₂O and filtered, and the solid was rinsed with Et₂O and dried under vacuum overnight (no heating) to give the fumarate salt (58 mg, 62%) as a solid. Retention time: 1.57 min; m/z calculated for [C₁₅H₁₉FN₂O]⁺ 263.3. found 263.2 [M+H]⁺; ¹H NMR (DMSO-d₆, 400 MHz,) δ 10.92 (s, 1H), 7.37-7.27 (m, 2H), 7.25 (d, J=2.5 Hz, 1H), 6.90 (td, J=9.2, 2.6 Hz, 1H), 6.54 (s, 2H), 3.14-3.03 (m, 1H), 3.03-2.93 (m, 3H), 2.96-2.86 (m, 3H), 2.85 (d, J=10.3 Hz, 1H), 1.90-1.80 (m, 2H), 1.30 (s, 3H).

### 2-(5-fluoro-1H-indol-3-yl)ethyl methanesulfonate

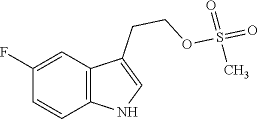 [SMILES: CS(=O)(=O)OCCc1c[nH]c2ccc(F)cc12]

To a mixture of 2-(5-fluoro-1H-indol-3-yl)ethanol [CAS No: 101349-12-6](150 mg, 0.837 mmol) in DCM (5 mL) at 0° C. was added Et₃N (0.14 mL, 1.0 mmol) followed by dropwise addition of methanesulfonyl chloride (0.077 mL, 1.0 mmol). The mixture was warmed to rt and stirred for 3 h, then H₂O and DCM was added. The mixture was partitioned and the organic layer was washed with brine (5 mL), dried over MgSO₄, filtered, and concentrated to give the crude product (274 mg, >100%). This material was used in the next step with no further purification. ¹H NMR (CDCl₃, 400 MHz) δ 8.07 (br. s, 1H), 7.30-7.21 (m, 3H), 7.15 (s, 1H), 6.95 (td, J=9.2, 1.6 Hz, 1H), 4.45 (t, J=6.0 Hz, 2H), 3.19-3.16 (m, 2H), 2.89 (s, 3H).

### 2-(5-fluoro-1H-indol-3-yl)-N-methyl-N-(2-methylsulfonylethyl)ethanamine

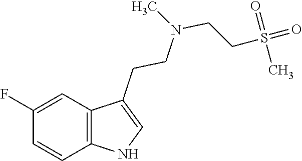 [SMILES: CN(CCc1c[nH]c2ccc(F)cc12)CCS(C)(=O)=O]

To a mixture of 2-(5-fluoro-1H-indol-3-yl)ethyl methanesulfonate (0.270 g, 1.05 mmol) in acetonitrile (10 mL) was added N-methyl-2-methylsulfonyl-ethanamine (0.173 g, 1.26 mmol) followed by K₂CO₃ (0.29 g, 2.10 mmol). The mixture was heated to 65° C. and stirred for 3 h, then evaporated to dryness and the residue taken up in DCM (25 mL). The mixture was washed with brine (10 mL), dried over MgSO₄, filtered and the filtrate was concentrated in vacuo. The crude product was purified twice by column chromatography on silica gel (eluent: 1-10% MeOH in EtOAc) to afford the title compound as (55 mg, 17%) as a viscous oil. Retention time: 1.10 min; m/z calculated for [C₁₄H₁₉FN₂O₂S]⁺ 299.3. found 299.2 [M+H]⁺; ¹H NMR (CDCl₃, 400 MHz) δ 7.99 (br. s, 1H), 7.28-7.23 (m, 1H), 7.20 (dd, J=9.4, 2.4 Hz, 1H), 7.07 (d, J=2.4 Hz, 1H), 6.93 (td, J=9.4, 2.4 Hz, 1H), 3.07 (t, J=6.0 Hz, 2H), 2.95 (t, J=6.0 Hz, 2H), 2.89 (t, J=7.6 Hz, 2H), 2.80 (s, 3H), 2.72 (t, J=7.2 Hz, 2H), 2.35 (s, 3H).

### 2-(5-fluoro-1H-indol-3-yl)-N-methyl-N-(2-methylsulfonylethyl)ethanamine fumarate salt

To a mixture of 2-(5-fluoro-1H-indol-3-yl)-N-methyl-N-(2-methylsulfonylethyl)ethanamine (22 mg, 0.0737 mmol) in dry THF (1 mL) was added fumaric acid (8.55 mg, 0.0737 mmol) dissolved in dry THF (1 mL). The solid that precipitated was filtered and rinsed with Et₂O (2×2 mL) and dried under vacuum to give the fumarate salt (12 mg, 39%) as a solid. Retention time: 1.49 min; m/z calculated for [C₁₄H₁₉FN₂O₂S]⁺ 299.3. found 299.2 [M+H]⁺; ¹H NMR (DMSO-d₆, 400 MHz,) δ 10.84 (s, 1H), 7.31 (dd, J=8.8, 4.6 Hz, 1H), 7.30-7.21 (m, 2H), 6.88 (td, J=9.2, 2.5 Hz, 1H), 6.62 (s, 2H, fumarate salt), 3.26 (t, J=6.9 Hz, 2H), 2.93 (s, 3H), 2.86-2.78 (m, 4H), 2.70-2.62 (m, 2H), 2.30 (s, 3H).

### 1-(6-fluoro-1H-indol-3-yl)-2-(3-hydroxy-3-methyl-pyrrolidin-1-yl)ethane-1,2-dione

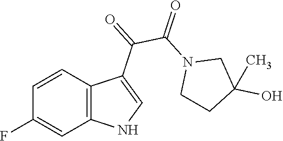 [SMILES: CC1(O)CCN(C(=O)C(=O)c2c[nH]c3cc(F)ccc23)C1]

To a mixture of 3-methylpyrrolidin-3-ol (0.28 g, 2.77 mmol, 1.25 eq) in DCM (5 mL) at 0° C. was added N,N-diisopropylethylamine (0.759 mL g, 4.43 mmol, 2 eq) followed by 2-(5-fluoro-1H-indol-3-yl)-2-oxoacetyl chloride (0.500 g, 2.22 mmol, 1 eq) in THF (10 mL). The mixture was warmed to 15° C. and stirred for 2 h, then the solvent was evaporated, and the residue triturated with H₂O. The solid was filtered, rinsed with H₂O and then with Et₂O. The solid was dried under vacuum overnight to afford the title compound (551 mg, 85%) as a solid. Retention time: 1.35 min; m/z calculated for [C₁₅H₁₅FN₂O₃]⁺ 291.0 found 291.0 [M+H]⁺; ¹H NMR (DMSO-d₆, 400 MHz,) δ 12.25 (s, 1H), 8.15 (d, J=11.6 Hz, 1H), 8.11 (dd, J=8.6, 5.6 Hz, 1H), 7.32 (dd, J=9.6, 2.0 Hz, 1H). 7.10 (t, J=1.2 Hz, 1H), 4.84 (split singlet, conformers, 1H), 3.63-3.45 (m, 3H), 3.36-3.27 (m, 2H), 1.92-1.73 (m, 2H), 1.34 (s, 1H, conformers), 1.24 (s, 2H, conformers).

### 1-[2-(6-fluoro-1H-indol-3-yl)ethyl]-3-methyl-pyrrolidin-3-ol

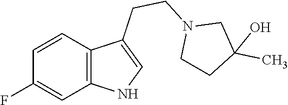 [SMILES: CC1(O)CCN(CCc2c[nH]c3cc(F)ccc23)C1]

LiAlH₄ (0.235 g, 6.20 mmol) was taken in anhydrous THF (10 mL) at 0° C. under an atmosphere of N₂ was added 1-(6-fluoro-1H-indol-3-yl)-2-(3-hydroxy-3-methyl-pyrrolidin-1-yl)ethane-1,2-dione (0.30 g, 1.03 mmol)) portion-wise under stirring. The mixture was heated to reflux (65° C.) and stirred for 8 h, then cooled to 10° C. and added dropwise to a mixture of ice-cold H₂O (10 mL) [Note: quenching was exothermic]. The mixture was filtered through celite and the filter cake was washed with EtOAc (100 mL). The filtrate was separated, the aqueous layer was extracted with EtOAc (3×30 mL). The combined organic layers were washed with brine (30 mL), dried over MgSO₄, filtered and the filtrate was removed under reduced pressure at 50° C. The crude product was purified by column chromatography on silica gel (eluent: 5% 7N NH₃ in MeOH/EtOAc) to afford the title compound (198 mg, 73%) as a viscous oil. Retention time: 1.10 min; m/z calculated for [C₁₅H₁₉FN₂O]⁺ 263.3 found 263.3 [M+H]⁺; ¹H NMR (CDCl₃, 400 MHz,) δ 7.98 (s, 1H), 7.50 (ddt, J=8.8, 5.4, 0.7 Hz, 1H), 7.08-6.81 (m, 3H), 3.15-3.00 (m, 1H), 3.00-2.72 (m, 5H), 2.45-2.24 (m, 2H), 1.98-1.81 (m, 2H), 1.38 (s, 3H).

### 1-[2-(6-fluoro-1H-indol-3-yl)ethyl]-3-methyl-pyrrolidin-3-ol fumarate salt

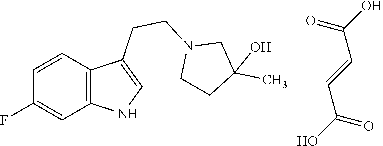 [SMILES: CC1(O)CCN(CCc2c[nH]c3cc(F)ccc23)C1 ; O=C(O)/C=C/C(=O)O]

To a mixture of 1-[2-(6-fluoro-1H-indol-3-yl)ethyl]-3-methyl-pyrrolidin-3-ol (80 mg, 0.304 mmol) in THF (2 mL) was added fumaric acid (35.4 mg, 1 eq) dissolved in THF (3 mL). At the beginning of the addition of the fumaric acid, the solution of the free base turned cloudy and then dissolved after 20 min. Some solvent was evaporated (1/3) and the precipitate formed was filtered, rinsed with Et₂O (2×2 mL) and dried under vacuum to give the fumarate salt (55 mg, 47%) as a solid. Retention time: 1.55 min; m/z calculated for [C₁₅H₁₉FN₂O]⁺ 263.3 found 263.3 [M+H]⁺; ¹H NMR (DMSO-d₆, 400 MHz) δ 10.89 (s, 1H), 7.53 (dd, J=8.8, 5.6 Hz, 1H), 7.17 (d, J=2.0 Hz, 1H), 7.10 (dd, J=10.0, 2.4 Hz), 1H), 6.87-6.81 (m, 1H), 6.54 (s, 2H, fumarate), 3.10-3.06 (m, 1H), 3.01-2.84 (m, 6H), 1.86-1.84 (m, 2H), 1.30 (s, 3H).

### 1-(5,6-difluoro-1H-indol-3-yl)-2-(3-hydroxy-3-methyl-pyrrolidin-1-yl)ethane-1,2-dione

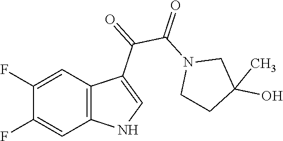 [SMILES: CC1(O)CCN(C(=O)C(=O)c2c[nH]c3cc(F)c(F)cc23)C1]

To a mixture of 3-methylpyrrolidin-3-ol (0.260 g, 2.57 mmol, 1.25 eq) in DCM (5 mL) at 0° C. was added N,N-diisopropylethylamine (0.703 mL, 4.11 mmol, 2 eq), and 2-(5-fluoro-1H-indol-3-yl)-2-oxoacetyl chloride (0.500 g, 2.05 mmol, 1 eq) in THF (10 mL) dropwise. The mixture was warmed to 15° C. and stirred for 2 h, then concentrated in vacuo and the residue triturated with H₂O. The solid was filtered, rinsed with H₂O and then with Et₂O. The solid was dried under vacuum overnight to afford the title compound (434 mg, 68%) as a solid. Retention time: 1.39 min; m/z calculated for [C₁₅H₁₄F₂N₂O₃]⁺ 309.0 found 309.0 [M+H]⁺; ¹H NMR (DMSO-d₆, 400 MHz) δ 12.35 (br. s, 1H), 8.22 (d, J=11.6 Hz, 1H), 7.97 (dd, J=11.0, 8.0 Hz, 1H), 7.6-7.55 (m, 1H), 4.84 and 4.78 (two peaks as singlet, 1H, two conformers), 3.60-3.48 (m, 2H), 3.34-3.30 (m, 2H), 1.84-1.82 (m, 2H), 1.34 and 1.24 (two peaks as singlet, 3H, two conformers).

### 1-[2-(5,6-difluoro-1H-indol-3-yl)ethyl]-3-methyl-pyrrolidin-3-ol

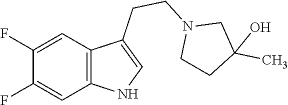 [SMILES: CC1(O)CCN(CCc2c[nH]c3cc(F)c(F)cc23)C1]

LiAlH₄ (0.222 g, 5.98 mmol) was taken in anhydrous THF (10 mL) at 0° C. under an atmosphere of N₂ was added 1-(5,6-difluoro-TH-indol-3-yl)-2-(3-hydroxy-3-methyl-pyrrolidin-1-yl)ethane-1,2-dione (0.30 g, 0.973 mmol) portion-wise with stirring. The mixture was heated to reflux (65° C.) and stirred for 8 h, then cooled to 10° C., and the mixture was added dropwise to ice-cold H₂O (10 mL) [quenching was exothermic]. To the suspension obtained was added EtOAc (50 mL) and both layers were stirred well. The suspension was filtered through a pad of celite and the solids were rinsed with EtOAc (100 mL). The filtrate was separated and the organic layer was washed with brine (2×30 mL), dried over Na₂SO₄, filtered and solvent removed under reduced pressure at 50° C. The crude product was purified by column chromatography on silica gel (eluent: 1-5% 7N NH₃ in MeOH/EtOAc) to afford the title compound (155 mg, 56%) as a solid. Retention time: 1.16 min; m/z calculated for [C₁₅H₁₈F₂N₂O]⁺ 281.3 found 281.3 [M+H]⁺; ¹H NMR (CDCl₃, 400 MHz) δ 7.98 (br. s, 1H), 7.30 (dd, J=10.8, 8.0 Hz, 1H), 7.11 (dd, J=10.6, 6.0 Hz, 1H), 7.04 (t, J=1.2 Hz, 1H), 3.10-3.04 (m, 1H), 2.89-2.74 (m, 5H), 2.40-2.36 (m, 1H), 2.34-2.28 (m, 1H), 1.92-1.89 (m, 2H), 1.37 (s, 2H).

### 1-[2-(5,6-difluoro-1H-indol-3-yl)ethyl]-3-methyl-pyrrolidin-3-ol fumarate salt

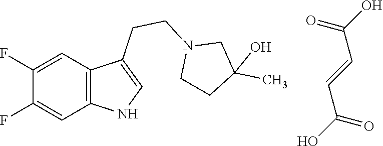 [SMILES: CC1(O)CCN(CCc2c[nH]c3cc(F)c(F)cc23)C1 ; O=C(O)/C=C/C(=O)O]

To a mixture of the free base 1-[2-(5,6-difluoro-1H-indol-3-yl)ethyl]-3-methyl-pyrrolidin-3-ol (70 mg, 0.249 mmol) in THF (1 mL) was added a solution of fumaric acid (28.9 mg, 1 eq) in THF (2 mL). The solid that precipitated was filtered and rinsed with Et₂O (2×2 mL) and dried under vacuum to give the fumarate salt (48 mg, 48%) as a solid. Retention time: 1.72 mins (10 min run); m/z calculated for [C₁₅H₁₈F₂N₂O]⁺ 281.3 found 281.3 [M+H]⁺; ¹H NMR (DMSO-d₆, 400 MHz) δ 10.95 (br. s, 1H), 7.50 (dd, J=11.4, 8.0 Hz, 1H), 7.31 (dd, J=11.4, 7.2 Hz, 1H), 7.23 (s, 1H), 6.62 (s, 1H, fumarate), 2.92-2.66 (m, 8H), 1.81-1.76 (m, 2H), 1.28 (s, 2H).

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A compound having Formula (X) 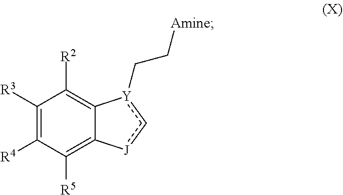
or a pharmaceutically acceptable salt or isomer thereof, the compound comprising an indole ring system, wherein
J and Y are each independently C, CH, N, or NH;
one “ [symbol] ” is a single bond and one “ [symbol] ” is a double bond;
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl;
R^{6b}, R^{6c} and R^{6d} are each independently H or C₁₋₆ alkyl; and
“Amine” represents an amine selected from: 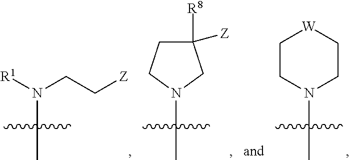
wherein
R¹ is H, C₁₋₆ alkyl, or C₄₋₁₄ alkyl-cycloalkyl;
Z is —OH, —SR^{7a}, —S(O)R^{7a}, —S(O)₂R^{7a}, —N(R^{7b}R^{7c}), or —N(R^{7b})C(O)R^{7c};
R^{7a}, R^{7b} and R^{7c} are each independently H or C₁₋₆ alkyl;
R⁸ is H or methyl;
W is —C(R^{7a})(Z); and
“ [symbol] ” indicates a point of attachment to the indole ring system,
with the proviso that the compound is not

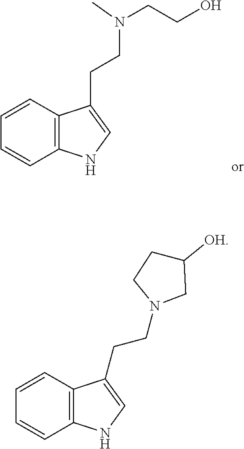 [SMILES: CN(CCO)CCc1c[nH]c2ccccc12 ; OC1CCN(CCc2c[nH]c3ccccc23)C1]

2. The compound according to claim 1 having a Formula (I) 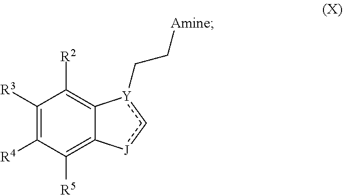
or a pharmaceutically acceptable salt or isomer thereof, the compound comprising an indole ring system, wherein
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl;
R^{6b}, R^{6c} and R^{6d} are each independently H or C₁₋₆ alkyl; and
“Amine” represents an amine selected from: 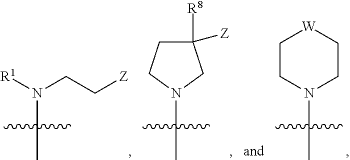
wherein
R¹ is H, C₁₋₆ alkyl, or C₄₋₁₄ alkyl-cycloalkyl;
Z is —OH, —SR^{7a}, —S(O)R^{7a}, —S(O)₂R^{7a}, —N(R^{7b}R^{7c}), or —N(R^{7b})C(O)R^{7c};
R^{7a}, R^{7b} and R^{7c} are each independently H or C₁₋₆ alkyl;
R⁸ is H or methyl;
W is —C(R^{7a})(Z); and
“ [symbol] ” indicates a point of attachment to the indole ring system.

3. The compound according to claim 1 or 2, wherein the Amine is 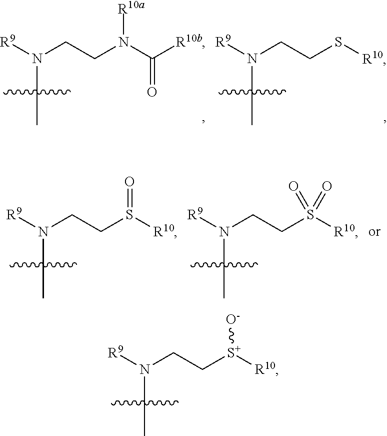
and R⁹, R¹⁰, R^{10a}, and R^{10b} are each independently selected from H, C₁₋₆ alkyl, and C₄₋₁₄ alkyl-cycloalkyl.

4. The compound according to any one of claims 1-3, wherein the Amine is 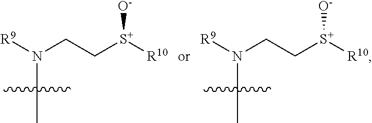
and R⁹ and R¹⁰ are each independently selected from H, C₁₋₆ alkyl, and C₄₋₁₄ alkyl-cycloalkyl.

5. The compound according to claim 3 or 4, wherein R¹⁰, R^{10a}, and R^{10b} are methyl.

6. The compound according to claim 1 or 2, wherein the Amine has a formula 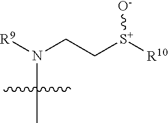
and R⁹ and R¹⁰ are each independently selected from C₁₋₆ alkyl.

7. The compound according to claim 6, wherein the Amine has a formula

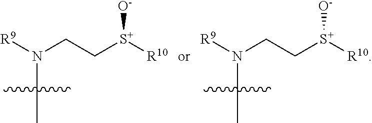

8. The compound according to claim 1 or 2, wherein the Amine is 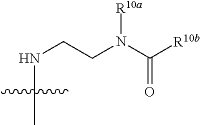
and R^{10a} and R^{10b} are each independently selected from H, C₁₋₆ alkyl, and C₄₋₁₄ alkyl-cycloalkyl.

9. The compound according to claim 8, wherein R^{10a} and R^{10b} are methyl.

10. The compound according to any one of claims 1-9, wherein the compound is

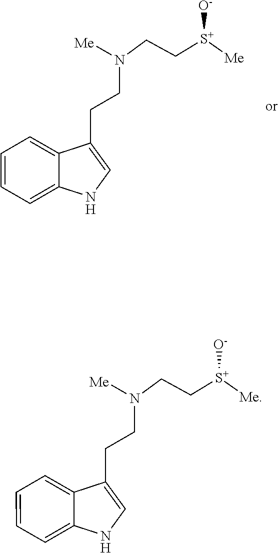 [SMILES: CN(CCc1c[nH]c2ccccc12)CC[S@+](C)[O-] ; CN(CCc1c[nH]c2ccccc12)CC[S@@+](C)[O-]]

11. The compound according to claim 1 or 2, wherein the Amine is 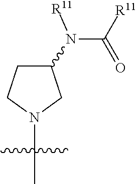
and each R¹¹ independently is H or C₁₋₆ alkyl.

12. The compound according to claim 11, wherein the Amine is

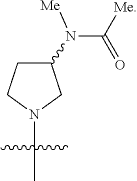 [SMILES: *N1CCC(N(C)C(C)=O)C1]

13. The compound according to claim 12, wherein the Amine is

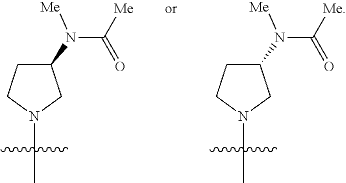 [SMILES: *N1CC[C@@H](N(C)C(C)=O)C1 ; *N1CC[C@H](N(C)C(C)=O)C1]

14. The compound according to any one of claims 11-13, wherein the compound

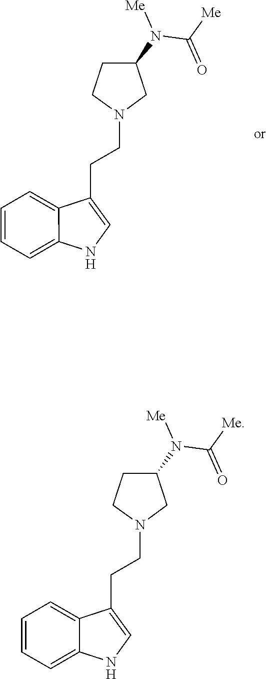 [SMILES: CC(=O)N(C)[C@@H]1CCN(CCc2c[nH]c3ccccc23)C1 ; CC(=O)N(C)[C@H]1CCN(CCc2c[nH]c3ccccc23)C1]

15. The compound according to claim 1 or 2, wherein the Amine is 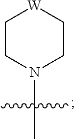
W is —C(R^{7a})(Z); R^{7a} is H or C₁₋₆ alkyl; and Z is —OH.

16. The compound according to claim 15, wherein the Amine is

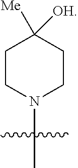 [SMILES: *N1CCC(C)(O)CC1]

17. The compound according to claim 15 or 16, wherein the compound is

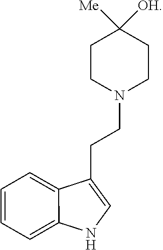 [SMILES: CC1(O)CCN(CCc2c[nH]c3ccccc23)CC1]

18. The compound according to claim 1 having a Formula (II) 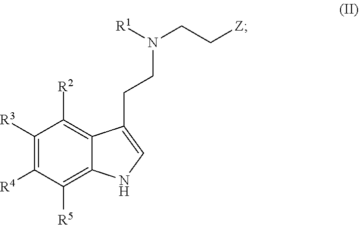
or a pharmaceutically acceptable salt or isomer thereof, wherein
R¹ is H, C₁₋₆ alkyl, or C₄₋₁₄ alkyl-cycloalkyl;
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl;
R^{6b}, R^{6c} and R^{6d} are each independently H or C₁₋₆ alkyl; and
Z is —OH, —SR^{7a}, —S(O)R^{7a}, —S(O)₂R^{7a}, —N(R^{7b}R^{7c}), or —N(R^{7b})C(O)R^{7c}, where R^{7a}, R^{7b} and R^{7c} are each independently H or C₁₋₆ alkyl.

19. The compound according to claim 18, wherein R¹ is methyl, Z is —S(O)R^{7a}, R^{7a} is methyl, and R², R³, R⁴ and R⁵ are H.

20. The compound according to claim 18 or 19, wherein the compound is

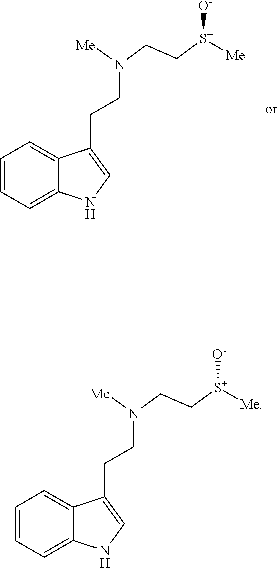 [SMILES: CN(CCc1c[nH]c2ccccc12)CC[S@+](C)[O-] ; CN(CCc1c[nH]c2ccccc12)CC[S@@+](C)[O-]]

21. The compound according to claim 1 having a Formula (III) 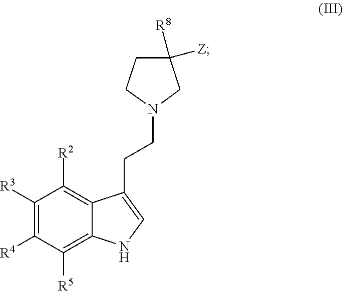
or a pharmaceutically acceptable salt or isomer thereof, wherein
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl;
R^{6b}, R^{6c} and R^{6d} are each independently H or C₁₋₆ alkyl;
R⁸ is H or methyl; and
Z is —OH, —SR^{7a}, —S(O)R^{7a}, —S(O)₂R^{7a}, —N(R^{7b}R^{7c}), or —N(R^{7b})C(O)R^{7c}, where R^{7a}, R^{7b} and R^{7c} are each independently H or C₁₋₆ alkyl.

22. The compound according to claim 21, wherein Z is —N(R^{7b})C(O)R^{7c}, R^{7b} and R^{7c} are Me, and R², R³, R⁴ and R⁵ are H.

23. The compound according to claim 21 or 22, wherein the compound is

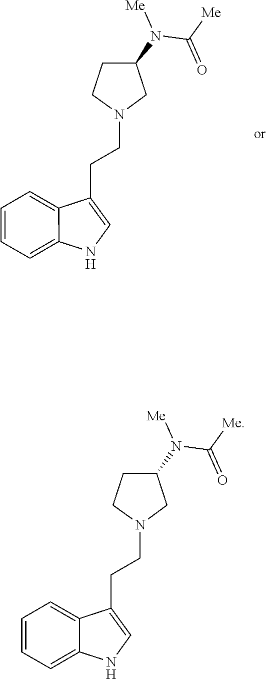 [SMILES: CC(=O)N(C)[C@@H]1CCN(CCc2c[nH]c3ccccc23)C1 ; CC(=O)N(C)[C@H]1CCN(CCc2c[nH]c3ccccc23)C1]

24. The compound according to claim 1 having a Formula (IV) 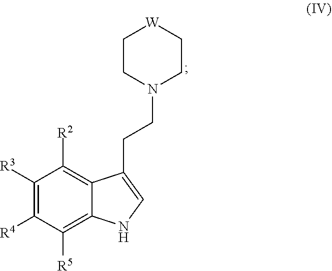
or a pharmaceutically acceptable salt or isomer thereof, wherein
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₈ alkyl-aryl, C₅₋₁₀ heteroaryl, or C₆₋₁₆ alkyl-heteroaryl;
R^{6b}, R^{6c} and R^{6d} are each independently H or C₁₋₆ alkyl;
W is —C(R^{7a})(Z);
Z is OH, S, —S(O), —S(O)₂, —N(R^{7a}), or —NC(O)R^{7a}; and
R^{7a} is H or C₁₋₆ alkyl.

25. The compound according to claim 24, wherein W is —C(R^{7a})(OH), R^{7a} is methyl and R², R³, R⁴ and R⁵ are H.

26. The compound according to claim 24 or 25, wherein the compound is

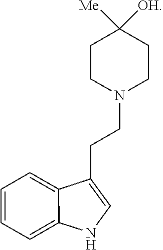 [SMILES: CC1(O)CCN(CCc2c[nH]c3ccccc23)CC1]

27. The compound according to claim 1 having a Formula (V) 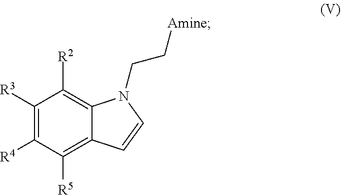
or a pharmaceutically acceptable salt or isomer thereof, the compound comprising an indole ring system, wherein
R², R³, R⁴ and R⁵ are each independently H, C₁₋₆ alkyl, C₂₋₆ alkenyl, C₂₋₆ alkynyl, halogen, C₁₋₆ haloalkyl, C₁₋₆ alkylamine, C₁₋₆ alkoxy, C₁₋₆ haloalkoxy, —OR^{6a}, —NO₂, —CN, —C(O)R^{6b}, —C(O)OR^{6b}, —OC(O)R^{6b}, —OC(O)OR^{6b}, —N(R^{6b}R^{6c}), —N(R^{6b})C(O)R^{6c}, —C(O)N(R^{6b}R^{6c}), —N(R^{6b})C(O)OR^{6c}, —OC(O)N(R^{6b}R^{6c}), —N(R^{6b})C(O)N(R^{6c}R^{6d}), —C(O)C(O)N(R^{6b}R^{6c}), —S(O₂)R^{6b}, —S(O)₂N(R^{6b}R^{6c}), C₃₋₈ cycloalkyl, C₄₋₁₄ alkyl-cycloalkyl, C₄₋heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl, or alternatively, R² and R³, R³ and R⁴, or R⁴ and R⁵ are combined with the atoms to which they are each attached to form a C₄₋₆ cycloalkyl, C₄₋₆ heterocycloalkyl, C₆₋₁₂ aryl, or C₅₋₁₀ heteroaryl;
R^{6a} is C₃₋₈ cycloalkyl, C₄₋₁₄ alkyl-cycloalkyl, C₄₋₁₀ heterocycloalkyl, C₄₋₁₆ alkyl-heterocycloalkyl, C₆₋₁₂ aryl, C₇₋₁₅ alkyl-aryl, C₅₋₁₀ heteroaryl, or C₆₋₁₆ alkyl-heteroaryl;
R^{6b}, R^{6c} and R^{6d} are each independently H or C₁₋₆ alkyl; and
“Amine” represents an amine selected from: 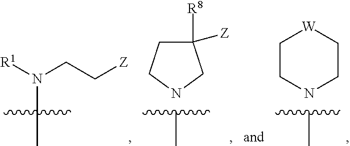
wherein
R¹ is H, C₁₋₆ alkyl, or C₄₋₁₄ alkyl-cycloalkyl;
Z is —OH, —SR^{7a}, —S(O)R^{7a}, —S(O)₂R^{7a}, —N(R^{7b}R^{7c}), or —N(R^{7b})C(O)R^{7c};
R^{7a}, R^{7b} and R^{7c} are each independently H or C₁₋₆ alkyl;
R⁸ is H or methyl;
W is —C(R^{7a})(Z); and
“ [symbol] ” indicates a point of attachment to the indole ring system.

28. The compound according to claim 1 having a Formula (I.a) 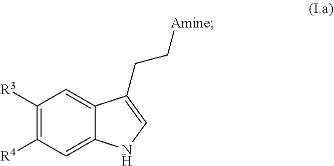
or a pharmaceutically acceptable salt or isomer thereof, wherein
R³ and R⁴ are each independently H, C₁₋₆ alkyl, or halogen;
“Amine” represents an amine that is 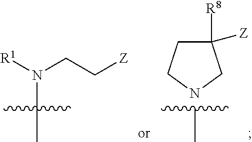
R¹ is H, C₁₋₆ alkyl, or C₄₋₁₄ alkyl-cycloalkyl;
R⁸ is H or methyl;
Z is —OH, —SR^{7a}, —S(O)R^{7a}, —S(O)₂R^{7a}, —N(R^{7b}R^{7c}), or —N(R^{7b})C(O)R^{7c}; and
R^{7a}, R^{7b} and R^{7c} are each independently H or C₁₋₆ alkyl.

29. The compound according to claim 28, wherein R³ and R⁴ are each independently H or halogen; the amine is 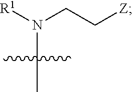
R¹ is H or methyl; Z is —OH or S(O)₂R^{7a}; and R^{7a} is H or C₁₋₆ alkyl.

30. The compound according to claim 28, wherein R³ and R⁴ are each independently H or halogen; the amine is 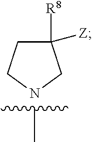
R⁸ is H or methyl; and Z is —OH.

31. The compound according to any one of claims 28-30, wherein R³ and R⁴ are each independently H or F; R¹ is methyl; and R⁸ is H or methyl.

32. The compound according to any one of claims 28-31, wherein R³ is H and R⁴ is F, R³ is F and R⁴ is H, or R³ and R⁴ are F.

33. The compound according to claim 1 having a Formula (V.a) 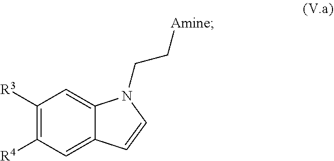
or a pharmaceutically acceptable salt or isomer thereof, wherein
R³ and R⁴ are each independently H, C₁₋₆ alkyl, or halogen;
“Amine” represents an amine that is 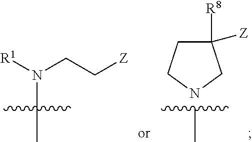
R¹ is H, C₁₋₆ alkyl, or C₄₋₁₄ alkyl-cycloalkyl;
R⁸ is H or methyl;
Z is —OH, —SR^{7a}, —S(O)R^{7a}, —S(O)₂R^{7a}, —N(R^{7b}R^{7c}), or —N(R^{7b})C(O)R^{7c}; and
R^{7a}, R^{7b} and R^{7c} are each independently H or C₁₋₆ alkyl.

34. The compound according to claim 33, wherein R³ and R⁴ are each independently H or halogen; the amine is 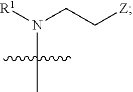
R¹ is H or methyl; Z is —OH or S(O)₂R^{7a}; and R^{7a} is H or C₁₋₆ alkyl.

35. The compound according to claim 33, wherein R³ and R⁴ are each independently H or halogen; the amine is 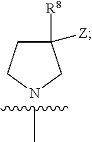
R⁸ is H or methyl; and Z is —OH.

36. The compound according to any one of claims 33-35, wherein R³ and R⁴ are each independently H or F; R¹ is methyl; and R⁸ is H or methyl.

37. The compound according to any one of claims 33-36, wherein R³ is H and R⁴ is F, R³ is F and R⁴ is H, or R³ and R⁴ are F.

38. The compound according to claim 1, or a pharmaceutically acceptable salt thereof, that is:
(S)—N-(2-(1H-indol-3-yl)ethyl)-N-methyl-2-(methylsulfinyl)ethan-1-amine;
(R)—N-(2-(1H-indol-3-yl)ethyl)-N-methyl-2-(methylsulfinyl)ethan-1-amine;
(R)—N-(1-(2-(1H-indol-3-yl)ethyl)pyrrolidin-3-yl)-N-methylacetamide;
(S)—N-(1-(2-(1H-indol-3-yl)ethyl)pyrrolidin-3-yl)-N-methylacetamide; or
1-(2-(1H-indol-3-yl)ethyl)-4-methylpiperidin-4-ol.

39. The compound according to claim 1, or a pharmaceutically acceptable salt thereof, that is:

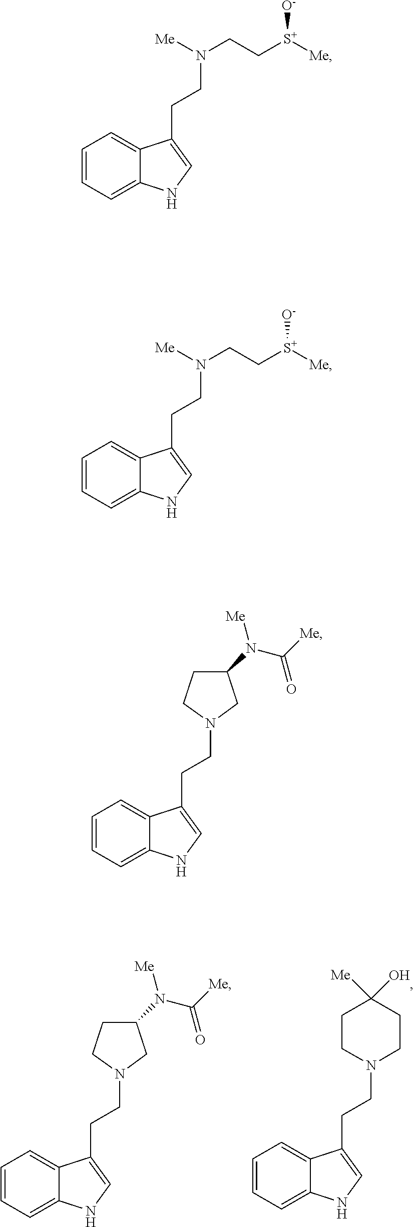 [SMILES: CN(CCc1c[nH]c2ccccc12)CC[S@+](C)[O-] ; CN(CCc1c[nH]c2ccccc12)CC[S@@+](C)[O-] ; CC(=O)N(C)[C@@H]1CCN(CCc2c[nH]c3ccccc23)C1 ; CC(=O)N(C)[C@H]1CCN(CCc2c[nH]c3ccccc23)C1 ; CC1(O)CCN(CCc2c[nH]c3ccccc23)CC1]

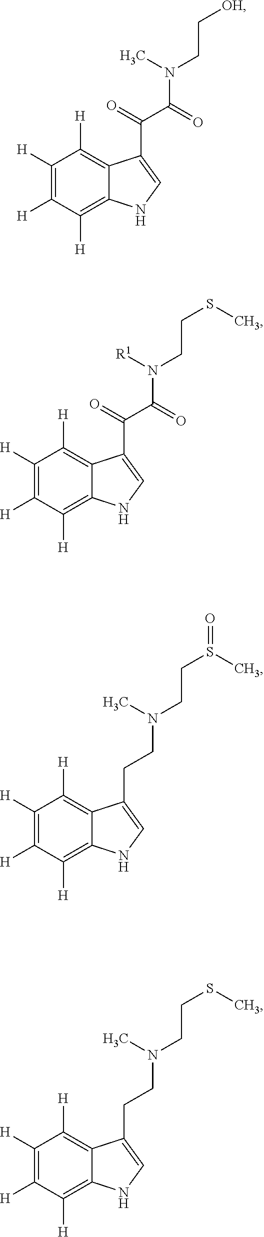

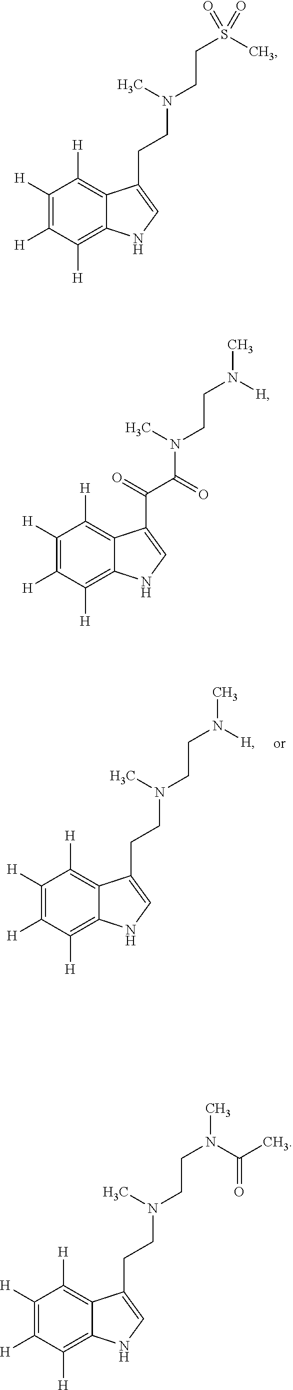

40. The compound according to claim 1, or a pharmaceutically acceptable salt thereof, that is:

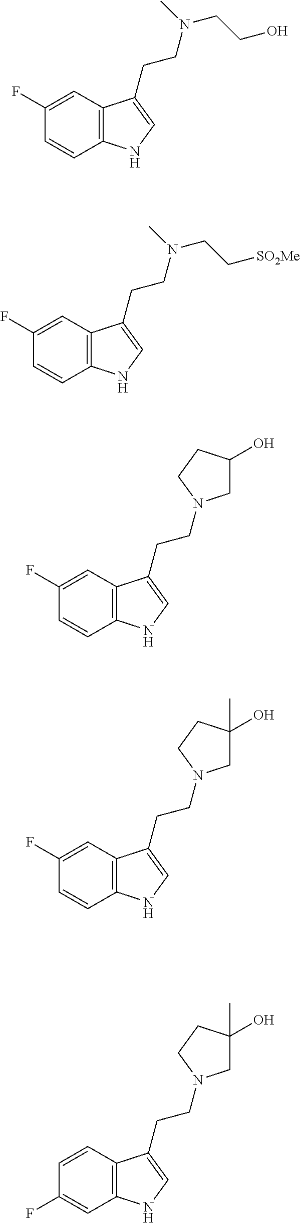 [SMILES: CN(CCO)CCc1c[nH]c2ccc(F)cc12 ; CN(CCc1c[nH]c2ccc(F)cc12)CCS(C)(=O)=O ; OC1CCN(CCc2c[nH]c3ccc(F)cc23)C1 ; CC1(O)CCN(CCc2c[nH]c3ccc(F)cc23)C1 ; CC1(O)CCN(CCc2c[nH]c3cc(F)ccc23)C1]

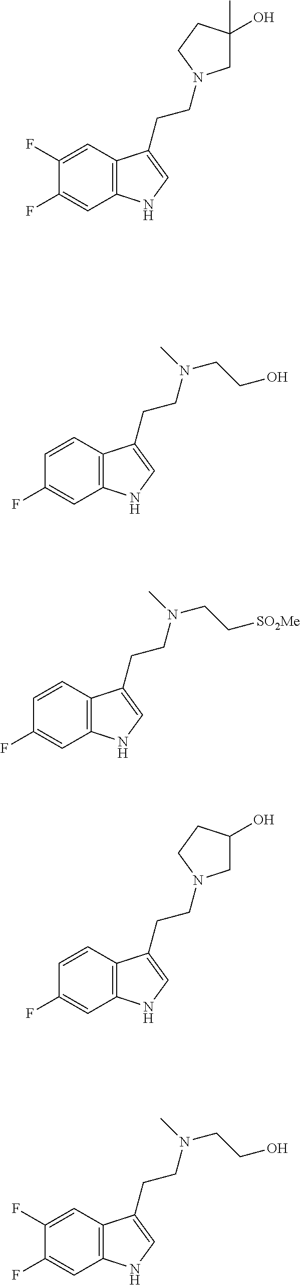 [SMILES: CC1(O)CCN(CCc2c[nH]c3cc(F)c(F)cc23)C1 ; CN(CCO)CCc1c[nH]c2cc(F)ccc12 ; CN(CCc1c[nH]c2cc(F)ccc12)CCS(C)(=O)=O ; OC1CCN(CCc2c[nH]c3cc(F)ccc23)C1 ; CN(CCO)CCc1c[nH]c2cc(F)c(F)cc12]

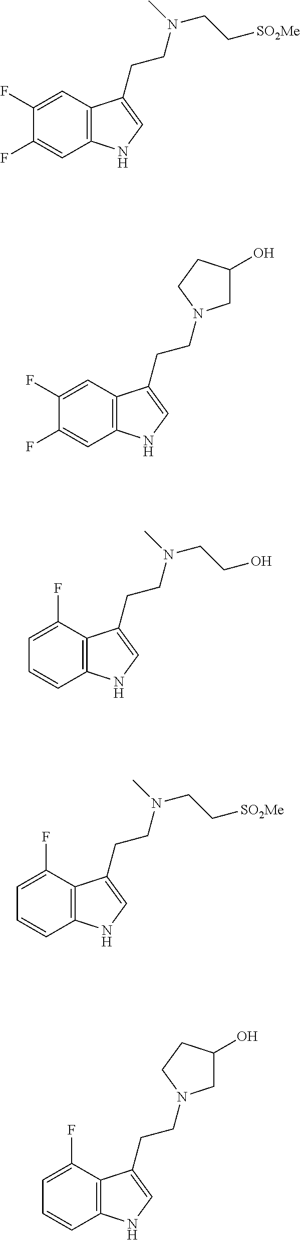 [SMILES: CN(CCc1c[nH]c2cc(F)c(F)cc12)CCS(C)(=O)=O ; OC1CCN(CCc2c[nH]c3cc(F)c(F)cc23)C1 ; CN(CCO)CCc1c[nH]c2cccc(F)c12 ; CN(CCc1c[nH]c2cccc(F)c12)CCS(C)(=O)=O ; OC1CCN(CCc2c[nH]c3cccc(F)c23)C1]

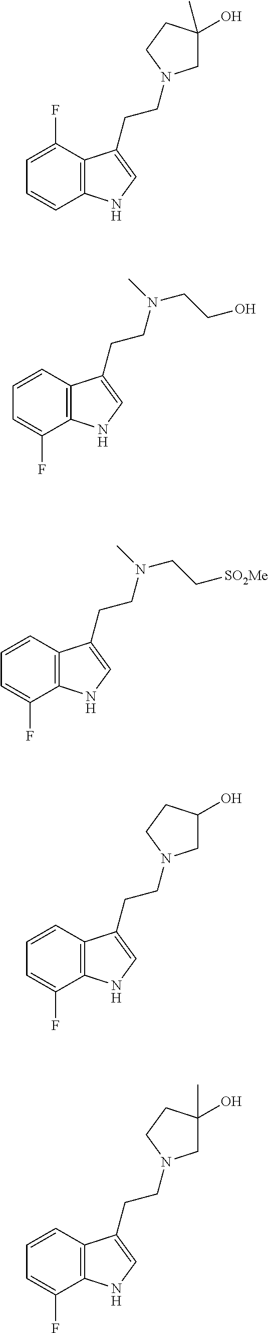 [SMILES: CC1(O)CCN(CCc2c[nH]c3cccc(F)c23)C1 ; CN(CCO)CCc1c[nH]c2c(F)cccc12 ; CN(CCc1c[nH]c2c(F)cccc12)CCS(C)(=O)=O ; OC1CCN(CCc2c[nH]c3c(F)cccc23)C1 ; CC1(O)CCN(CCc2c[nH]c3c(F)cccc23)C1]

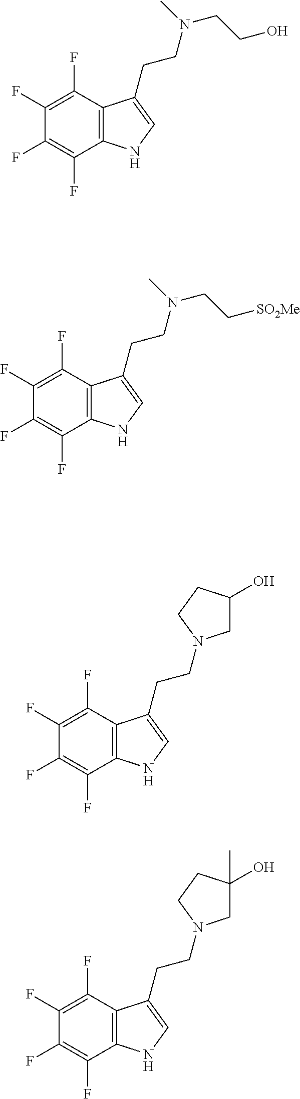 [SMILES: CN(CCO)CCc1c[nH]c2c(F)c(F)c(F)c(F)c12 ; CN(CCc1c[nH]c2c(F)c(F)c(F)c(F)c12)CCS(C)(=O)=O ; OC1CCN(CCc2c[nH]c3c(F)c(F)c(F)c(F)c23)C1 ; CC1(O)CCN(CCc2c[nH]c3c(F)c(F)c(F)c(F)c23)C1]

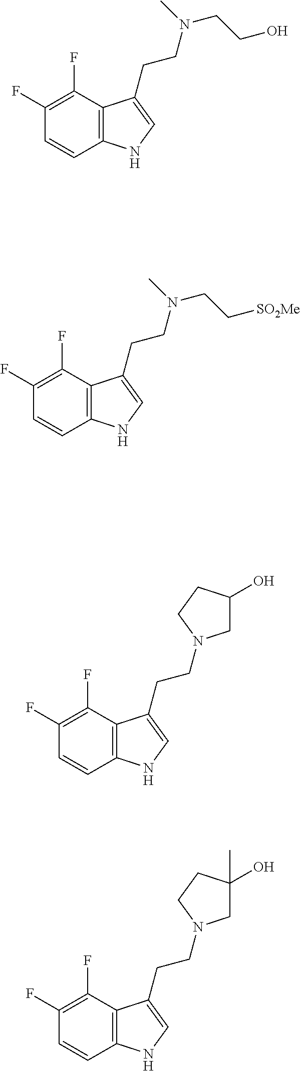 [SMILES: CN(CCO)CCc1c[nH]c2ccc(F)c(F)c12 ; CN(CCc1c[nH]c2ccc(F)c(F)c12)CCS(C)(=O)=O ; OC1CCN(CCc2c[nH]c3ccc(F)c(F)c23)C1 ; CC1(O)CCN(CCc2c[nH]c3ccc(F)c(F)c23)C1]

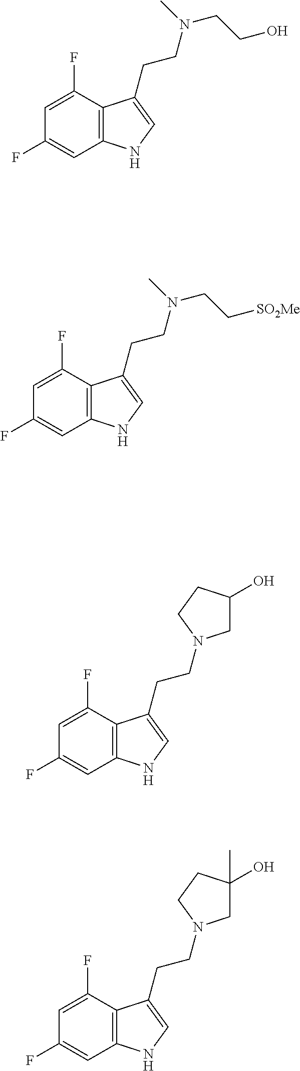 [SMILES: CN(CCO)CCc1c[nH]c2cc(F)cc(F)c12 ; CN(CCc1c[nH]c2cc(F)cc(F)c12)CCS(C)(=O)=O ; OC1CCN(CCc2c[nH]c3cc(F)cc(F)c23)C1 ; CC1(O)CCN(CCc2c[nH]c3cc(F)cc(F)c23)C1]

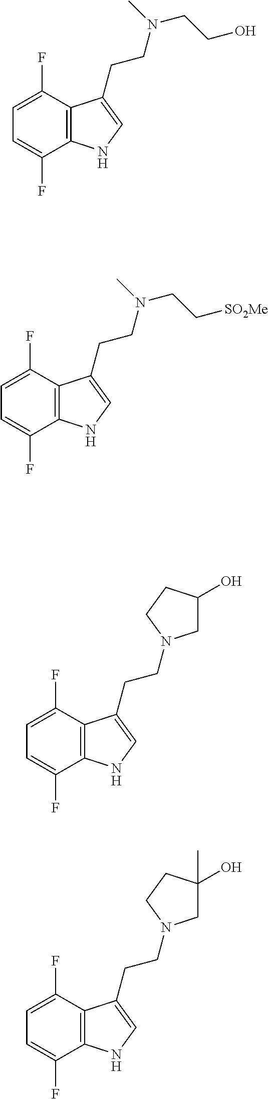 [SMILES: CN(CCO)CCc1c[nH]c2c(F)ccc(F)c12 ; CN(CCc1c[nH]c2c(F)ccc(F)c12)CCS(C)(=O)=O ; OC1CCN(CCc2c[nH]c3c(F)ccc(F)c23)C1 ; CC1(O)CCN(CCc2c[nH]c3c(F)ccc(F)c23)C1]

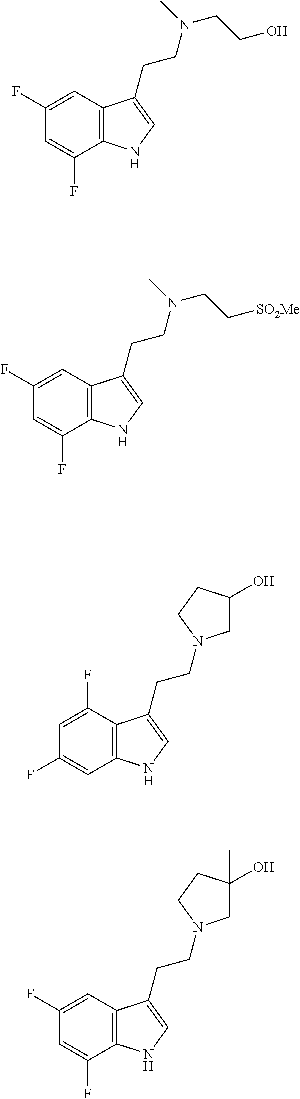 [SMILES: CN(CCO)CCc1c[nH]c2c(F)cc(F)cc12 ; CN(CCc1c[nH]c2c(F)cc(F)cc12)CCS(C)(=O)=O ; OC1CCN(CCc2c[nH]c3cc(F)cc(F)c23)C1 ; CC1(O)CCN(CCc2c[nH]c3c(F)cc(F)cc23)C1]

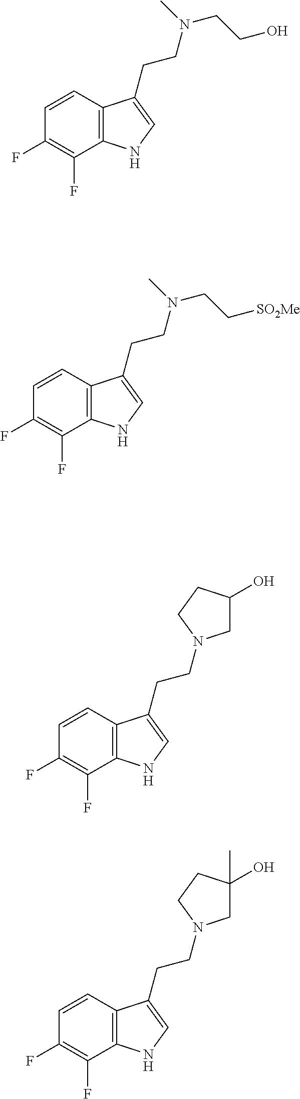 [SMILES: CN(CCO)CCc1c[nH]c2c(F)c(F)ccc12 ; CN(CCc1c[nH]c2c(F)c(F)ccc12)CCS(C)(=O)=O ; OC1CCN(CCc2c[nH]c3c(F)c(F)ccc23)C1 ; CC1(O)CCN(CCc2c[nH]c3c(F)c(F)ccc23)C1]

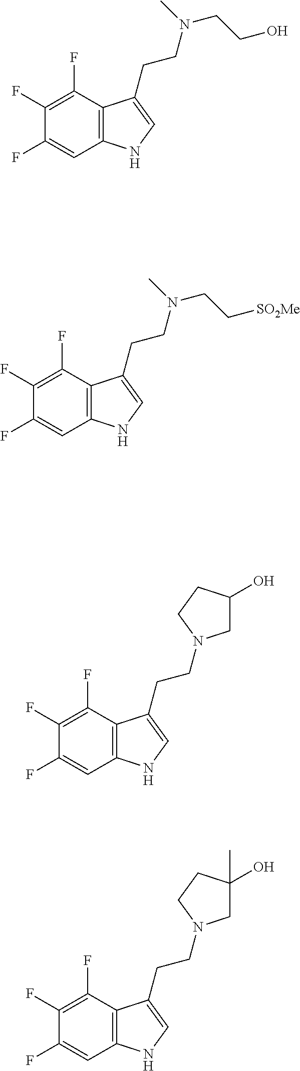 [SMILES: CN(CCO)CCc1c[nH]c2cc(F)c(F)c(F)c12 ; CN(CCc1c[nH]c2cc(F)c(F)c(F)c12)CCS(C)(=O)=O ; OC1CCN(CCc2c[nH]c3cc(F)c(F)c(F)c23)C1 ; CC1(O)CCN(CCc2c[nH]c3cc(F)c(F)c(F)c23)C1]

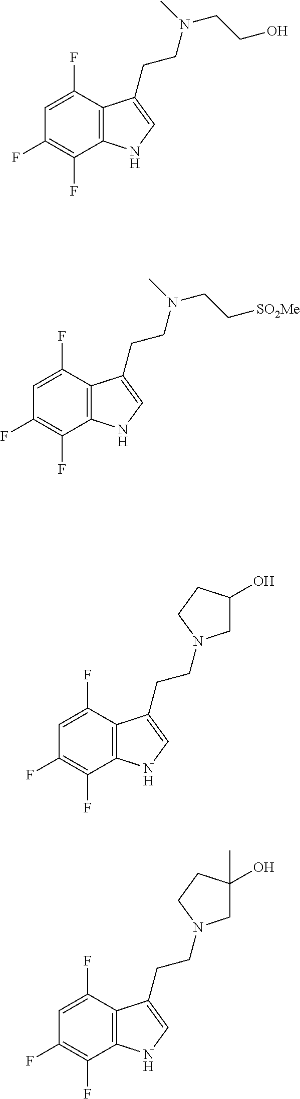 [SMILES: CN(CCO)CCc1c[nH]c2c(F)c(F)cc(F)c12 ; CN(CCc1c[nH]c2c(F)c(F)cc(F)c12)CCS(C)(=O)=O ; OC1CCN(CCc2c[nH]c3c(F)c(F)cc(F)c23)C1 ; CC1(O)CCN(CCc2c[nH]c3c(F)c(F)cc(F)c23)C1]

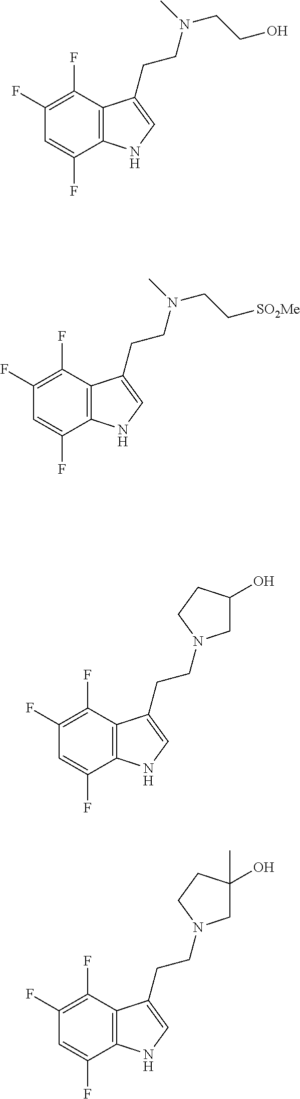 [SMILES: CN(CCO)CCc1c[nH]c2c(F)cc(F)c(F)c12 ; CN(CCc1c[nH]c2c(F)cc(F)c(F)c12)CCS(C)(=O)=O ; OC1CCN(CCc2c[nH]c3c(F)cc(F)c(F)c23)C1 ; CC1(O)CCN(CCc2c[nH]c3c(F)cc(F)c(F)c23)C1]

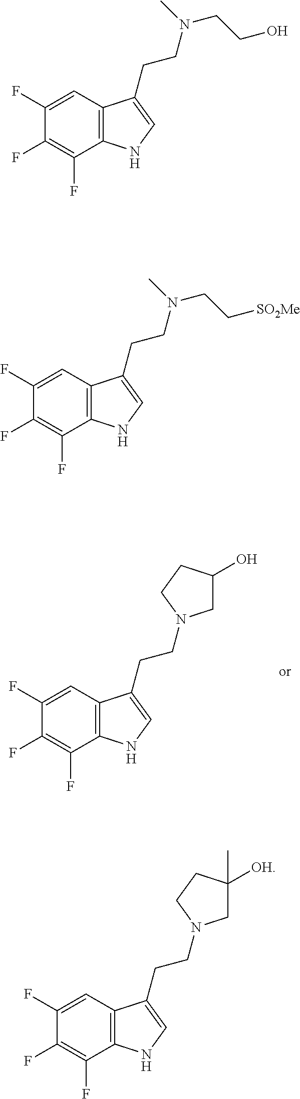 [SMILES: CN(CCO)CCc1c[nH]c2c(F)c(F)c(F)cc12 ; CN(CCc1c[nH]c2c(F)c(F)c(F)cc12)CCS(C)(=O)=O ; OC1CCN(CCc2c[nH]c3c(F)c(F)c(F)cc23)C1 ; CC1(O)CCN(CCc2c[nH]c3c(F)c(F)c(F)cc23)C1]

41. The compound according to claim 1, or a pharmaceutically acceptable salt thereof, that is:

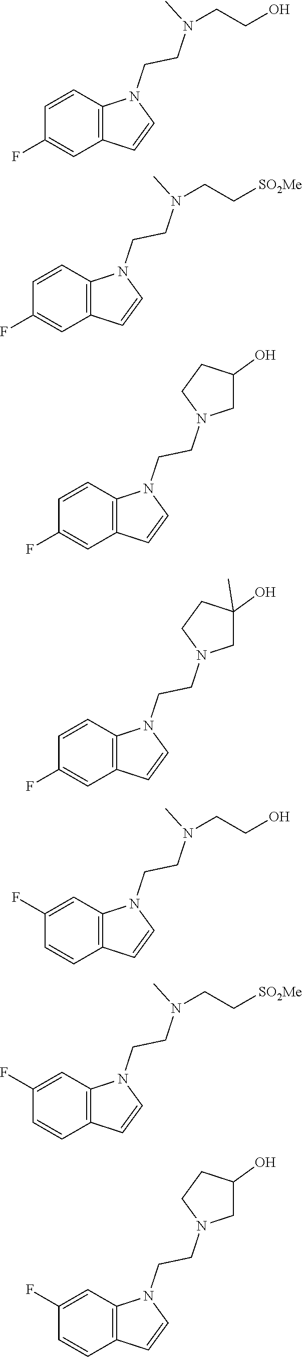

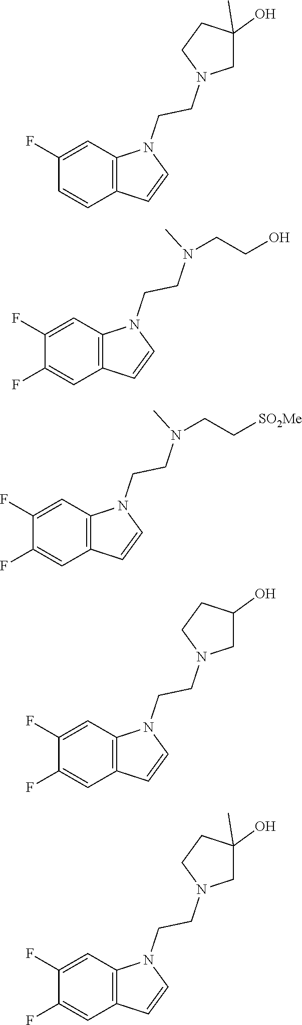

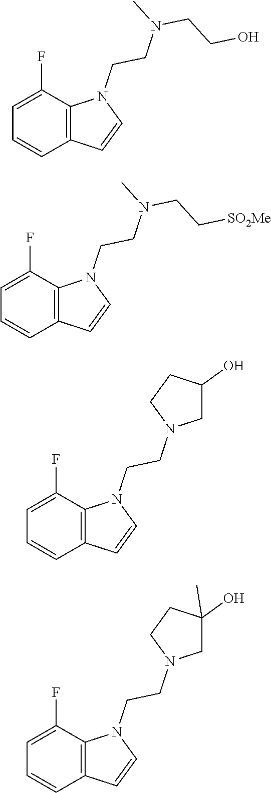

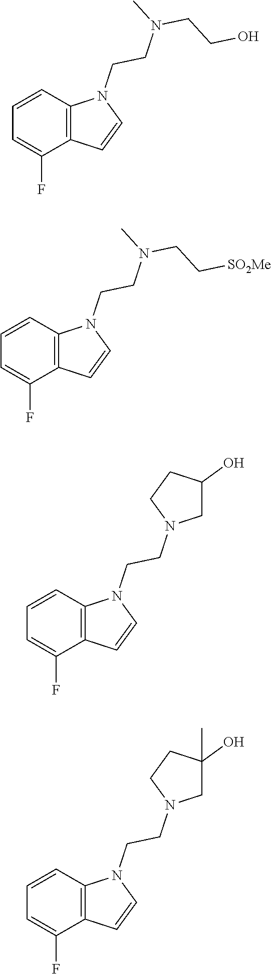 [SMILES: CN(CCO)CCn1ccc2c(F)cccc21 ; CN(CCn1ccc2c(F)cccc21)CCS(C)(=O)=O ; OC1CCN(CCn2ccc3c(F)cccc32)C1 ; CC1(O)CCN(CCn2ccc3c(F)cccc32)C1]

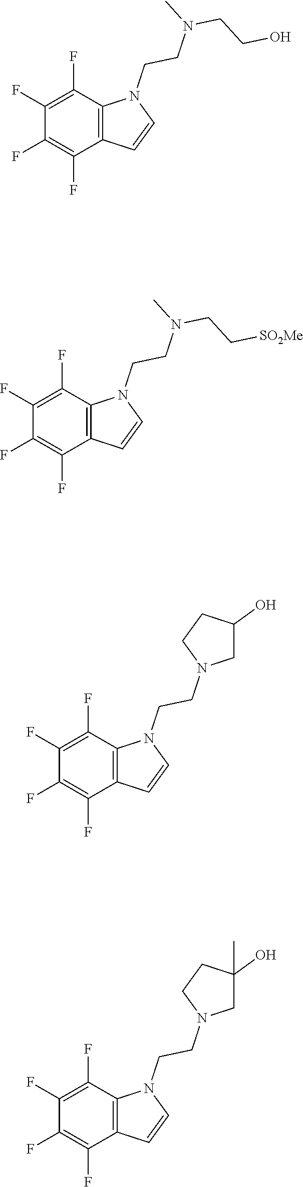 [SMILES: CN(CCO)CCn1ccc2c(F)c(F)c(F)c(F)c21 ; CN(CCn1ccc2c(F)c(F)c(F)c(F)c21)CCS(C)(=O)=O ; OC1CCN(CCn2ccc3c(F)c(F)c(F)c(F)c32)C1 ; CC1(O)CCN(CCn2ccc3c(F)c(F)c(F)c(F)c32)C1]

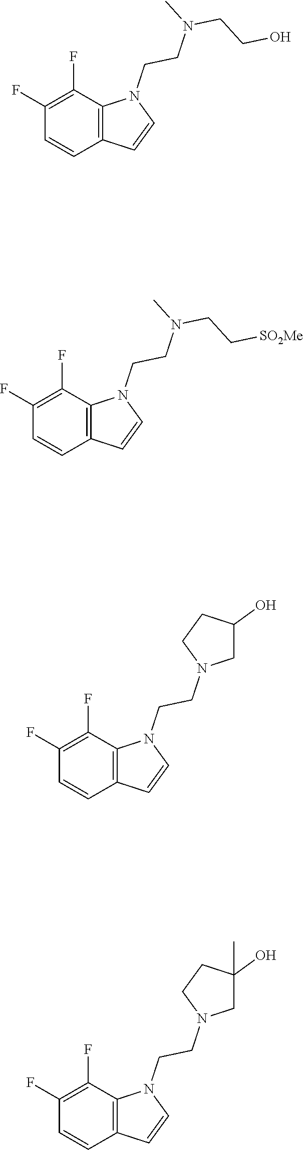 [SMILES: CN(CCO)CCn1ccc2ccc(F)c(F)c21 ; CN(CCn1ccc2ccc(F)c(F)c21)CCS(C)(=O)=O ; OC1CCN(CCn2ccc3ccc(F)c(F)c32)C1 ; CC1(O)CCN(CCn2ccc3ccc(F)c(F)c32)C1]

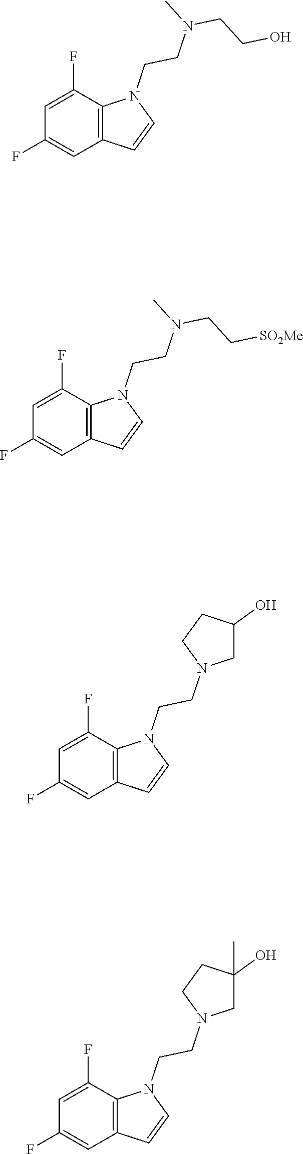 [SMILES: CN(CCO)CCn1ccc2cc(F)cc(F)c21 ; CN(CCn1ccc2cc(F)cc(F)c21)CCS(C)(=O)=O ; OC1CCN(CCn2ccc3cc(F)cc(F)c32)C1 ; CC1(O)CCN(CCn2ccc3cc(F)cc(F)c32)C1]

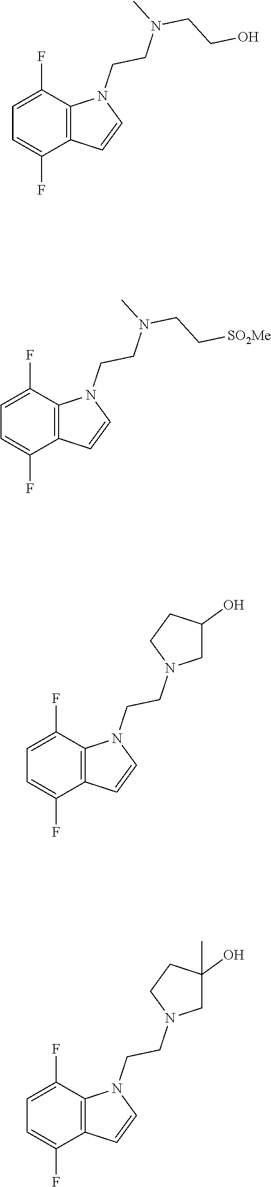 [SMILES: CN(CCO)CCn1ccc2c(F)ccc(F)c21 ; CN(CCn1ccc2c(F)ccc(F)c21)CCS(C)(=O)=O ; OC1CCN(CCn2ccc3c(F)ccc(F)c32)C1 ; CC1(O)CCN(CCn2ccc3c(F)ccc(F)c32)C1]

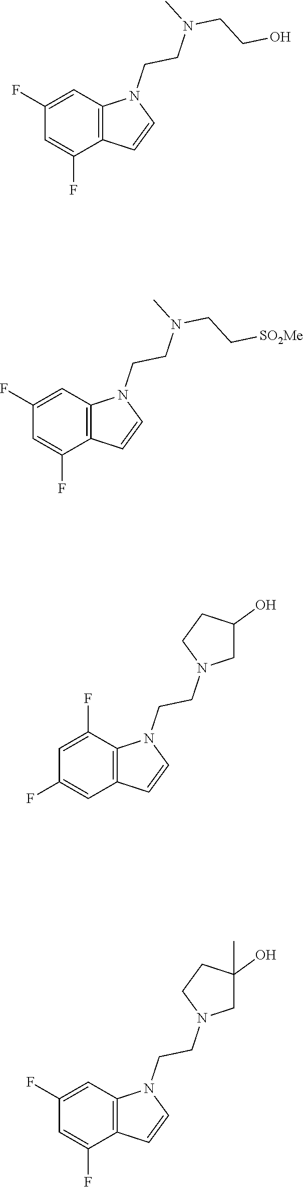 [SMILES: CN(CCO)CCn1ccc2c(F)cc(F)cc21 ; CN(CCn1ccc2c(F)cc(F)cc21)CCS(C)(=O)=O ; OC1CCN(CCn2ccc3cc(F)cc(F)c32)C1 ; CC1(O)CCN(CCn2ccc3c(F)cc(F)cc32)C1]

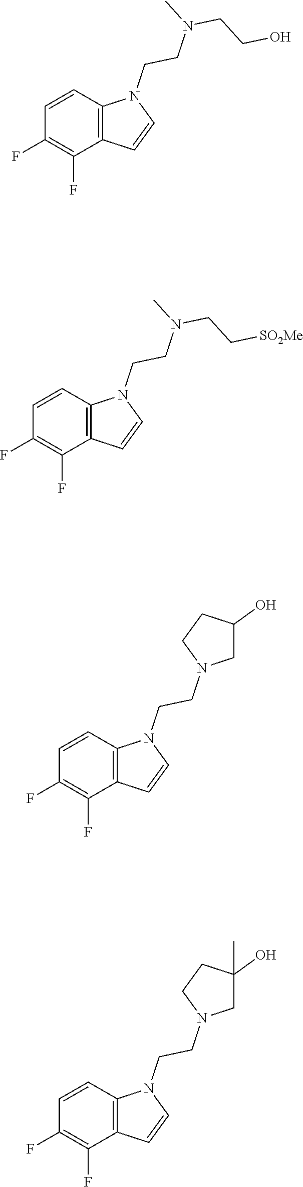 [SMILES: CN(CCO)CCn1ccc2c(F)c(F)ccc21 ; CN(CCn1ccc2c(F)c(F)ccc21)CCS(C)(=O)=O ; OC1CCN(CCn2ccc3c(F)c(F)ccc32)C1 ; CC1(O)CCN(CCn2ccc3c(F)c(F)ccc32)C1]

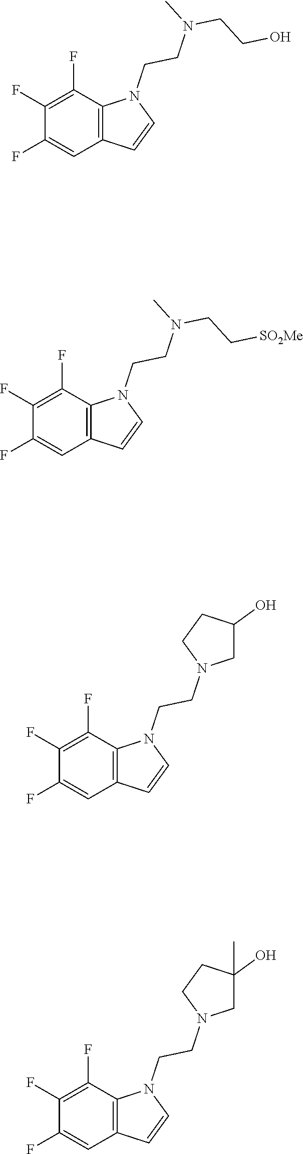 [SMILES: CN(CCO)CCn1ccc2cc(F)c(F)c(F)c21 ; CN(CCn1ccc2cc(F)c(F)c(F)c21)CCS(C)(=O)=O ; OC1CCN(CCn2ccc3cc(F)c(F)c(F)c32)C1 ; CC1(O)CCN(CCn2ccc3cc(F)c(F)c(F)c32)C1]

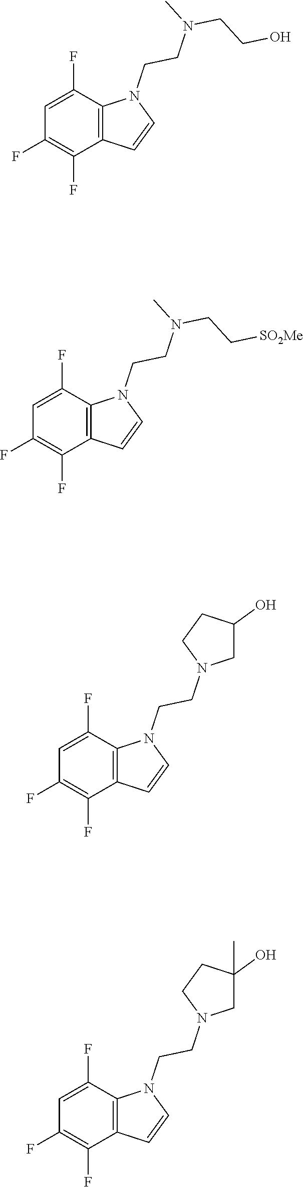 [SMILES: CN(CCO)CCn1ccc2c(F)c(F)cc(F)c21 ; CN(CCn1ccc2c(F)c(F)cc(F)c21)CCS(C)(=O)=O ; OC1CCN(CCn2ccc3c(F)c(F)cc(F)c32)C1 ; CC1(O)CCN(CCn2ccc3c(F)c(F)cc(F)c32)C1]

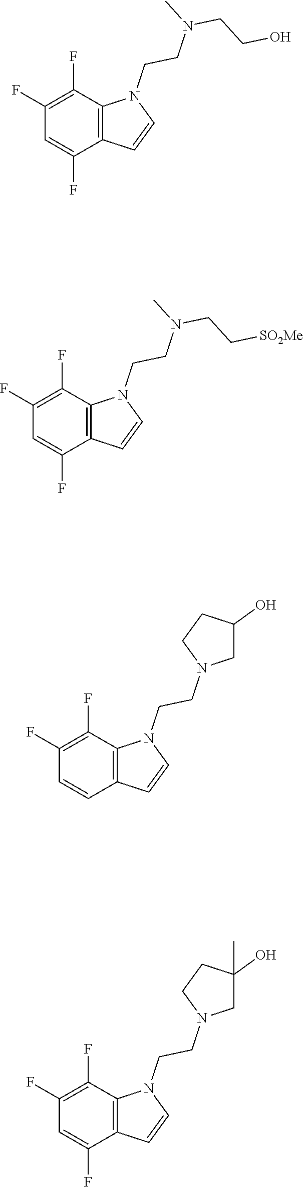 [SMILES: CN(CCO)CCn1ccc2c(F)cc(F)c(F)c21 ; CN(CCn1ccc2c(F)cc(F)c(F)c21)CCS(C)(=O)=O ; OC1CCN(CCn2ccc3ccc(F)c(F)c32)C1 ; CC1(O)CCN(CCn2ccc3c(F)cc(F)c(F)c32)C1]

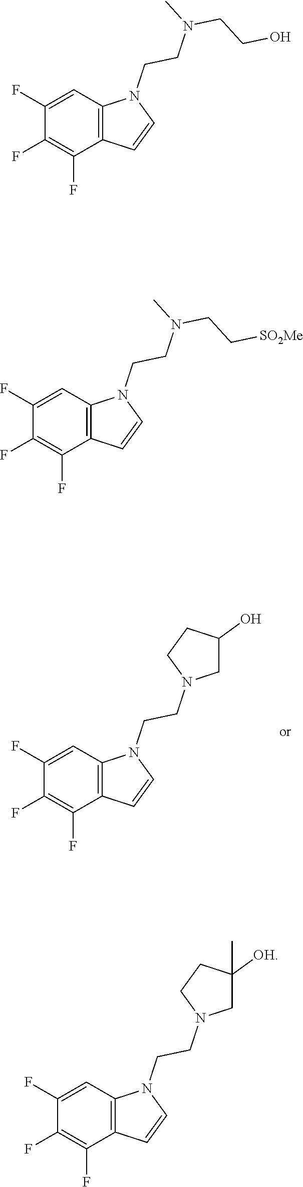 [SMILES: CN(CCO)CCn1ccc2c(F)c(F)c(F)cc21 ; CN(CCn1ccc2c(F)c(F)c(F)cc21)CCS(C)(=O)=O ; OC1CCN(CCn2ccc3c(F)c(F)c(F)cc32)C1 ; CC1(O)CCN(CCn2ccc3c(F)c(F)c(F)cc32)C1]

42. A composition comprising a compound according to any one of claims 1-41 and further comprising an excipient, an adjuvant, a carrier, or combinations thereof.

43. The composition according to claim 42, comprising two or more compounds according to any one of claims 1-41.

44. The composition according to claim 42 or 43, further comprising at least one additional biologically active compound.

45. The composition according to any one of claims 42-44, wherein the composition is a solid form selected from a powder, tablet, pill, capsule, cachet, suppository, and dispersible granule.

46. The composition according to any one of claims 42-44, comprising at least 5% to 70% of the compound.

47. The composition according to any one of claims 42-44 or 46, wherein the composition is formulated as a solution, suspension, emulsion, microsphere, or liposome.

48. The composition according to any one of claims 42-47, wherein the composition has a dosage of the compound from 0.1 mg to about 10,000 mg.

49. The composition according to any one of claims 42-47, wherein the composition has a dosage of the compound from about 25 mg to about 500 mg.

50. The composition according to any one of claims 42-49, wherein the excipient is selected from magnesium carbonate, magnesium stearate, talc, pectin, dextrin, starch, tragacanth, cocoa butter, lactose, sucrose, mannitol, sorbitol, starch, methyl cellulose, hydroxypropylmethylcellulose, sodium carboxymethylcellulose, gelatin, collagen, and any combination thereof.

51. A method comprising administering a compound according to any one of claims 1-41, or a composition according to any one of claims 42-50, to a subject.

52. The method according to claim 51, comprising:
administering a first dose of the compound, or a first dose of the composition, to a subject; and
administering at least a second dose of the compound, or at least a second dose of the composition.

53. The method according to claim 51 or 52, wherein the composition is formulated for administration to the subject 1 to 3 times per day.

54. The method according to any one of claims 51-53, wherein the composition is formulated for administration to the subject every 2 to 7 days.

55. The method according to any one of claims 51-54, wherein the compound, or the composition, is co-administered with a second active agent in a suitable weight ratio relative to the weight of the compound, or the composition, from about 1:100 to about 100:1 (w/w).

56. The method according to any one of claims 51-55, wherein the compound, or the composition, is administered to increase neuronal plasticity, to treat a neurological disease or to treat a neuropsychiatric disease.

57. The method according to claim 56, wherein the neurological disease is a migraine, a headache, a cluster headache, post-traumatic stress disorder (PTSD), anxiety, depression, neurodegenerative disorder, Alzheimer's disease, Parkinson's disease, a psychological disorder, treatment resistant depression, suicidal ideation, a major depressive disorder, bipolar disorder, schizophrenia, stroke, traumatic brain injury, or addiction.

58. The method according to claim 56, wherein the neuropsychiatric disease is a psychological disorder, treatment resistant depression, suicidal ideation, major depressive disorder, bipolar disorder, schizophrenia, post-traumatic stress disorder (PTSD), addiction, depression, or anxiety.

59. The method according to claim 51, wherein the compound, or the composition, is administered to treat fibromyalgia, muscle pain, joint stiffness, osteoarthritis, rheumatoid arthritis, or muscle cramps.

60. The method according to claim 51, wherein the compound, or the composition, is administered to treat premenstrual dysphoric disorder (PMDD), premenstrual syndrome (PMS), post-partum depression, or menopause.

61. The method according to claim 51, wherein the compound, or the composition, is administered to elicit a biological response by activating a 5-HT_{2A} receptor.

62. The method according to claim 61, comprising administering a 5-HT_{2A} antagonist in combination with at least one compound according to any one of claims 1-40 to mitigate undesirable effects of 5-HT_{2A} agonism.

63. The method according to claim 51, comprising administering at least one compound according to any one of claims 1-41, or the composition according to any one of claims 42-50, together with a serotonin receptor modulator selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, MDL-100,907, pimavanserin, nelotanserin and lorcaserin.

64. The method according to claim 51, comprising administering at least one compound according to any one of claims 1-41, or the composition according to any one of claims 42-50, together with one or more additional therapeutic agents selected from lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

65. The method according to claim 51, comprising administering at least one compound according to any one of claims 1-41, or the composition according to any one of claims 42-50, together with an empathogenic agent selected from N-Allyl-3,4-methylenedioxy-amphetamine (MDAL), N-Butyl-3,4-methylenedioxyamphetamine (MDBU), N-Benzyl-3,4-methylenedioxyamphetamine (MDBZ), N-Cyclopropylmethyl-3,4-methylenedioxyamphetamine (MDCPM), N,N-Dimethyl-3,4-methylenedioxyamphetamine (MDDM), N-Ethyl-3,4-methylenedioxyamphetamine (MDE; MDEA), N-(2-Hydroxyethyl)-3,4-methylenedioxy amphetamine (MDHOET), N-Isopropyl-3,4-methylenedioxyamphetamine (MDIP), N-Methyl-3,4-ethylenedioxyamphetamine (MDMC), N-Methoxy-3,4-methylenedioxyamphetamine (MDMEO), N-(2-Methoxyethyl)-3,4-methylenedioxyamphetamine (MDMEOET), alpha,alpha,N-Trimethyl-3,4-methylenedioxyphenethylamine (MDMP; 3,4-Methylenedioxy-N-methylphentermine), N-Hydroxy-3,4-methylenedioxyamphetamine (MDOH), 3,4-Methylenedioxyphenethylamine (MDPEA), alpha,alpha-Dimethyl-3,4-methylenedioxyphenethylamine (MDPH; 3,4-methylenedioxyphentermine), N-Propargyl-3,4-methylenedioxyamphetamine (MDPL), Methylenedioxy-2-aminoindane (MDAI), N-methyl-1,3-benzodioxolylbutanamine (MBDB), 3,4-methylenedioxy-N-methyl-α-ethylphenylethylamine, 3,4-Methylenedioxyamphetamine MDA, Methylone (also known as “3,4-methylenedioxy-N-methylcathinone), Ethylone, also known as 3,4-methylenedioxy-N-ethylcathinone, GHB (Gamma Hydroxybutyrate), sodium oxybate, N-Propyl-3,4-methylenedioxyamphetamine (MDPR), or a combination thereof.

66. The method according to claim 51, comprising administering at least one compound according to any one of claims 1-41, or the composition according to any one of claims 42-50, to increase at least one of translation, transcription, or secretion of neurotrophic factors.

67. A method for making a compound according to claim 1, comprising:
reacting an indole compound having a formula 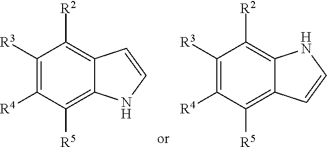
with an acylating agent to form a first intermediate comprising an acylated indole;
reacting the acylated indole with phthalimide, or a derivative thereof, followed by reaction with an amine reagent, or a protected amine, to form a second intermediate; and
converting the second intermediate into a compound according to any one of claims 1-40.

68. The method according to claim 67, wherein:
the amine is an amino alcohol having a formula 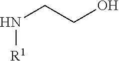
where R¹ is H, C₁₋₆ alkyl, or C₄₋₁₄ alkyl-cycloalkyl; and
converting comprises treating the second intermediate with a hydride reducing agent.

69. The method according to claim 67, wherein:
the amine is an amino sulfide having a formula 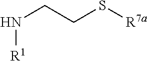
where R¹ and R^{7a} independently are H, C₁₋₆ alkyl, or C₄₋₁₄ alkyl-cycloalkyl; and
converting comprises treating the second intermediate with a hydride reducing agent to form an amino sulfide.

70. The method according to claim 69, further comprising:
oxidizing the amino sulfide with a peracid to form an oxidized intermediate; and
reducing the oxidized intermediate with a hydride reducing agent to form a sulfone.

71. The method according to claim 67, wherein:
the amine is an amino sulfide having a formula 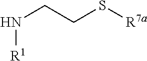
where R¹ and R^{7a} independently are H, C₁₋₆ alkyl, or C₄₋₁₄ alkyl-cycloalkyl; and
converting comprises first treating the second intermediate with an oxidizing agent to form an oxidized intermediate, followed by treating the oxidized intermediate with a hydride reducing agent to form the amino sulfoxide.

72. The method according to claim 67, wherein the amine is a diamine having a formula 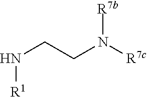
where R¹, R^{7a}, and R^{7b} independently are H, C₁₋₆ alkyl, C₄₋₁₄ alkyl-cycloalkyl or an amine protecting group.

73. The method according to claim 72, wherein at least one of R^{7a}, and R^{7b} is a t-butyloxycarbonyl protecting group.

74. The method according to claim 73, where converting comprises:
removing the amine protecting group to form a deprotected intermediate; and
reducing the deprotected intermediate with an a hydride reducing agent to form a diamine.

75. The method according to claim 74, further comprising acylating the diamine.
